(19) United States
(12) Patent Application Publication
(10) Pub. No.: US 2012/0084882 A1
(43) Pub. Date: Apr. 5, 2012

(54) NEMATODE-RESISTANT TRANSGENIC PLANTS

(75) Inventors: Aaron Wiig, Durham, NC (US); Bonnie McCaig, Durham, NC (US)

(73) Assignee: BASF Plant Science Company GmbH, Ludwigshafen (DE)

(21) Appl. No.: 13/138,695

(22) PCT Filed: Mar. 19, 2010
(86) PCT No.: PCT/EP2010/053606

§ 371 Date: Sep. 19, 2011

Related U.S. Application Data
(60) Provisional application No. 61/161,776, filed on Mar. 20, 2009.

(51) Int. Cl. C12N 15/82; A01H 5/00; C12N 15/63

(52) U.S. Cl. 800/279; 435/320.1; 800/301

(57) ABSTRACT

The present invention provides expression vectors encoding double stranded RNAs that target certain plant genes required for maintenance of parasitic nematode infection, nematode-resistant transgenic plants that express such double-stranded RNAs, and methods associated therewith. The targeted plant gene is a GLABRA-like gene, a homeodomain-like gene, a trehalose-6-phosphate phosphatase-like gene, an unknown gene having at least 80% homology to SEQ ID NO:16, a ringH2 finger-like gene, a zinc finger-like gene, or a MIOX-like gene.

This application claims priority benefit of U.S. provisional patent application Ser. No. 61/161,776, filed Mar. 20, 2009, the entire contents of which are incorporated herein by reference.

### BACKGROUND OF THE INVENTION

Nematodes are microscopic roundworms that feed on the roots, leaves and stems of more than 2,000 row crops, vegetables, fruits, and ornamental plants, causing an estimated $100 billion crop loss worldwide. A variety of parasitic nematode species infect crop plants, including root-knot nematodes (RKN), cyst- and lesion-forming nematodes. Root-knot nematodes, which are characterized by causing root gall formation at feeding sites, have a relatively broad host range and are therefore pathogenic on a large number of crop species. The cyst- and lesion-forming nematode species have a more limited host range, but still cause considerable losses in susceptible crops.

Pathogenic nematodes are present throughout the United States, with the greatest concentrations occurring in the warm, humid regions of the South and West and in sandy soils. Soybean cyst nematode (Heterodera glycines), the most serious pest of soybean plants, was first discovered in the United States in North Carolina in 1954. Some areas are so heavily infested by soybean cyst nematode (SCN) that soybean production is no longer economically possible without control measures. Although soybean is the major economic crop attacked by SCN, SCN parasitizes some fifty hosts in total, including field crops, vegetables, ornamentals, and weeds.

Signs of nematode damage include stunting and yellowing of leaves, and wilting of the plants during hot periods. However, nematode infestation can cause significant yield losses without any obvious above-ground disease symptoms. The primary causes of yield reduction are due to root damage underground. Roots infected by SCN are dwarfed or stunted. Nematode infestation also can decrease the number of nitrogen-fixing nodules on the roots, and may make the roots more susceptible to attacks by other soil-borne plant pathogens.

The nematode life cycle has three major stages: egg, juvenile, and adult. The life cycle varies between species of nematodes. For example, the SCN life cycle can usually be completed in 24 to 30 days under optimum conditions whereas other species can take as long as a year, or longer, to complete the life cycle. When temperature and moisture levels become favorable in the spring, worm-shaped juveniles hatch from eggs in the soil. Only nematodes in the juvenile developmental stage are capable of infecting soybean roots.

The life cycle of SCN has been the subject of many studies, and as such are a useful example for understanding the nematode life cycle. After penetrating soybean roots, SCN juveniles move through the root until they contact vascular tissue, at which time they stop migrating and begin to feed. With a stylet, the nematode injects secretions that modify certain root cells and transform them into specialized feeding sites. The root cells are morphologically transformed into large multinucleate syncytia (or giant cells in the case of RKN), which are used as a source of nutrients for the nematodes. The actively feeding nematodes thus steal essential nutrients from the plant resulting in yield loss. As female nematodes feed, they swell and eventually become so large that their bodies break through the root tissue and are exposed on the surface of the root.

After a period of feeding, male SCN nematodes, which are not swollen as adults, migrate out of the root into the soil and fertilize the enlarged adult females. The males then die, while the females remain attached to the root system and continue to feed. The eggs in the swollen females begin developing, initially in a mass or egg sac outside the body, and then later within the nematode body cavity. Eventually the entire adult female body cavity is filled with eggs, and the nematode dies. It is the egg-filled body of the dead female that is referred to as the cyst. Cysts eventually dislodge and are found free in the soil. The walls of the cyst become very tough, providing excellent protection for the approximately 200 to 400 eggs contained within. SCN eggs survive within the cyst until proper hatching conditions occur. Although many of the eggs may hatch within the first year, many also will survive within the protective cysts for several years.

A nematode can move through the soil only a few inches per year on its own power. However, nematode infestation can be spread substantial distances in a variety of ways. Anything that can move infested soil is capable of spreading the infestation, including farm machinery, vehicles and tools, wind, water, animals, and farm workers. Seed sized particles of soil often contaminate harvested seed. Consequently, nematode infestation can be spread when contaminated seed from infested fields is planted in non-infested fields. There is even evidence that certain nematode species can be spread by birds. Only some of these causes can be prevented.

Traditional practices for managing nematode infestation include: maintaining proper soil nutrients and soil pH levels in nematode-infested land; controlling other plant diseases, as well as insect and weed pests; using sanitation practices such as plowing, planting, and cultivating of nematode-infested fields only after working non-infested fields; cleaning equipment thoroughly with high pressure water or steam after working in infested fields; not using seed grown on infested land for planting non-infested fields unless the seed has been properly cleaned; rotating infested fields and alternating host crops with non-host crops; using nematicides; and planting resistant plant varieties. Methods have been proposed for the genetic transformation of plants in order to confer increased resistance to plant parasitic nematodes. U.S. Pat. Nos. 5,589,622 and 5,824,876 are directed to the identification of plant genes expressed specifically in or adjacent to the feeding site of the plant after attachment by the nematode. The promoters of these plant target genes can then be used to direct the specific expression of detrimental proteins or enzymes, or the expression of antisense RNA to the target gene or to general cellular genes. The plant promoters may also be used to confer nematode resistance specifically at the feeding site by transforming the plant with a construct comprising the promoter of the plant target gene linked to a gene whose product induces lethality in the nematode after ingestion.

Recently, RNA interference (RNAi), also referred to as gene silencing, has been proposed as a method for controlling nematodes. When double-stranded RNA (dsRNA) corresponding essentially to the sequence of a target gene or mRNA is introduced into a cell, expression from the target gene is inhibited (See e.g., U.S. Pat. No. 6,506,559). U.S. Pat. No. 6,506,559 demonstrates the effectiveness of RNAi against known genes in Caenorhabditis elegans, but does not demonstrate the usefulness of RNAi for controlling plant parasitic nematodes.

Use of RNAi to target essential nematode genes has been proposed, for example, in PCT Publication WO 01/96584, WO 01/17654, US 2004/0098761, US 2005/0091713, US 2005/0188438, US 2006/0037101, US 2006/0080749, US 2007/0199100, and US 2007/0250947. A number of models have been proposed for the action of RNAi. In mammalian systems, dsRNAs larger than 30 nucleotides trigger induction of interferon synthesis and a global shut-down of protein syntheses, in a non-sequence-specific manner. However, U.S. Pat. No. 6,506,559 discloses that in nematodes, the length of the dsRNA corresponding to the target gene sequence may be at least 25, 50, 100, 200, 300, or 400 bases, and that even larger dsRNAs were also effective at inducing RNAi in C. elegans. It is known that when hairpin RNA constructs comprising double stranded regions ranging from 98 to 854 nucleotides were transformed into a number of plant species, the target plant genes were efficiently silenced. There is general agreement that in many organisms, including nematodes and plants, large pieces of dsRNA are cleaved into about 19-24 nucleotide fragments (siRNA) within cells, and that these siRNAs are the actual mediators of the RNAi phenomenon.

Although there have been numerous efforts to use RNAi to control plant parasitic nematodes, to date no transgenic nematode-resistant plant has been deregulated in any country. Accordingly, there continues to be a need to identify safe and effective compositions and methods for the controlling plant parasitic nematodes using RNAi, and for the production of plants having increased resistance to plant parasitic nematodes.

### SUMMARY OF THE INVENTION

The present invention provides nucleic acids, transgenic plants, and methods to overcome or alleviate nematode infestation of valuable agricultural crops such as soybeans. The nucleic acids of the invention are capable of decreasing expression of plant target genes by RNA interference (RNAi). In accordance with the invention, the plant target gene is selected from a group consisting of a GLABRA-like gene, a homeodomain-like gene (HD-like), a trehalose-6-phosphate phosphatase-like gene (TPP-like), an unknown gene (UNK), a RingH2 finger-like gene (RingH2-like), a zinc finger-like gene (ZF-like), and a MIOX-like gene.

In one embodiment, the invention provides an isolated expression vector encoding a double stranded RNA comprising a first strand and a second strand complementary to the first strand, wherein the first strand is substantially identical to a portion of a plant target gene, the portion being selected from the group consisting of from about 19 to about 400 or 500 consecutive nucleotides of the target gene, wherein the double stranded RNA inhibits expression of the target gene, and wherein the target gene is selected from the group consisting of (a) a polynucleotide encoding a plant GLABRA-like protein having at least 80% sequence identity to a soybean GLABRA-like protein having a sequence as set forth in SEQ ID NO:2; (b) a polynucleotide encoding a plant homeodomain-like protein having at least 80% sequence identity to a soybean homeodomain-like protein having a sequence as set forth in SEQ ID NO:5 or SEQ ID NO:8; (c) a polynucleotide encoding a plant trehalose-6-phosphate phosphatase-like protein; (d) a polynucleotide encoding a plant unknown protein having at least 80% sequence identity to a soybean unknown protein having a sequence as set forth in SEQ ID NO:17; (e) a polynucleotide encoding a RingH2 finger-like protein having at least 80% sequence identity to a soybean RingH2 finger-like protein having a sequence as set forth in SEQ ID NO:20; (f) a polynucleotide encoding a zinc finger-like protein having at least 80% sequence identity to a soybean zinc finger-like protein having a sequence as set forth in SEQ ID NO:23 or SEQ ID NO:26; (g) a polynucleotide encoding a MIOX-like protein.

The invention is further embodied as an isolated expression vector comprising a nucleic acid encoding a pool of double stranded RNA molecules comprising a multiplicity of RNA molecules each comprising a double stranded region having a length of about 19, 20, 21, 22, 23, or 24 nucleotides, wherein said RNA molecules are derived from a polynucleotide selected from the group consisting of (a) a polynucleotide encoding a plant GLABRA-like protein having at least 80% sequence identity to a soybean GLABRA-like protein having a sequence as set forth in SEQ ID NO:2; (b) a polynucleotide encoding a plant homeodomain-like protein having at least 80% sequence identity to a soybean homeodomain-like protein having a sequence as set forth in SEQ ID NO:5 or SEQ ID NO:8; (c) a polynucleotide encoding a plant trehalose-6-phosphate phosphatase-like protein; (d) a polynucleotide encoding a plant unknown protein having at least 80% sequence identity to a soybean unknown protein having a sequence as set forth in SEQ ID NO:17; (e) a polynucleotide encoding a RingH2 finger-like protein having at least 80% sequence identity to a soybean RingH2 finger-like protein having a sequence as set forth in SEQ ID NO:20; (f) a polynucleotide encoding a zinc finger-like protein having at least 80% sequence identity to a soybean zinc finger-like protein having a sequence as set forth in SEQ ID NO:23 or SEQ ID NO:26; (g) a polynucleotide encoding a MIOX-like protein.

In another embodiment, the invention provides a transgenic plant capable of expressing at least one a dsRNA that is substantially identical to a portion of a plant target gene selected from the group consisting of (a) a polynucleotide encoding a plant GLABRA-like protein having at least 80% sequence identity to a soybean GLABRA-like protein having a sequence as set forth in SEQ ID NO:2; (b) a polynucleotide encoding a plant homeodomain-like protein having at least 80% sequence identity to a soybean homeodomain-like protein having a sequence as set forth in SEQ ID NO:5 or SEQ ID NO:8; (c) a polynucleotide encoding a plant trehalose-6-phosphate phosphatase-like protein; (d) a polynucleotide encoding a plant unknown protein having at least 80% sequence identity to a soybean unknown protein having a sequence as set forth in SEQ ID NO:17; (e) a polynucleotide encoding a RingH2 finger-like protein having at least 80% sequence identity to a soybean RingH2 finger-like protein having a sequence as set forth in SEQ ID NO:20; (f) a polynucleotide encoding a zinc finger-like protein having at least 80% sequence identity to a soybean zinc finger-like protein having a sequence as set forth in SEQ ID NO:23 or SEQ ID NO:26; (g) a polynucleotide encoding a MIOX-like protein, wherein the dsRNA inhibits expression of the target gene in the plant root.

The invention further encompasses a method of making a transgenic plant capable of expressing a dsRNA comprising a first strand that is substantially identical to portion of a plant target gene and a second strand complementary to the first strand, wherein the target gene is selected from the group consisting of (a) a polynucleotide encoding a plant GLABRA-like protein having at least 80% sequence identity to a soybean GLABRA-like protein having a sequence as set forth in SEQ ID NO:2; (b) a polynucleotide encoding a plant homeodomain-like protein having at least 80% sequence identity to a soybean homeodomain-like protein having a sequence as set forth in SEQ ID NO:5 or SEQ ID NO:8; (c) a polynucleotide encoding a plant trehalose-6-phosphate phosphatase-like protein; (d) a polynucleotide encoding a plant unknown protein having at least 80% sequence identity to a soybean unknown protein having a sequence as set forth in SEQ ID NO:17; (e) a polynucleotide encoding a RingH2 finger-like protein having at least 80% sequence identity to a soybean RingH2 finger-like protein having a sequence as set forth in SEQ ID NO:20; (f) a polynucleotide encoding a zinc finger-like protein having at least 80% sequence identity to a soybean zinc finger-like protein having a sequence as set forth in SEQ ID NO:23 or SEQ ID NO:26; (g) a polynucleotide encoding a MIOX-like protein, said method comprising the steps of: (h) preparing an expression vector comprising a nucleic acid encoding the dsRNA, wherein the nucleic acid is able to form a double-stranded transcript once expressed in the plant; (ii) transforming a recipient plant with said expression vector; (iii) producing one or more transgenic offspring of said recipient plant; and (iv) selecting the offspring for resistance to nematode infection.

The invention further provides a method of conferring nematode resistance to a plant, said method comprising the steps of: ( ) selecting a plant target gene selected from the group consisting of (a) a polynucleotide encoding a plant GLABRA-like protein having at least 80% sequence identity to a soybean GLABRA-like protein having a sequence as set forth in SEQ ID NO:2; (b) a polynucleotide encoding a plant homeodomain-like protein having at least 80% sequence identity to a soybean homeodomain-like protein having a sequence as set forth in SEQ ID NO:5 or SEQ ID NO:8; (c) a polynucleotide encoding a plant trehalose-6-phosphate phosphatase-like protein; (d) a polynucleotide encoding a plant unknown protein having at least 80% sequence identity to a soybean unknown protein having a sequence as set forth in SEQ ID NO:17; (e) a polynucleotide encoding a RingH2 finger-like protein having at least 80% sequence identity to a soybean RingH2 finger-like protein having a sequence as set forth in SEQ ID NO:20; (f) a polynucleotide encoding a zinc finger-like protein having at least 80% sequence identity to a soybean zinc finger-like protein having a sequence as set forth in SEQ ID NO:23 or SEQ ID NO:26; (g) a polynucleotide encoding a MIOX-like protein; (ii) preparing an expression vector comprising a nucleic acid encoding a dsRNA comprising a first strand that is substantially identical to a portion of the target gene and a second strand complementary to the first strand, wherein the nucleic acid is able to form a double-stranded transcript once expressed in the plant; (iii) transforming a recipient plant with said nucleic acid; (iv) producing one or more transgenic offspring of said recipient plant; and (v) selecting the offspring for nematode resistance.

### BRIEF DESCRIPTION OF THE DRAWINGS

FIG. 1 shows the table of SEQ ID NOs assigned to corresponding nucleotide and amino acid sequences from Glycine max and other plant species.

FIG. 2 shows the amino acid alignment of the open reading frame encoded by GmHD-like (SEQ ID NO:5) with a related soybean amino acid sequence GM50634465 (SEQ ID NO:8), using the Vector NTI software suite v10.3.0 (gap opening penalty=10, gap extension penalty=0.05, gap separation penalty=8). The hairpin stem generated by RAW484 with the sense strand described by SEQ ID NO:6 can target the corresponding DNA sequences described by SEQ ID NO:4 and SEQ ID NO:7.

FIG. 3 shows the amino acid alignment of the open reading frame encoded by GmTPP-like (SEQ ID NO:10) with related soybean amino acid sequences GM47125400 (SEQ ID NO:13) and GMsq97c08 (SEQ ID NO:15), using the Vector NTI software suite v10.3.0 (gap opening penalty=10, gap extension penalty=0.05, gap separation penalty=8). The hairpin stem generated by RTJ150 with the sense strand described by SEQ ID NO:11 can target the corresponding DNA sequences described by SEQ ID NO:9, SEQ ID NO:12, and SEQ ID NO:14.

FIG. 4 shows the amino acid alignment of the open reading frame encoded by GmZF-like (SEQ ID NO:23) with a related soybean amino acid sequence described by soybean gene index identifier TC248286 (SEQ ID NO:26), using the Vector NTI software suite v10.3.0 (gap opening penalty=10, gap extension penalty=0.05, gap separation penalty=8). The hairpin stem generated by RAW486 with the sense strand described by SEQ ID NO:24 can target the corresponding DNA sequences described by SEQ ID NO:22 and SEQ ID NO:25.

FIG. 5 shows the amino acid alignment of the open reading frame encoded by GmMIOX-like (SEQ ID NO:28) with a related soybean amino acid sequence GM50229820 (SEQ ID NO:31), using the Vector NTI software suite v10.3.0 (gap opening penalty=10, gap extension penalty=0.05, gap separation penalty=8). The hairpin stem generated by RTP2615-1 with the sense strand described by SEQ ID NO:29 can target the corresponding DNA sequences described by SEQ ID NO:27 and SEQ ID NO:30.

FIG. 6a-c shows the DNA alignment of GmHD-like (SEQ ID NO:4) with a related soybean sequence GM50634465 (SEQ ID NO:7), using the Vector NTI software suite v10.3.0 (gap opening penalty=15, gap extension penalty=6.66, gap separation penalty=8). The hairpin stem generated by RAW484 with the sense strand described by SEQ ID NO:6 can target the corresponding DNA sequences described by SEQ ID NO:4 and SEQ ID NO:7 as shown in the alignment

FIG. 7a-e shows the DNA alignment of GmTPP-like (SEQ ID NO:9) with related DNA sequences GM47125400 (SEQ ID NO:12) and GMsq97c08 (SEQ ID NO:14), using the Vector NTI software suite v10.3.0 (gap opening penalty=15, gap extension penalty=6.66, gap separation penalty=8). The hairpin stem generated by RTJ150 with the sense strand described by SEQ ID NO:11 can target the corresponding DNA sequences described by SEQ ID NO:9, SEQ ID NO:12, and SEQ ID NO:14 as shown in the alignment.

FIG. 8a-c shows the DNA alignment of GmZF-like (SEQ ID NO:22) with a related soybean DNA sequence described by soybean gene index identifier TC248286 (SEQ ID NO:25), using the Vector NTI software suite v10.3.0 (gap opening penalty=15, gap extension penalty=6.66, gap separation penalty=8). The hairpin stem generated by RAW486 with the sense strand described by SEQ ID NO:24 can target the corresponding DNA sequences described by SEQ ID NO:22 and SEQ ID NO:25 as shown in the alignment.

FIG. 9a-c shows the DNA alignment of GmMIOX-like SEQ ID NO:27 with a related soybean DNA sequence GM50229820 (SEQ ID NO:30), using the Vector NTI software suite v10.3.0 (gap opening penalty=15, gap extension penalty=6.66, gap separation penalty=8). The hairpin stem generated by RTP2615-1 with the sense strand described by SEQ ID NO:29 can target the corresponding DNA sequences described by SEQ ID NO:27 and SEQ ID NO:30 as shown in the alignment.

FIGS. 10a-h show global percent identity of exemplary GmHD-like sequences (FIG. 10a, amino acid; FIG. 10b, nucleotide), GmTPP-like sequences (FIG. 10c, amino acid; FIG. 10d, nucleotide), GmZF-like sequences (FIG. 10e, amino acid; FIG. 10f, nucleotide), and GmMIOX-like sequences (FIG. 10g, amino acid; FIG. 10h, nucleotide). Percent identity was calculated from multiple alignments using the Vector NTI software suite v10.3.0.

FIG. 11 shows the amino acid alignment of the GmMIOX-like gene (SEQ ID NO:28) with related homologs from cotton TC86807 and TC86837 (SEQ ID NO:33 and SEQ ID NO:35, respectively), sugar beet TC6112 (SEQ ID NO:37), corn ZM2G126900 (SEQ ID NO:39), and potato gene index identifier CV505571 (SEQ ID NO:41) using the Vector NTI software suite v10.3.0 (gap opening penalty=15, gap extension penalty=6.66, gap separation penalty=8).

FIG. 12 shows the nucleotide alignment of the GmMIOX-like gene (SEQ ID NO:27) with related homologs from cotton TC86807 and TC86837 (SEQ ID NO:32 and SEQ ID NO:34, respectively), sugar beet TC6112 (SEQ ID NO:36), corn ZM2G126900 (SEQ ID NO:38), and potato gene index identifier CV505571 (SEQ ID NO:40) using the Vector NTI software suite v10.3.0 (gap opening penalty=15, gap extension penalty=6.66, gap separation penalty=8).

FIGS. 13a-b show global percent identity of exemplary MIOX-like sequences (FIG. 13a, amino acid; FIG. 13b, nucleotide). Percent identity was calculated from multiple alignments using the Vector NTI software suite v10.3.0.

FIGS. 14a-14t show various 21 mers possible in SEQ ID NO:1, 3, 4, 6, 7, 9, 11, 12, 14, 16, 18, 19, 21, 22, 24, 25, 27, 29, 30, 32, 34, 36, 38, or 40 by nucleotide position.

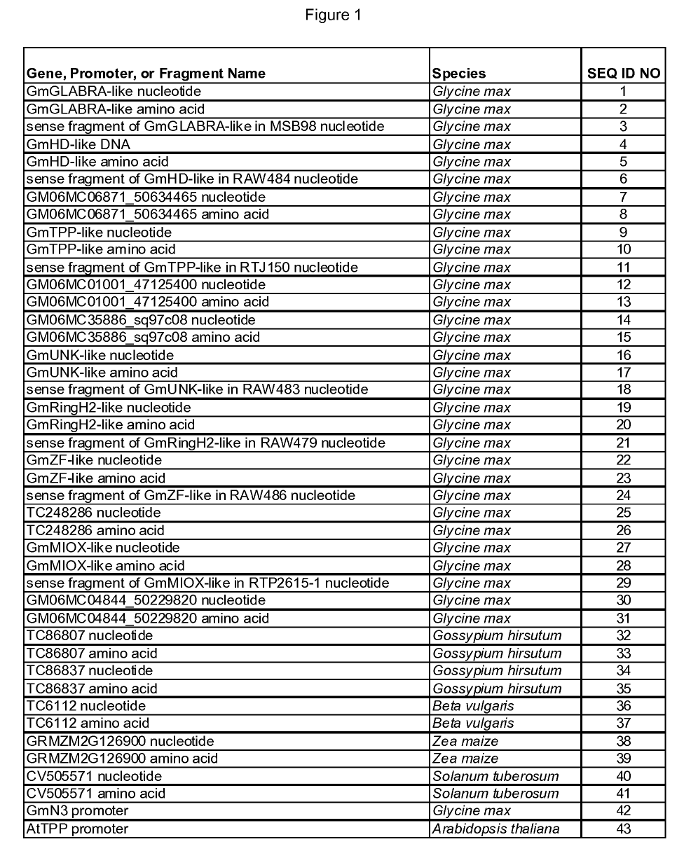

### DETAILED DESCRIPTION OF THE PREFERRED EMBODIMENTS

The present invention may be understood more readily by reference to the following detailed description of the preferred embodiments of the invention and the examples included herein. Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5th Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “a cell” can mean that at least one cell can be utilized It is to be understood that the terminology used herein is for the purpose of describing specific embodiments only and is not intended to be limiting. Throughout this application, various publications are referenced. The disclosures of all of these publications and those references cited within those publications in their entireties are hereby incorporated by reference into this application in order to more fully describe the state of the art to which this invention pertains. Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth Enzymol. 68; Wu et al., (Eds.) 1983 Meth. Enzymol. 100 and 101; Grossman and Moldave (Eds.) 1980 Meth. Enzymol. 65; Miller (Ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

As used herein, the term “expression vector” refers to a nucleic acid molecule capable of (i) transporting another nucleic acid to which it has been linked and (ii) directing the expression of polynucleotides to which they are operatively linked. As used herein, the terms “operatively linked” and “in operative association” are interchangeable and are intended to mean that the nucleotide sequence of interest is linked to regulatory sequence(s) of the expression vector in a manner which allows expression of the nucleotide sequence in a host cell when the vector is introduced into the host cell. The term “regulatory sequence” is intended to include promoters, enhancers, and other expression control elements (e.g., polyadenylation signals).

As used herein, “RNAi” or “RNA interference” refers to the process of sequence-specific post-transcriptional gene silencing in plants, mediated by double-stranded RNA (dsRNA). As used herein, “dsRNA” refers to RNA that is partially or completely double stranded. Double stranded RNA is also referred to as short interfering RNA (sRNA), short interfering nucleic acid (siNA), micro-RNA (miRNA), and the like. In the RNAi process, dsRNA comprising a first strand that is substantially identical to a portion of a target gene and a second strand that is complementary to the first strand is introduced into a plant. After introduction into the plant, the target gene-specific dsRNA is processed into relatively small fragments (siRNAs) by a plant cell containing the RNAi processing machinery resulting in target gene silencing.

As used herein, taking into consideration the substitution of uracil for thymine when comparing RNA and DNA sequences, the term “substantially identical” as applied to dsRNA means that the nucleotide sequence of one strand of the dsRNA is at least about 80%-90% identical to 20 or more contiguous nucleotides of the target gene, more preferably, at least about 90-95% identical to 20 or more contiguous nucleotides of the target gene, and most preferably at least about 95%, 96%, 97%, 98% or 99% identical or absolutely identical to 20 or more contiguous nucleotides of the target gene. 20 or more nucleotides means a portion, being at least about 20, 21, 22, 23, 24, 25, 50, 100, 200, 300, 400, 500, 1000, 1500, consecutive bases or up to the full length of the target gene.

As used herein, “complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other. As used herein, the term “substantially complementary” means that two nucleic acid sequences are complementary over at least at 80% of their nucleotides. Preferably, the two nucleic acid sequences are complementary over at least at 85%, 90%, 95%, 96%, 97%, 98%, 99% or more or all of their nucleotides. Alternatively, “substantially complementary” means that two nucleic acid sequences can hybridize under high stringency conditions. As used herein, the term “substantially identical” or “corresponding to” means that two nucleic acid sequences have at least 80% sequence identity. Preferably, the two nucleic acid sequences have at least 85%, 90%, 95%, 96%, 97%, 98%, 99% or 100% of sequence identity.

Also as used herein, the terms “nucleic acid” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. An “isolated” nucleic acid molecule is one that is substantially separated from other nucleic acid molecules which are present in the natural source of the nucleic acid (i.e., sequences encoding other polypeptides). For example, a cloned nucleic acid is considered isolated. A nucleic acid is also considered isolated if it has been altered by human intervention, or placed in a locus or location that is not its natural site, or if it is introduced into a cell by transformation. Moreover, an isolated nucleic acid molecule, such as a cDNA molecule, can be free from some of the other cellular material with which it is naturally associated, or culture medium when produced by recombinant techniques, or chemical precursors or other chemicals when chemically synthesized. While it may optionally encompass untranslated sequence located at both the 3′ and 5′ ends of the coding region of a gene, it may be preferable to remove the sequences which naturally flank the coding region in its naturally occurring replicon.

As used herein, the terms “contacting” and “administering” are used interchangeably, and refer to a process by which dsRNA of the present invention is transcribed in a plant in order to inhibit expression of an essential target gene in the plant. The dsRNA may be administered in a number of ways, including, but not limited to, direct introduction into a cell (i.e., intracellularly); or extracellular introduction, or into the vascular system of the plant, or the dsRNA may be transcribed by the plant. For example, the dsRNA may be sprayed onto a plant, or the dsRNA may be applied to soil in the vicinity of roots, taken up by the plant, or a plant may be genetically engineered to express the dsRNA targeting a plant target gene in an amount sufficient to kill or adversely affect some or all of the parasitic nematode to which the plant is exposed by dsRNA silencing (RNAi) of the plant target gene.

As used herein, the term “control,” when used in the context of an infection, refers to the reduction or prevention of an infection. Reducing or preventing an infection by a nematode will cause a plant to have increased resistance to the nematode; however, such increased resistance does not imply that the plant necessarily has 100% resistance to infection. In preferred embodiments, the resistance to infection by a nematode in a resistant plant is greater than 10%, 20%, 30%, 40%, 50%, 60%, 70%, 80%, 90%, or 95% in comparison to a wild type plant that is not resistant to nematodes. Preferably the wild type plant is a plant of a similar, more preferably identical genotype as the plant having increased resistance to the nematode, but does not comprise a dsRNA directed to the target gene. The plant's resistance to infection by the nematode may be due to the death, sterility, arrest in development, or impaired mobility of the nematode upon exposure to the dsRNA specific to a plant gene having some effect on feeding site development, maintenance, or overall ability of the feeding site to provide nutrition to the nematode. The term “resistant to nematode infection” or “a plant having nematode resistance” as used herein refers to the ability of a plant, as compared to a wild type plant, to avoid infection by nematodes, to kill nematodes or to hamper, reduce or stop the development, growth or multiplication of nematodes. This might be achieved by an active process, e.g. by producing a substance detrimental to the nematode, or by a passive process, like having a reduced nutritional value for the nematode or not developing structures induced by the nematode feeding site like syncytia or giant cells. The level of nematode resistance of a plant can be determined in various ways, e.g. by counting the nematodes being able to establish parasitism on that plant, or measuring development times of nematodes, proportion of male and female nematodes or, for cyst nematodes, counting the number of cysts or nematode eggs produced on roots of an infected plant or plant assay system.

The term “plant” is intended to encompass plants at any stage of maturity or development, as well as any tissues or organs (plant parts) taken or derived from any such plant unless otherwise clearly indicated by context. Plant parts include, but are not limited to, stems, roots, flowers, ovules, stamens, seeds, leaves, embryos, meristematic regions, callus tissue, anther cultures, gametophytes, sporophytes, pollen, microspores, protoplasts, hairy root cultures, and the like. The present invention also includes seeds produced by the plants of the present invention. In one embodiment, the seeds are true breeding for an increased resistance to nematode infection as compared to a wild-type variety of the plant seed. As used herein, a “plant cell” includes, but is not limited to, a protoplast, gamete producing cell, and a cell that regenerates into a whole plant. Tissue culture of various tissues of plants and regeneration of plants therefrom is well known in the art and is widely published.

As used herein, the term “transgenic” refers to any plant, plant cell, callus, plant tissue, or plant part that contains all or part of at least one recombinant polynucleotide. In many cases, all or part of the recombinant polynucleotide is stably integrated into a chromosome or stable extra-chromosomal element, so that it is passed on to successive generations. For the purposes of the invention, the term “recombinant polynucleotide” refers to a polynucleotide that has been altered, rearranged, or modified by genetic engineering. Examples include any cloned polynucleotide, or polynucleotides, that are linked or joined to heterologous sequences. The term “recombinant” does not refer to alterations of polynucleotides that result from naturally occurring events, such as spontaneous mutations, or from non-spontaneous mutagenesis followed by selective breeding.

As used herein, the term “amount sufficient to inhibit expression” refers to a concentration or amount of the dsRNA that is sufficient to reduce levels or stability of mRNA or protein produced from a target gene in a plant. As used herein, “inhibiting expression” refers to the absence or observable decrease in the level of protein and/or mRNA product from a target gene. Inhibition of the plant target gene expression may result in lethality to the parasitic nematode, or such inhibition may delay or prevent entry into a particular developmental step (e.g., metamorphosis), if plant disease is associated with a particular stage of the parasitic nematode's life cycle. The consequences of inhibition can be confirmed by examination of the outward properties of the nematode (as presented below in the examples).

The invention is embodied in an isolated expression vector encoding at least one dsRNA capable of specifically inhibiting expression of a plant target gene that effects nematode feeding site development, feeding site maintenance, nematode survival, nematode metamorphosis, or nematode reproduction. The dsRNA encoded by the expression vector of the invention comprises a first strand and a second strand complementary to the first strand, wherein the first strand is substantially identical to a portion of a plant target gene. The first strand of the dsRNA may be substantially identical to any portion of the target gene, so long as expression of the target gene in the plant is inhibited. Preferably, the first strand of the dsRNA is substantially identical to from about 19, 20, or 21 to about 400 or 500 consecutive nucleotides of the target gene.

The expression vector of the invention comprises a nucleic acid encoding the dsRNA operatively linked to a regulatory sequence which is a promoter. Any promoter may be employed in the isolated expression vector of the invention. Preferably, the nucleic acid encoding the dsRNA is under the transcriptional control of a root specific promoter or a parasitic nematode induced feeding cell-specific promoter. More preferably, the expression vector comprises a nucleic acid encoding the dsRNA in operative association with a parasitic nematode induced feeding cell-specific promoter.

In one embodiment, the isolated expression vector of the invention encodes a dsRNA capable of inhibiting expression of a plant GLABRA-like target gene. GLABRA genes are part of a family of HD-ZIP IV transcription factors. GLABRA transcription factors in plants have been shown to be involved with accumulation of anthocyanin, root development, and trichome development. In this embodiment the dsRNA encoded by the expression vector of the invention comprises a first strand that is substantially identical to a portion of the GLABRA-like target gene of a plant genome and a second strand that is substantially complementary to the first strand.

As shown in Example 1, the full length G. max GLABRA-like target gene was isolated and is represented in SEQ ID NO:1. In this embodiment, the plant GLABRA-like target gene is selected from the group consisting of: (a) a polynucleotide encoding a plant GLABRA-like protein having at least 80% sequence identity to a soybean GLABRA-like protein having a sequence as set forth in SEQ ID NO:2 (b) a polynucleotide having a sequence as set forth in SEQ ID NO:1, (c) a polynucleotide having at least 80% sequence identity to SEQ ID NO:1; (d) a polynucleotide from a plant that hybridizes under stringent conditions to the sequence set forth in SEQ ID NO:1. An exemplary dsRNA first strand that is substantially identical to a portion of the soybean GLABRA-like target gene, which is suitable for use in the expression vector of the invention, is set forth in SEQ ID NO:3.

In another embodiment, the isolated expression vector of the invention encodes a dsRNA capable of inhibiting expression of a plant homeodomain-like target gene. Homeodomain like genes contain a DNA binding domain and are generally considered to be transcription factors. In this embodiment, the dsRNA encoded by the expression vector of the invention comprises a first strand that is substantially identical to a portion of the homeodomain-like target gene of a plant genome and a second strand that is substantially complementary to the first strand. As shown in Example 1, the full length G. max homeodomain-like target gene was isolated and is represented in SEQ ID NO:4. In this embodiment, the plant homeodomain-like target gene is selected from the group consisting of (a) a polynucleotide encoding a plant homeodomain-like protein having at least 80% sequence identity to a soybean homeodomain-like protein having a sequence as set forth in SEQ ID NO:5 or SEQ ID NO:8; (b) a polynucleotide having a sequence as set forth in SEQ ID NO:4 or SEQ ID NO:7, (c) a polynucleotide having at least 80% sequence identity to SEQ ID NO:4 or SEQ ID NO:7; and (d) a polynucleotide from a plant that hybridizes under stringent conditions to the sequence set forth in SEQ ID NO:4 or SEQ ID NO:7. An exemplary dsRNA first strand that is substantially identical to a portion of the soybean homeodomain-like target gene, which is suitable for use in the expression vector of the invention, is set forth in SEQ ID NO:6.

In another embodiment, the isolated expression vector of the invention encodes a dsRNA capable of inhibiting expression of a plant trehalose-6-phosphate phosphatase-like (TPP) target gene. Plant TPP genes are involved with trehalose metabolism. In plants, trehalose has been shown to be an important sugar that is involved with stress response and physiology as an osmo-protectant and signaling molecule. The TPP enzyme converts trehalose-6-phostphate to trehalose. As shown in Example 1, the full length G. max trehalose-6-phosphate phosphatase-like gene was isolated and is represented in SEQ ID NO:9. In this embodiment, the dsRNA encoded by the expression vector of the invention comprises a first strand that is substantially identical to a portion of the trehalose-6-phosphate phosphatase-like target gene of a plant genome and a second strand that is substantially complementary to the first strand. The expression vector of this embodiment encodes a dsRNA capable of inhibiting any plant trehalose-6-phosphate phosphatase-like gene. Preferably, the dsRNA of this embodiment targets a soybean trehalose-6-phosphate phosphatase-like gene selected from the group consisting of: (a) a polynucleotide encoding a plant TPP-like protein having at least 80% sequence identity to a soybean TPP-like protein having a sequence as set forth in SEQ ID NO:10, SEQ ID NO:13, or SEQ ID NO:15; (b) a polynucleotide having a sequence as set forth in SEQ ID NO:9, SEQ ID NO:12, or SEQ ID NO:14, (c) a polynucleotide having at least 80% sequence identity to SEQ ID NO:9, SEQ ID NO:12, or SEQ ID NO:14 and (d) a polynucleotide from a plant that hybridizes under stringent conditions to the sequence set forth in SEQ ID NO:9, SEQ ID NO:12, or SEQ ID NO:14. An exemplary dsRNA first strand that is substantially identical to a portion of a soybean TPP-like target gene, which is suitable for use in the expression vector of the invention, is set forth in SEQ ID NO:11.

In another embodiment, the isolated expression vector of the invention encodes a dsRNA capable of inhibiting expression of a plant gene of unknown function which is a homolog of the soybean gene of unknown function having a full-length sequence as defined by SEQ ID NO:16. In this embodiment, the dsRNA encoded by the expression vector of the invention comprises a first strand that is substantially identical to a portion of the unknown target gene defined by SEQ ID NO:16, or a homolog thereof, and a second strand that is complementary to the first strand. In this embodiment, the dsRNA targets an unknown gene selected from the group consisting of: (a) a plant unknown protein having at least 80% sequence identity to a soybean unknown protein having a sequence as set forth in SEQ ID NO:17; (b) a polynucleotide having a sequence as set forth in SEQ ID NO:16, (c) a polynucleotide having at least 80% sequence identity to SEQ ID NO:16 and (d) a polynucleotide from a plant that hybridizes under stringent conditions to the sequence set forth in SEQ ID NO:16. An exemplary dsRNA first strand that is substantially identical to a portion of a soybean unknown target gene, which is suitable for use in the expression vector of the invention, is set forth in SEQ ID NO:18.

In another embodiment, the isolated expression vector of the invention encodes a dsRNA capable of inhibiting expression of a plant ringH2 finger-like target gene. Many plant RingH2 finger proteins are involved with a variety of plant processes including abiotic and biotic stress response, development, photorespiration, programmed cell death, seed germination, and cell cycle regulation. In this embodiment, the dsRNA encoded by the expression vector of the invention comprises a first strand that is substantially identical to a portion of the ringH2 finger-like target gene of a plant genome and a second strand that is complementary to the first strand. As shown in Example 1, the full length G. max ringH2 finger-like gene was isolated and is represented in SEQ ID NO:19. In this embodiment, the plant ringH2 finger-like target gene is selected from the group consisting of: (a) a polynucleotide encoding a RingH2 finger-like protein having at least 80% sequence identity to a soybean RingH2 finger-like protein having a sequence as set forth in SEQ ID NO:20; (b) a polynucleotide having a sequences as set forth in SEQ ID NO:19; (c) a polynucleotide having at least 80% sequence identity to SEQ ID NO:19; and (d) a polynucleotide from a plant that hybridizes under stringent conditions to the sequence set forth in SEQ ID NO:19. An exemplary dsRNA first strand that is substantially identical to a portion of a soybean RingH2 finger target gene, which is suitable for use in the expression vector of the invention, is set forth in SEQ ID NO:21.

In another embodiment, the isolated expression vector of the invention encodes a dsRNA capable of inhibiting expression of a plant zinc finger-like target gene. Zinc finger motif containing genes are involved with a variety of plant processes, including protein-protein interactions and DNA binding. In this embodiment, the dsRNA encoded by the expression vector of the invention comprises a first strand that is substantially identical to a portion of the zinc finger-like target gene of a plant genome and a second strand that is substantially complementary to the first strand. As shown in Example 1, the full length G. max zinc finger-like gene was isolated and is represented in SEQ ID NO:22. In this embodiment, the soybean zinc finger-like target gene is selected from the group consisting of: (a) a polynucleotide encoding a zinc finger-like protein having at least 80% sequence identity to a soybean zinc finger-like protein having a sequence as set forth in SEQ ID NO:23 or SEQ ID NO:26; (b) a polynucleotide having a sequence as set forth in SEQ ID NO:22 or SEQ ID NO:25, (c) a polynucleotide having at least 80% sequence identity to SEQ ID NO:22 or SEQ ID NO:25 and (d) a polynucleotide from a plant that hybridizes under stringent conditions to the sequence set forth in SEQ ID NO:22 or SEQ ID NO:25. An exemplary dsRNA first strand that is substantially identical to a portion of a soybean zinc finger-like target gene, which is suitable for use in the expression vector of the invention, is set forth in SEQ ID NO:24.

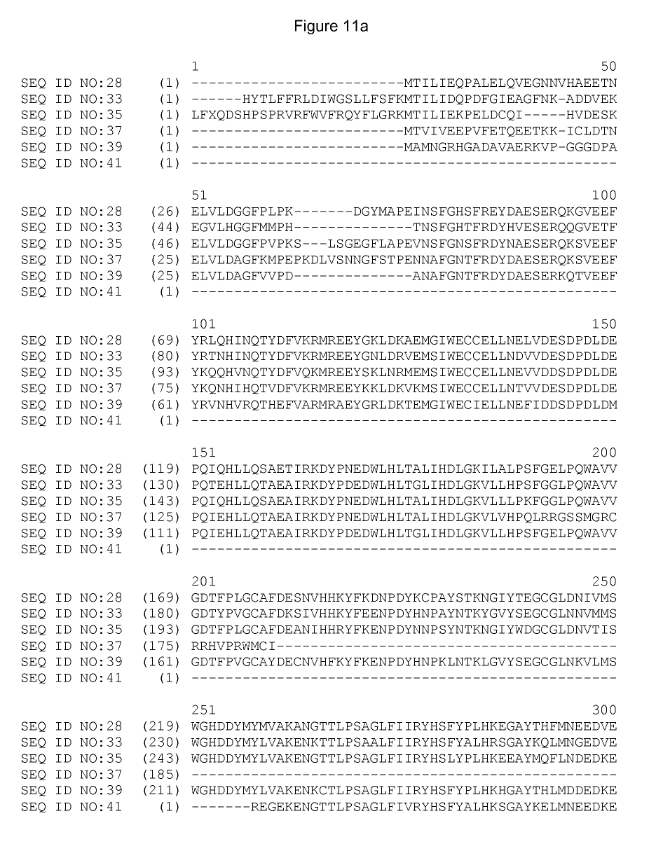

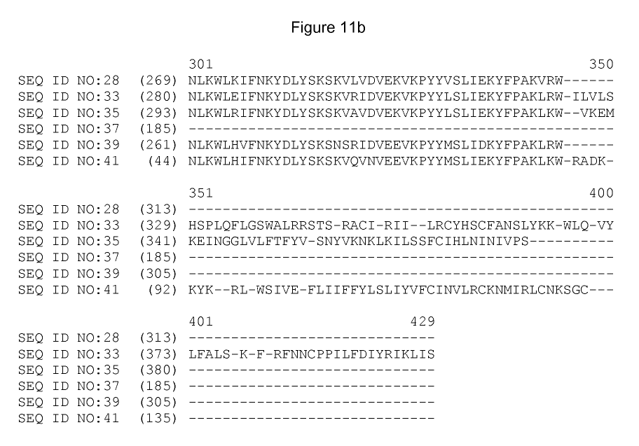

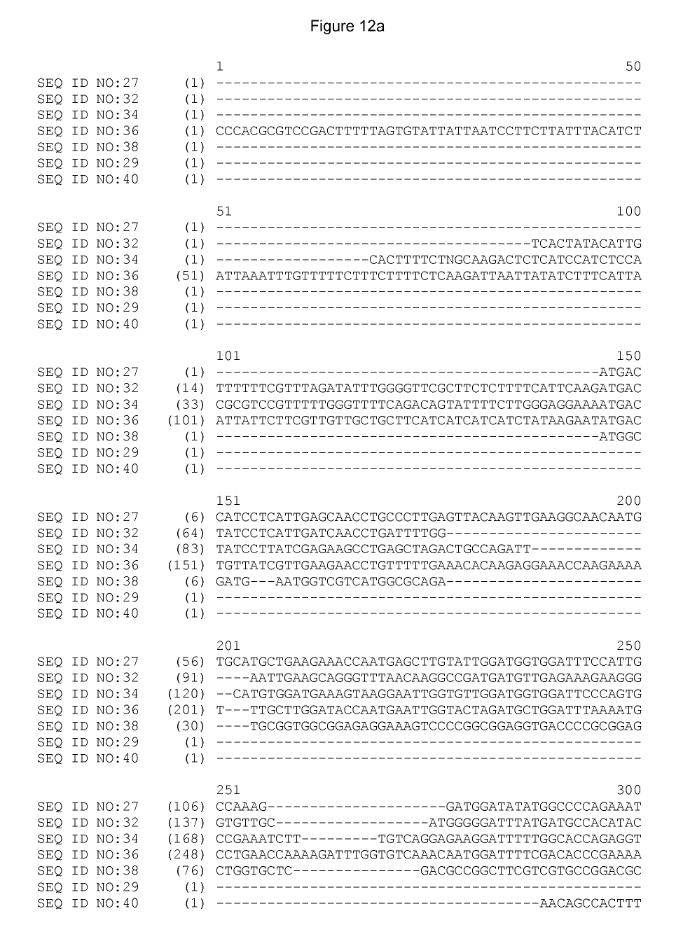

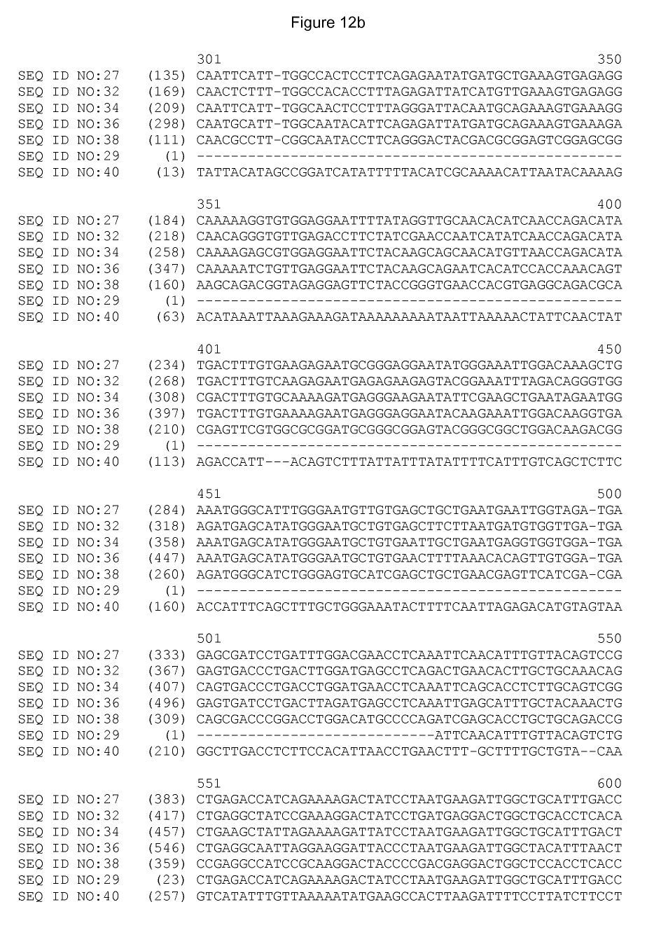

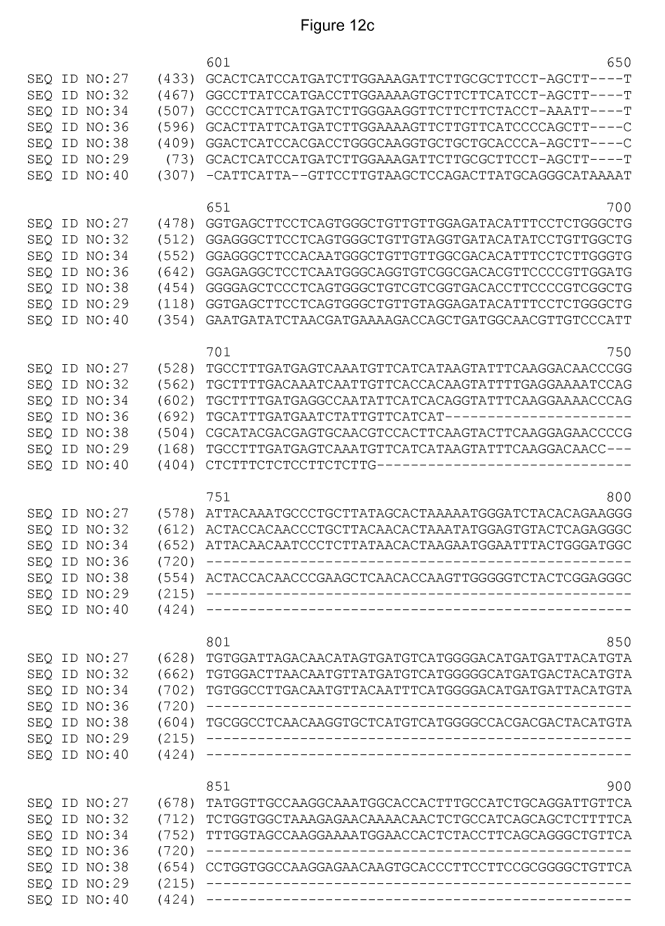

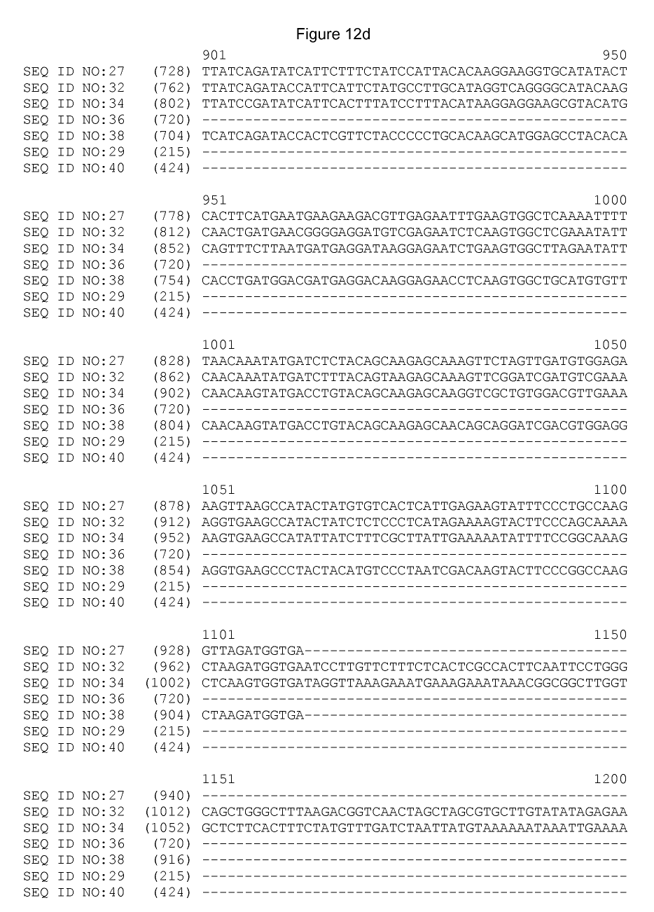

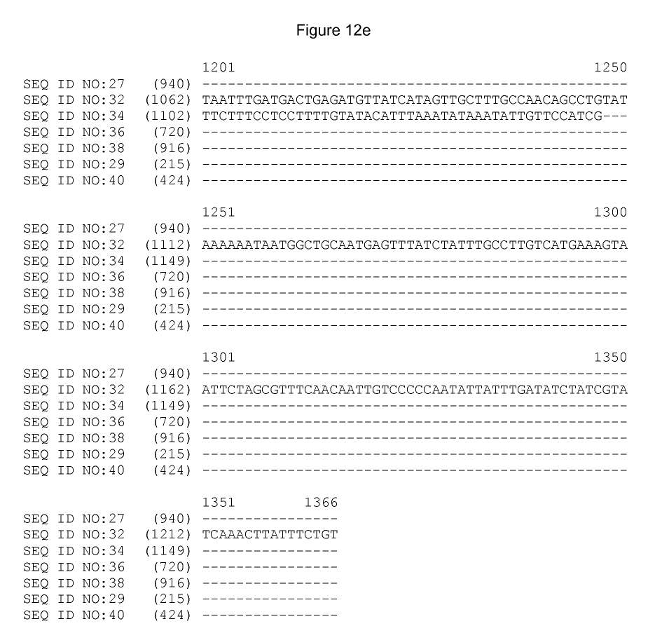

In another embodiment, the isolated expression vector of the invention encodes a dsRNA capable of inhibiting expression of a plant MIOX-like gene. Myo-inositol oxygenase (MIOX) is a key enzyme in cell wall polymer synthesis, regulating one of the two pathways involved in hemicellulose and pectin biosynthesis. MIOX catalyzes the cleavage of myo-inositol to glucuronic acid, which is then converted in a two-step process to Urdine-diphospho-glucuronic acid (UDP-GIcA). MIOX is highly conserved across plant and animal kingdoms, it is found as a single copy gene or a small gene family in all plants screened to date. In this embodiment, the dsRNA encoded by the expression vector of the invention comprises a first strand that is substantially identical to a portion of a MIOX-like target gene of a plant genome and a second strand that is substantially complementary to the first strand. As shown in Example 1, the full length G. max MIOX-like gene was isolated and is represented in SEQ ID NO:27. The G. max MIOX-like gene sequence described by SEQ ID NO:27 contains an open reading frame with the amino acid sequence disclosed as SEQ ID NO:28. As shown in Example 3, the amino acid sequence described by SEQ ID NO:28 was used to identify homologous MIOX-like amino acid sequences from cotton, sugar beet, corn, and potato. The corresponding homologous amino acid sequences are set forth in SEQ ID NO:33, SEQ ID NO:35, SEQ ID NO:37, SEQ ID NO:39, and SEQ ID NO:41, respectively, and an alignment of the representative MIOX-like protein sequences or sequence fragments is shown in FIG. 11a-b. The corresponding homologous DNA sequences are described by SEQ ID NO:32, SEQ ID NO:34, SEQ ID NO:36, SEQ ID NO:38, and SEQ ID NO:40, and an alignment of the representative MIOX-like homologs with SEQ ID NO:27 is shown in FIG. 12a-e.

Accordingly, in this embodiment, the plant MIOX-like target gene is selected from the group consisting of: (a) a polynucleotide encoding a plant MIOX-like protein having at least 80% sequence identity to a plant MIOX-like protein having a sequence as set forth in SEQ ID NO:28, SEQ ID NO:33, SEQ ID NO:35, SEQ ID NO:37, SEQ ID NO:39, or SEQ ID NO:41 (b) a polynucleotide having a sequence as set forth in SEQ ID NO:27, SEQ ID NO:29, SEQ ID NO:30, SEQ ID NO:32, SEQ ID NO:34, SEQ ID NO:36, SEQ ID NO:38, or SEQ ID NO:40; (c) a polynucleotide having at least 80% sequence identity to SEQ ID NO:27, SEQ ID NO:29, SEQ ID NO:30, SEQ ID NO:32, SEQ ID NO:34, SEQ ID NO:36, SEQ ID NO:38, or SEQ ID NO:40 and (d) a polynucleotide from a parasitic nematode that hybridizes under stringent conditions to the sequence set forth in SEQ ID NO:27, SEQ ID NO:29, SEQ ID NO:30, SEQ ID NO:32, SEQ ID NO:34, SEQ ID NO:36, SEQ ID NO:38, or SEQ ID NO:40.

Additional cDNAs corresponding to the plant target genes of the invention may be isolated from plants other than G. max using the information provided herein and techniques known to those of skill in the art of biotechnology. For example, a nucleic acid molecule from a plant that hybridizes under stringent conditions to a nucleotide sequence of SEQ ID NO:1, 3, 4, 6, 7, 9, 11, 12, 14, 16, 18, 19, 21, 22, 24, 25, 27, 29, or 30 can be isolated from plant cDNA libraries. As used herein with regard to hybridization for DNA to a DNA blot, the term “stringent conditions” refers to hybridization overnight at 60° C. in 10×Denhart's solution, 6×SSC, 0.5% SDS, and 100 μg/ml denatured salmon sperm DNA. Blots are washed sequentially at 62° C. for 30 minutes each time in 3×SSC/0.1% SDS, followed by 1×SSC/0.1% SDS, and finally 0.1×SSC/0.1% SDS. As also used herein, in a preferred embodiment, the phrase “stringent conditions” refers to hybridization in a 6×SSC solution at 65° C. In another embodiment, “highly stringent conditions” refers to hybridization overnight at 65° C. in 10×Denhart's solution, 6×SSC, 0.5% SDS and 100 μg/ml denatured salmon sperm DNA. Blots are washed sequentially at 65° C. for 30 minutes each time in 3×SSC/0.1% SDS, followed by 1×SSC/0.1% SDS, and finally 0.1×SSC/0.1% SDS. Methods for nucleic acid hybridizations are described in Meinkoth and Wahl, 1984, Anal. Biochem. 138:267-284; well known in the art. Alternatively, mRNA can be isolated from plant cells, and cDNA can be prepared using reverse transcriptase. Synthetic oligonucleotide primers for polymerase chain reaction amplification can be designed based upon the nucleotide sequence shown in SEQ ID NO: 1, 3, 4, 6, 7, 9, 11, 12, 14, 16, 18, 19, 21, 22, 24, 25, 27, 29, or 30. Nucleic acid molecules corresponding to the plant target genes of the invention can be amplified using cDNA or, alternatively, genomic DNA, as a template and appropriate oligonucleotide primers according to standard PCR amplification techniques. The nucleic acid molecules so amplified can be cloned into appropriate vectors and characterized by DNA sequence analysis.

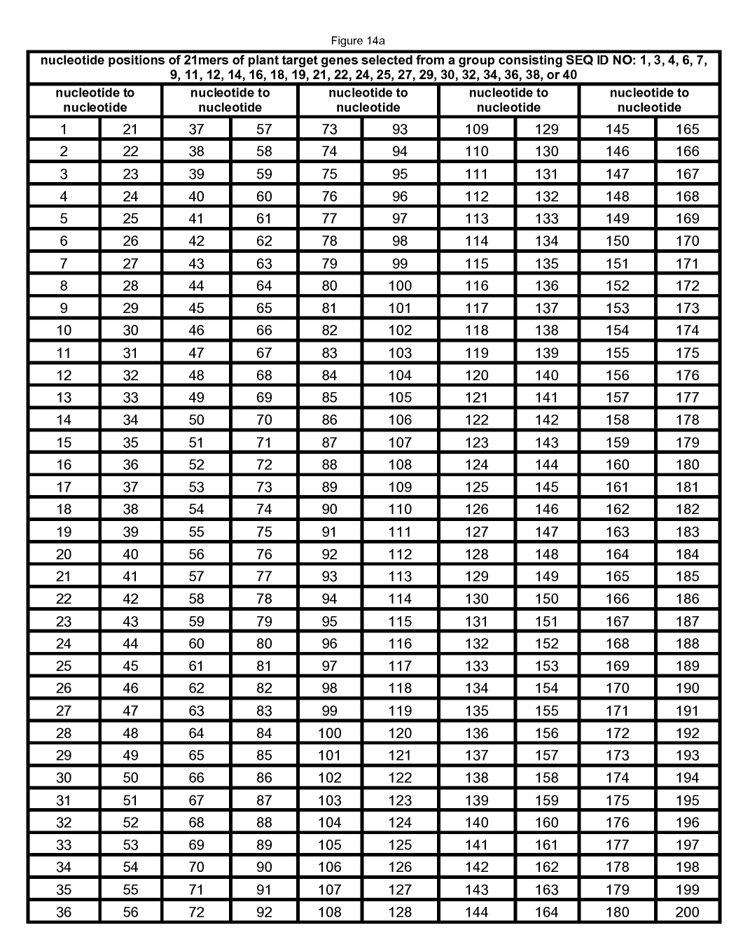

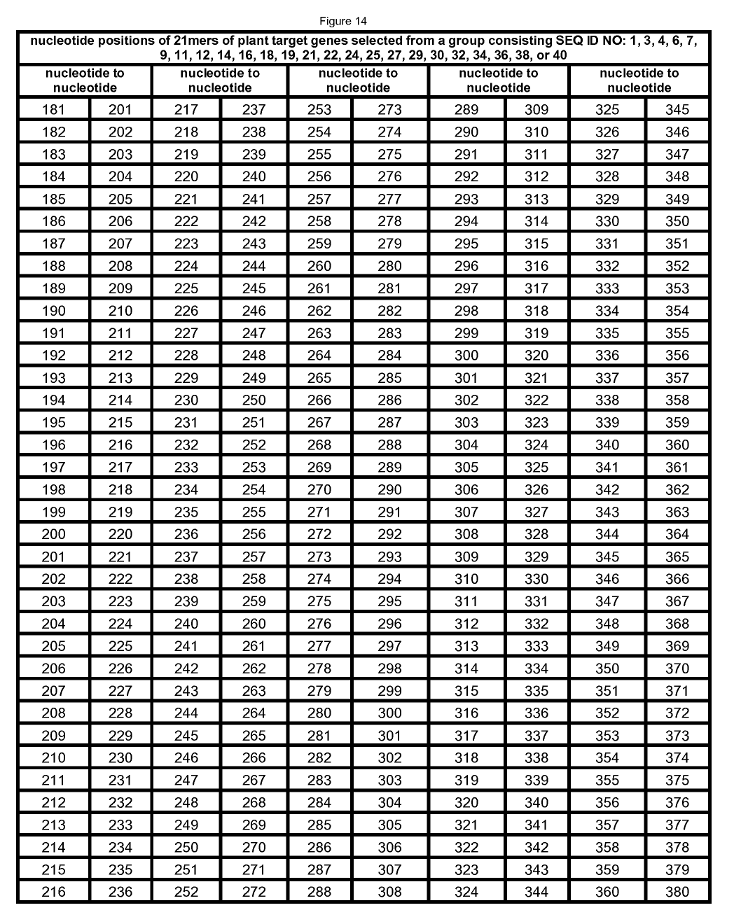

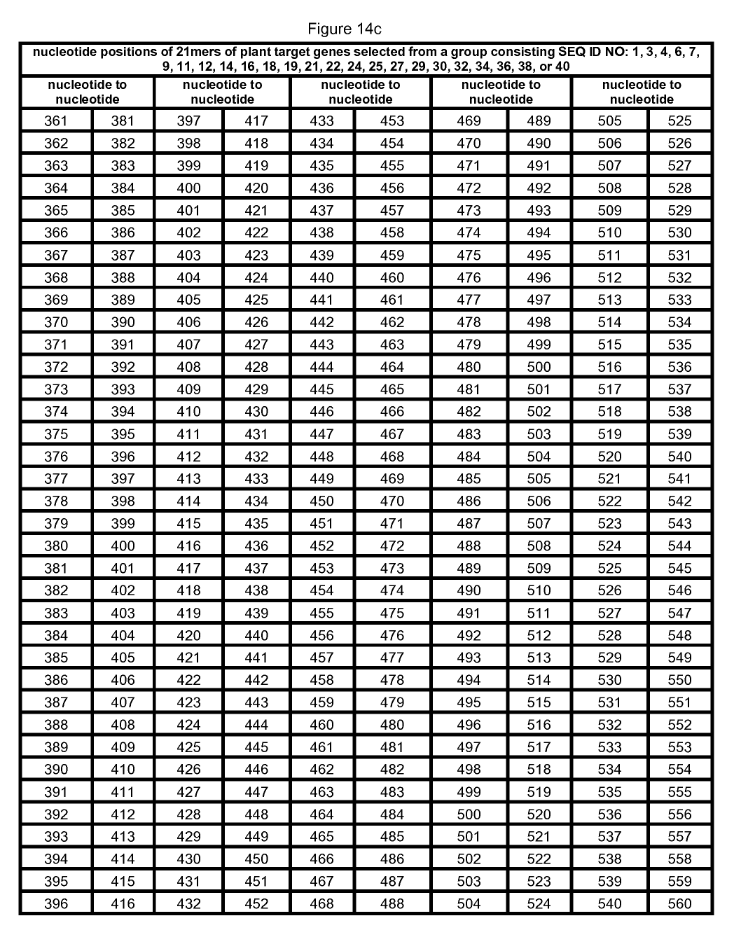

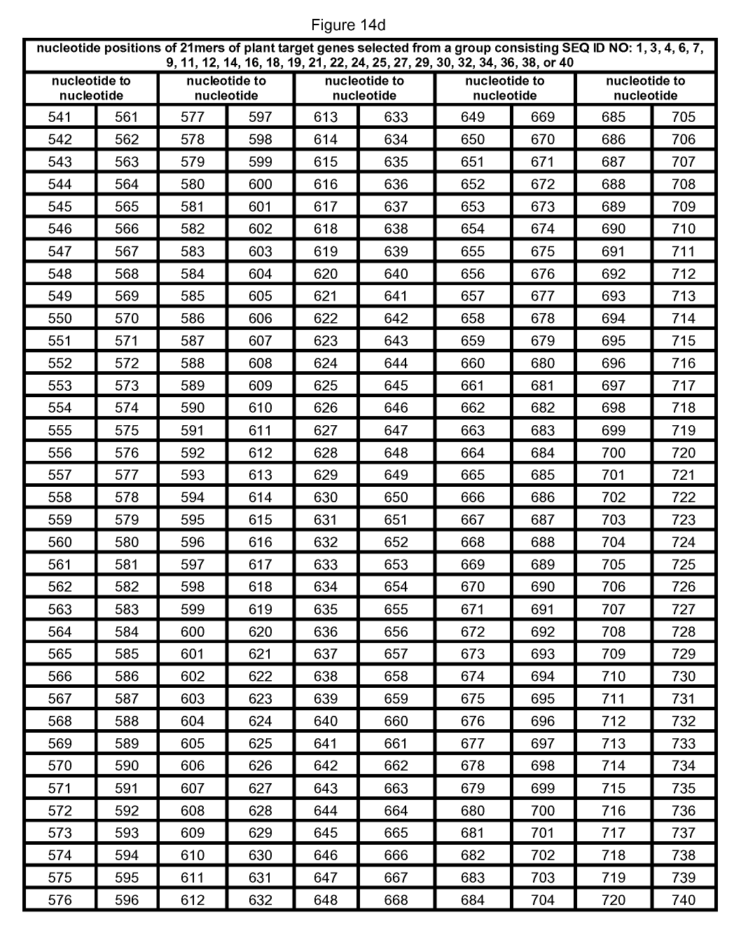

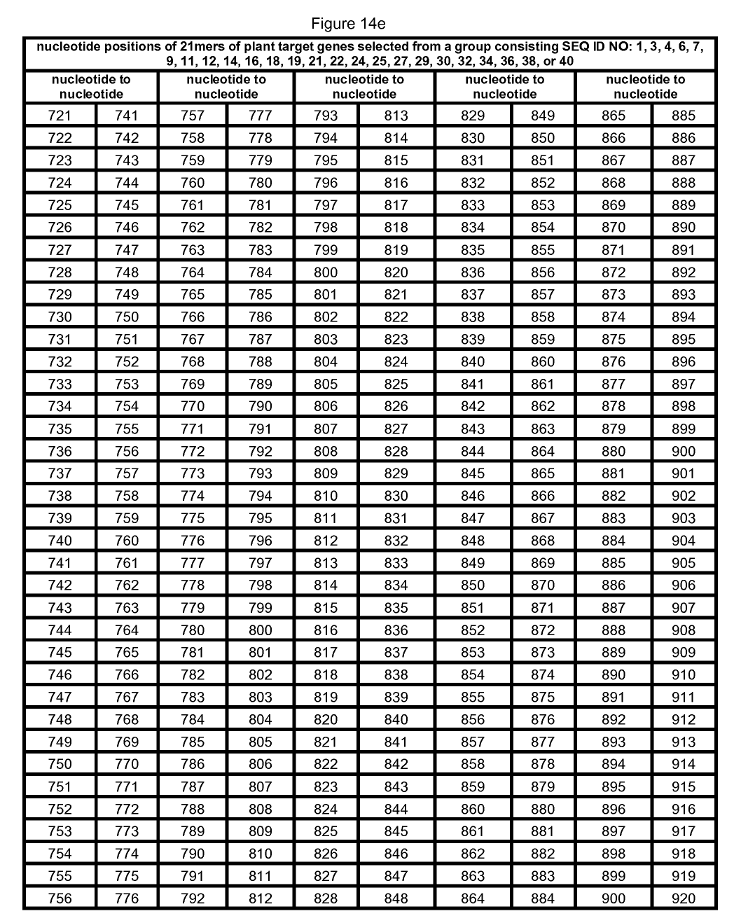

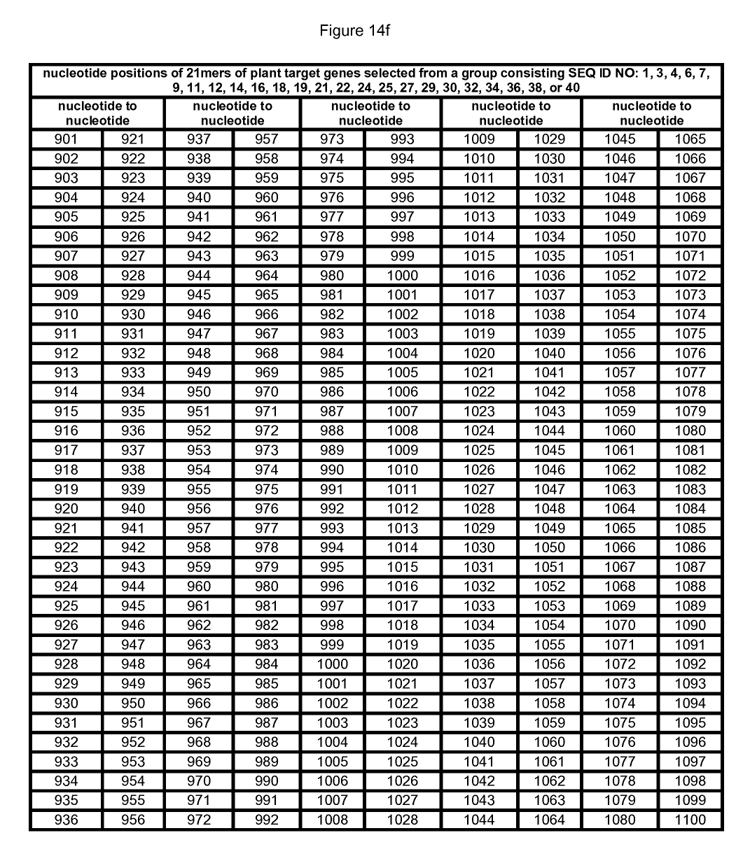

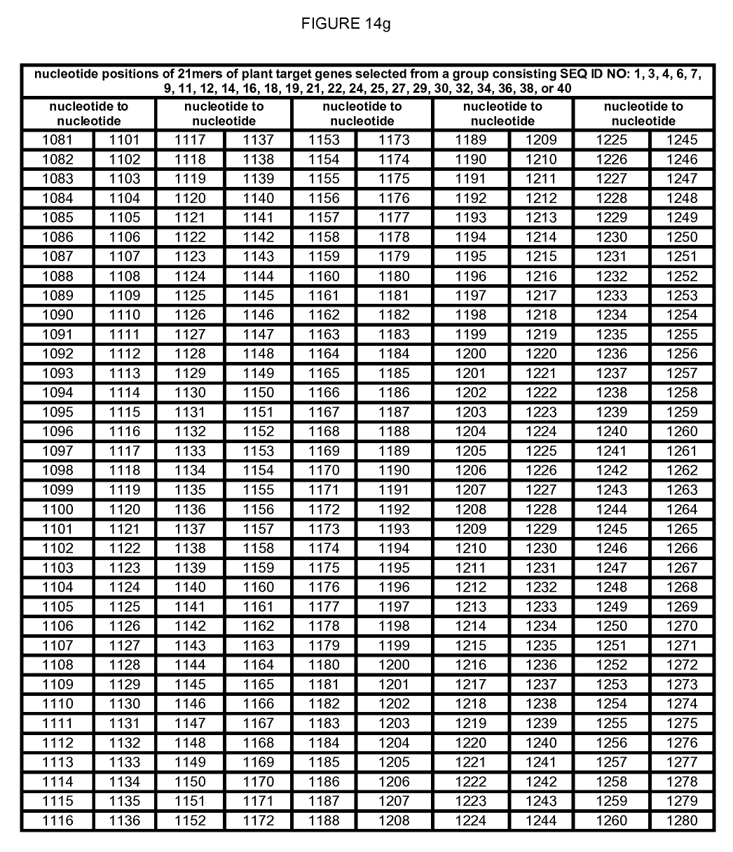

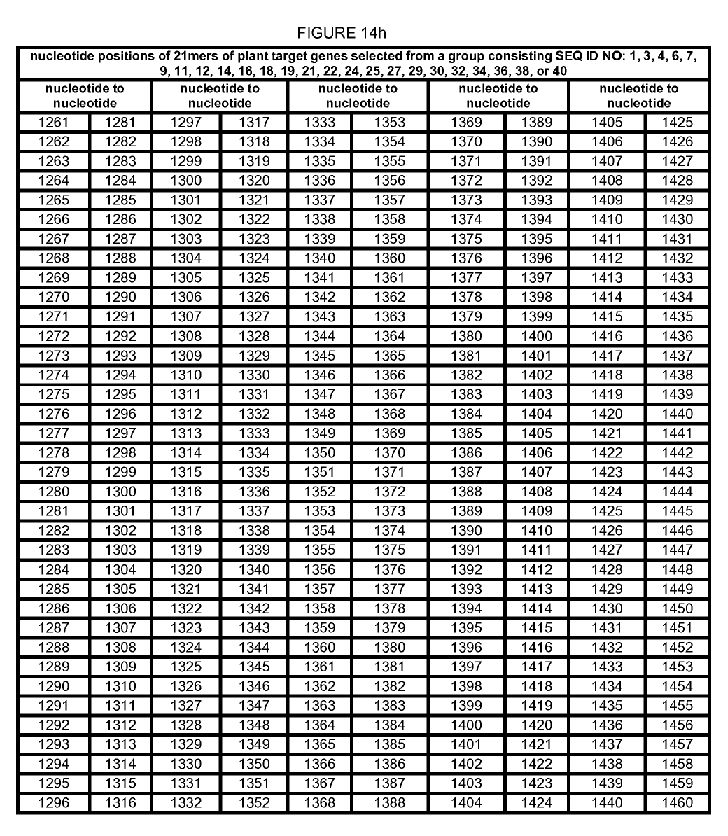

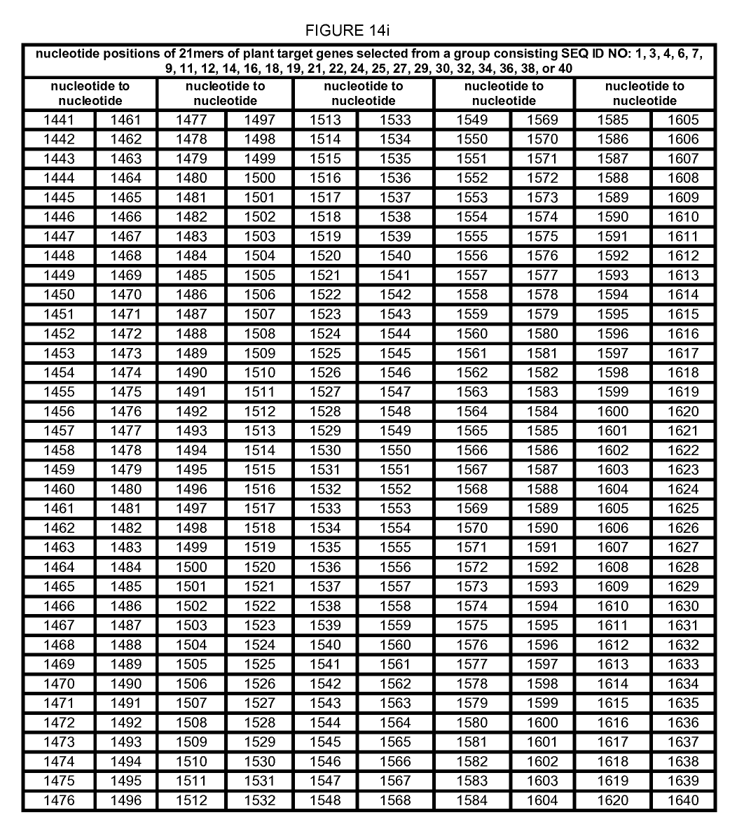

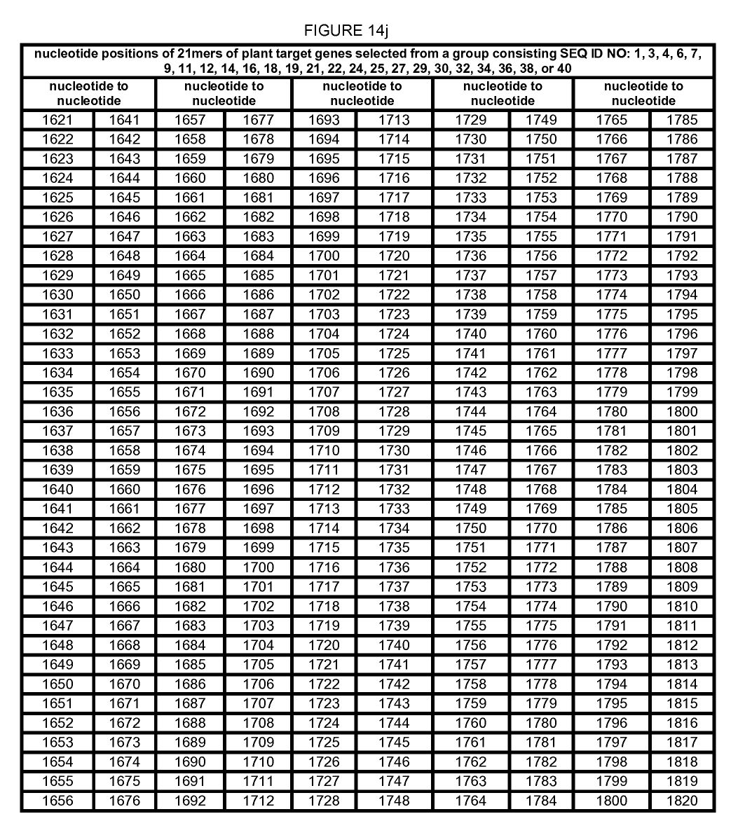

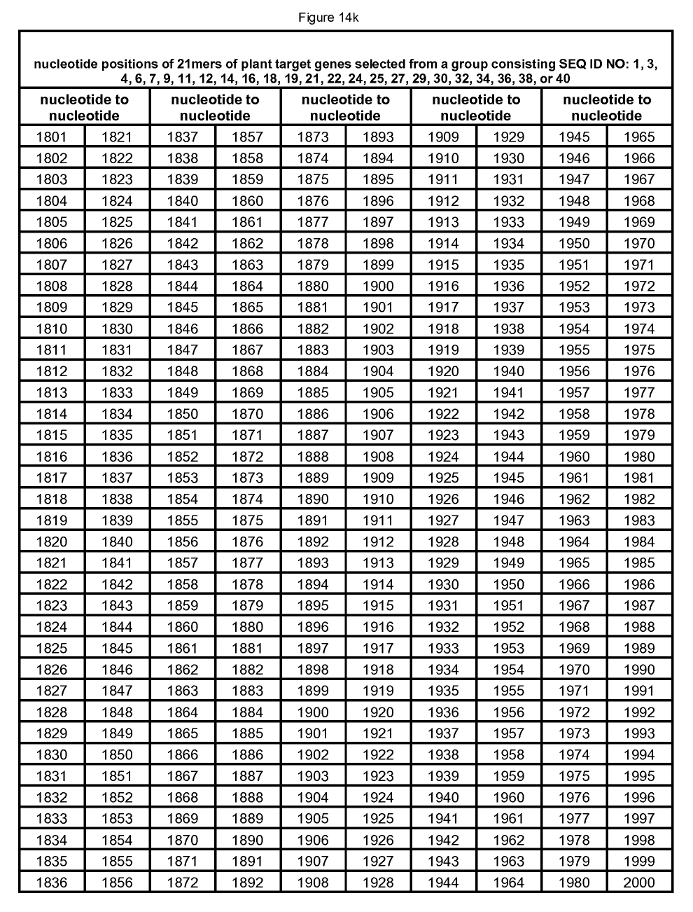

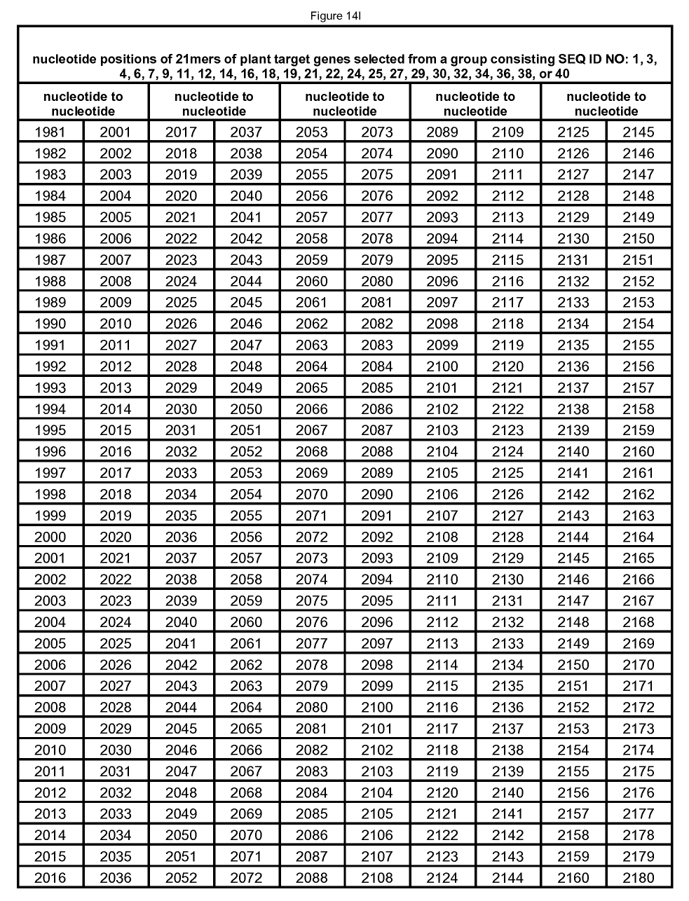

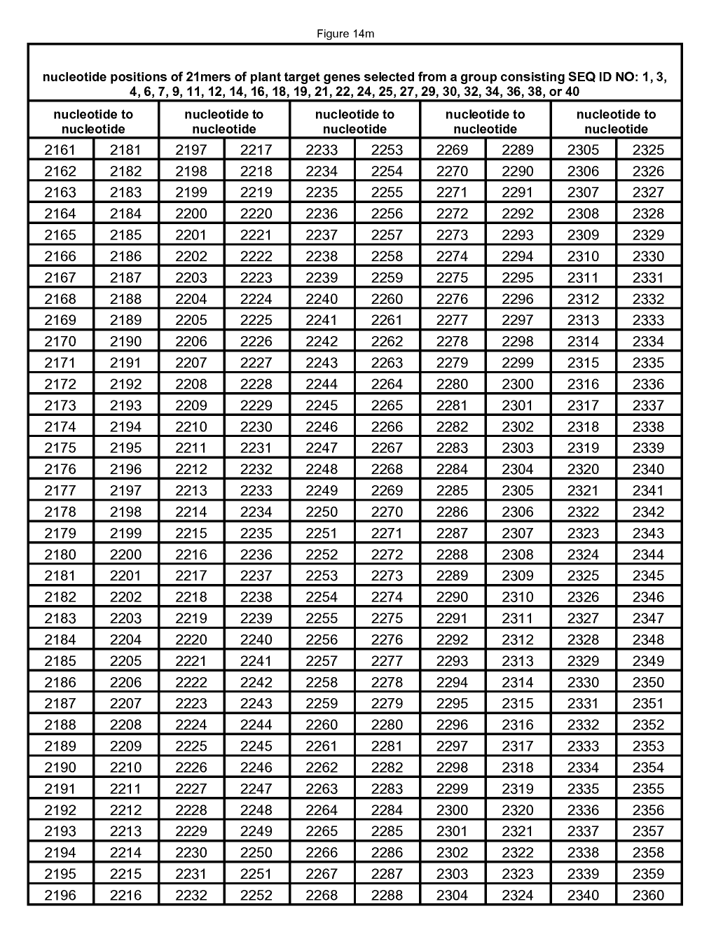

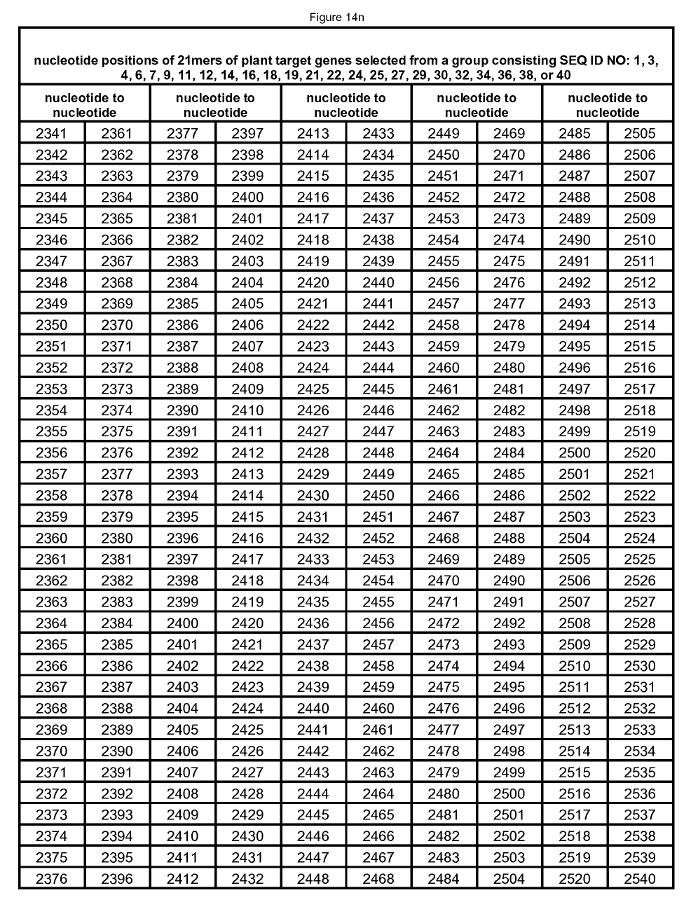

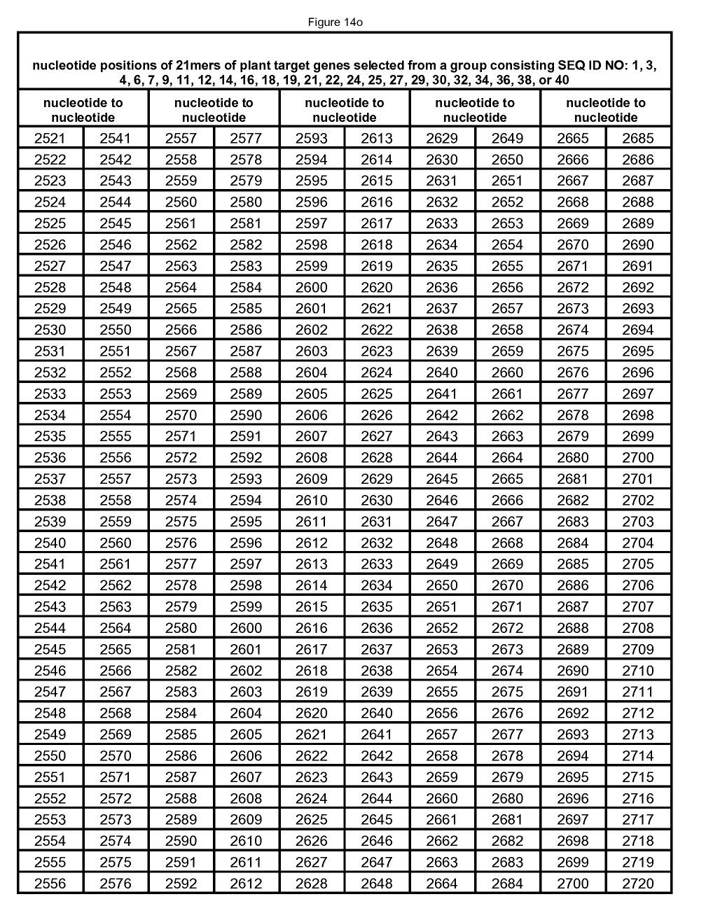

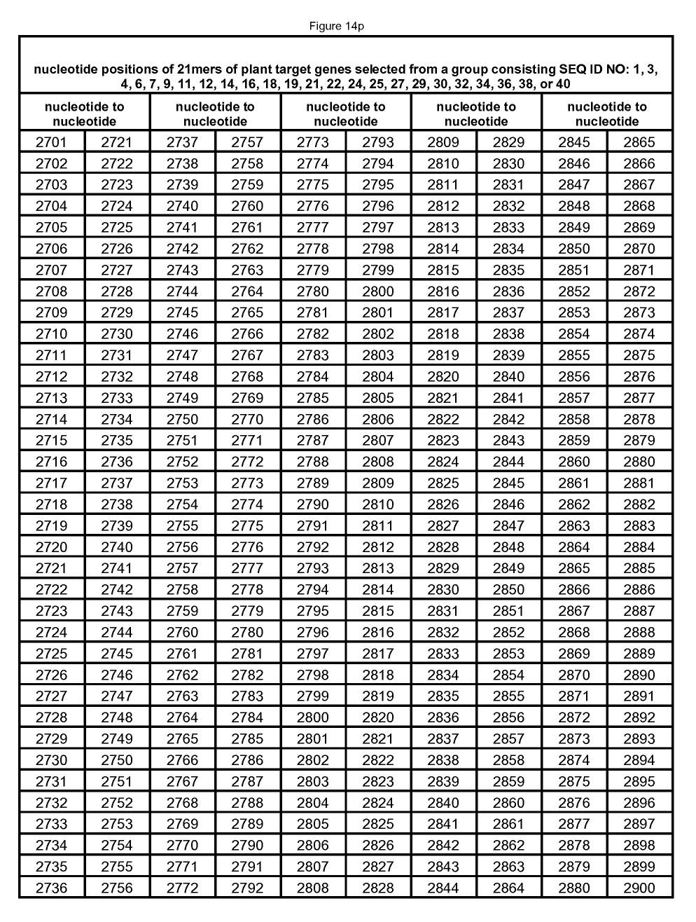

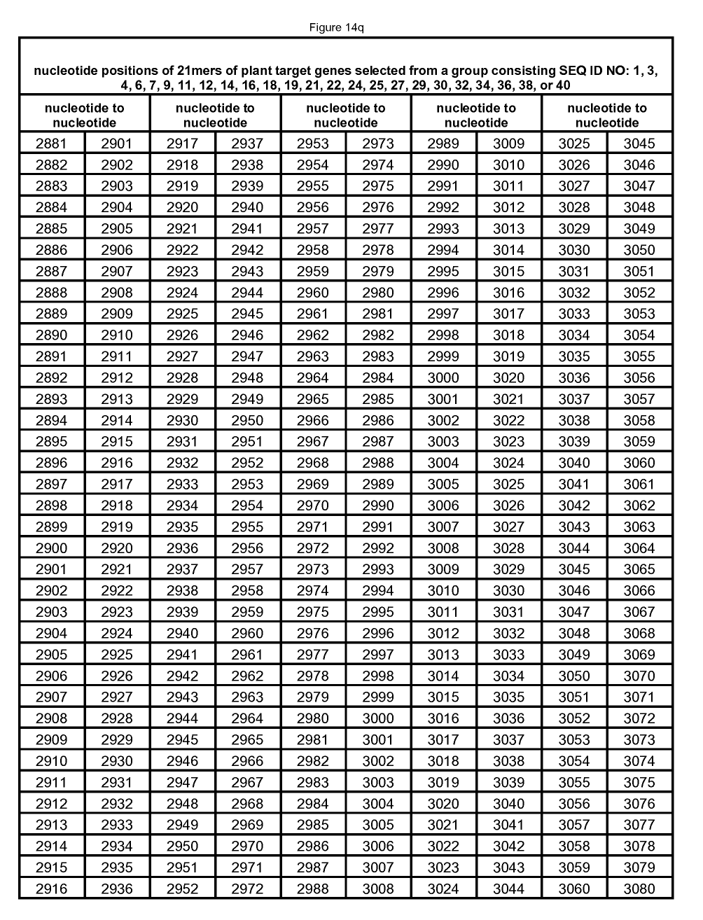

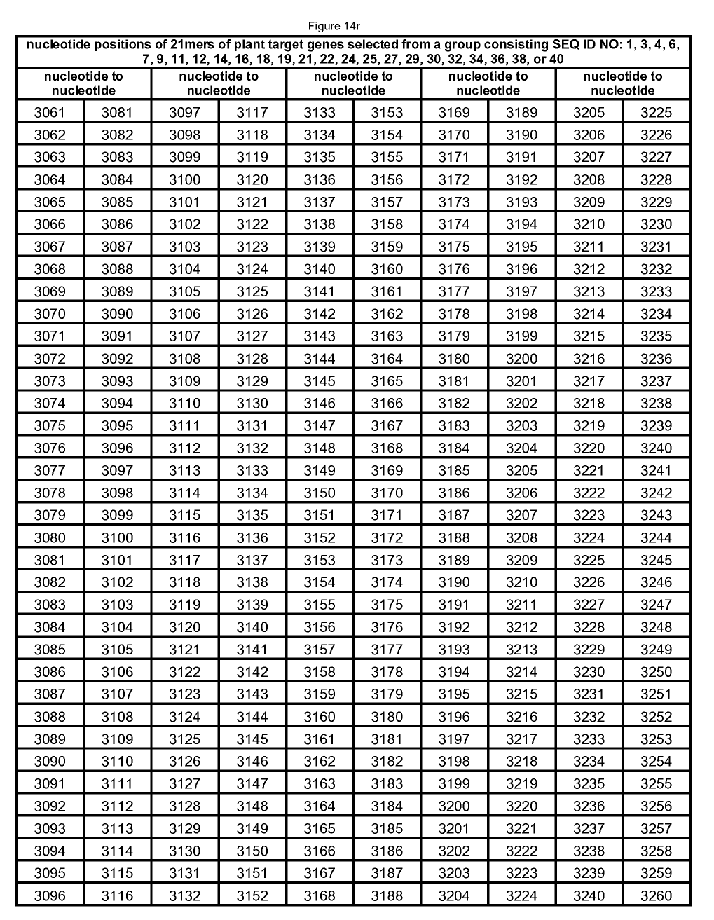

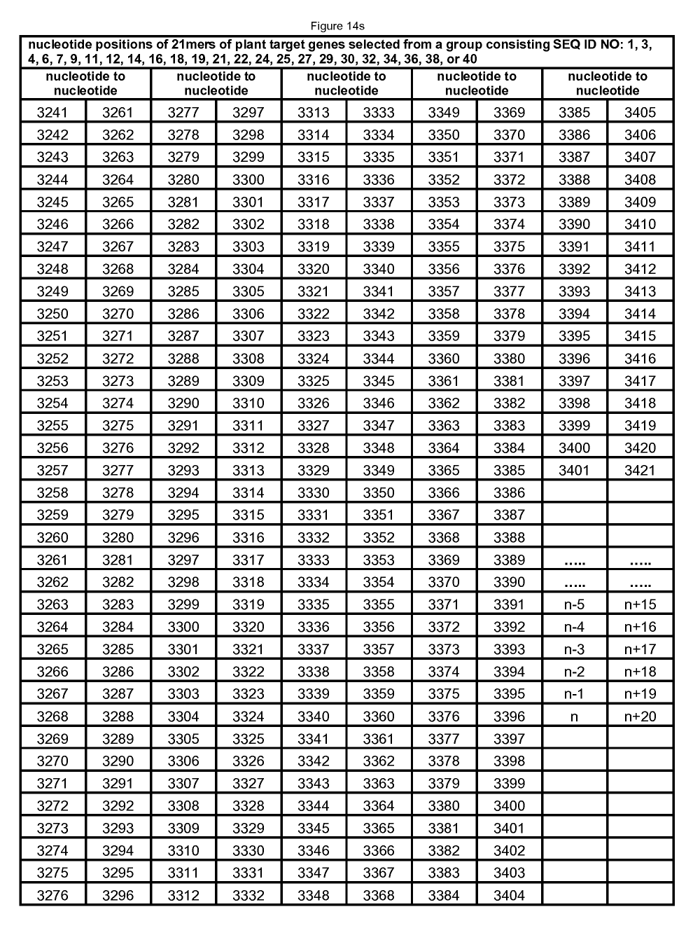

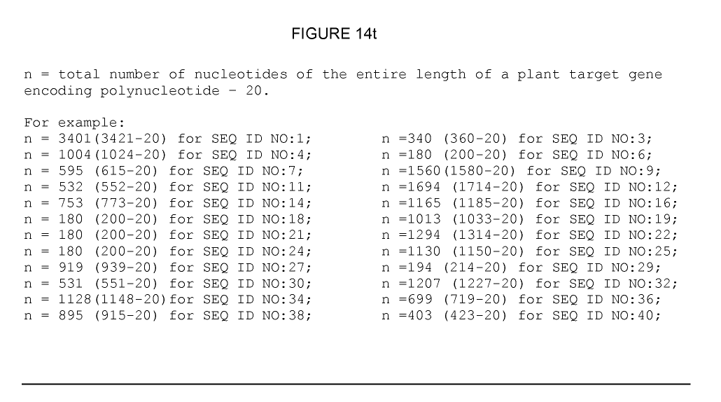

As discussed above, fragments of dsRNA larger than about 19-24 nucleotides in length are cleaved intracellularly by nematodes and plants to siRNAs of about 19-24 nucleotides in length, and these siRNAs are the actual mediators of the RNAi phenomenon. The table in FIGS. 14a-t sets forth exemplary 21-mers of the soybean GLABRA-like gene, SEQ ID NO:1, homeodomain-like gene, SEQ ID NO:4, trehalose-6-phosphate phosphatase-like gene, SEQ ID NO:9, unknown gene, SEQ ID NO:16, ringH2 finger-like gene, SEQ ID NO:19, zinc finger-like gene, SEQ ID NO:22, and the MIOX-like gene, SEQ ID NO:27 and the respective fragments and homologs thereof, as indicated by SEQ ID NOs set forth in the table. This table can also be used to calculate the 19, 20, 22, 23, or 24-mers by adding or subtracting the appropriate number of nucleotides from each 21 mer.

The expression vector of the invention encodes at least one dsRNA which may range in length from about 19 nucleotides to about 500 consecutive nucleotides or up to the whole length of the target gene. The dsRNA encoded by the expression vector of the invention may be embodied as a miRNA which targets a single site corresponding to a portion of the target gene comprising 19, 20, or 21 contiguous nucleotides thereof. Alternatively, the dsRNA encoded by the expression vector of the invention may have has a length from about 19, 20, or 21 nucleotides to about 600 consecutive nucleotides. In another embodiment, the dsRNA encoded by the expression vector of the invention has a length from about 19, 20, or 21 nucleotides to about 400 consecutive nucleotides, or from about 19, 20, or 21 nucleotides to about 300 consecutive nucleotides.

As disclosed herein, 100% sequence identity between the dsRNA and the target gene is not required to practice the present invention. Preferably, the dsRNA of the invention comprises a 19-nucleotide portion which is substantially identical to a 19 contiguous nucleotide portion of the target gene. While a dsRNA comprising a nucleotide sequence identical to a portion of the plant target genes of the invention is preferred for inhibition, the invention can tolerate sequence variations that might be expected due to gene manipulation or synthesis, genetic mutation, strain polymorphism, or evolutionary divergence. Thus the dsRNAs of the invention also encompass dsRNAs comprising a mismatch with the target gene of at least 1, 2, or more nucleotides. For example, it is contemplated in the present invention that the 21 mer dsRNA sequences exemplified in FIGS. 14a-14t may contain an addition, deletion or substitution of 1, 2, or more nucleotides, so long as the resulting sequence still interferes with the plant target gene function.

Sequence identity between the dsRNAs of the invention and the plant target genes may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 80% sequence identity, 90% sequence identity, or even 100% sequence identity, between the inhibitory RNA and at least 19 contiguous nucleotides of the target gene is preferred.

When the expression vector of the invention encodes a dsRNA having a length longer than about 21 nucleotides, for example, from about 50 nucleotides to about 1000 nucleotides, the encoded dsRNA will be cleaved randomly to siRNAs of about 19-24 nucleotides within the plant cell. The cleavage of a longer dsRNA of the invention will yield a pool of 19 mer, 20 mer, 21 mer, 22 mer, 23 mer or 24 mer dsRNAs, all of which are derived from the longer dsRNA. The siRNAs produced by the expression vectors of the invention have sequences corresponding to fragments of about 19-24 contiguous nucleotides across the entire sequence of the plant target gene. For example, a pool of siRNA produced by the expression vector of the invention derived from the target genes set forth in SEQ ID NO: 1, 3, 4, 6, 7, 9, 11, 12, 14, 16, 18, 19, 21, 22, 24, 25, 27, 29, 30, 32, 34, 36, 38, or 40 may comprise a multiplicity of RNA molecules which are selected from the group consisting of oligonucleotides substantially identical to the 21 mer nucleotides of SEQ ID NO: 1, 3, 4, 6, 7, 9, 11, 12, 14, 16, 18, 19, 21, 22, 24, 25, 27, 29, 30, 32, 34, 36, 38, or 40 found in FIGS. 14a-14t A pool of siRNA encoded by the expression vector of the invention may also comprise any combination of the specific RNA molecules having any of the 21 contiguous nucleotide sequences derived from SEQ ID NO: 1, 3, 4, 6, 7, 9, 11, 12, 14, 16, 18, 19, 21, 22, 24, 25, 27, 29, 30, 32, 34, 36, 38, or 40 set forth in FIGS. 14a-14t. Further, as multiple specialized Dicers in plants generate siRNAs typically ranging in size from 19 nt to 24 nt (See Henderson et al., 2006. Nature Genetics 38:721-725.), the siRNAs encoded by the expression vector of the present invention can may range from about 19 contiguous nucleotides to about 24 contiguous nucleotides derived from. Similarly, a pool of siRNA encoded by the expression vector of the invention may comprise a multiplicity of RNA molecules having any 19, 20, 21, 22, 23, or 24 contiguous nucleotide sequences derived from SEQ ID NO: 1, 3, 4, 6, 7, 9, 11, 12, 14, 16, 18, 19, 21, 22, 24, 25, 27, 29, 30, 32, 34, 36, 38, or 40. Alternatively, the pool of siRNA encoded by the expression vector of the invention may comprise a multiplicity of RNA molecules having a combination of any 19, 20, 21, 22, 23, and/or 24 contiguous nucleotide sequences derived from SEQ ID NO: 1, 3, 4, 6, 7, 9, 11, 12, 14, 16, 18, 19, 21, 22, 24, 25, 27, 29, 30, 32, 34, 36, 38, or 40.

The expression vector of the invention may optionally encode a dsRNA which comprises a single stranded overhang at either or both ends. Preferably, the single stranded overhang comprises at least two nucleotides at the 3′ end of each strand of the dsRNA molecule. The double-stranded structure may be formed by a single self-complementary RNA strand (i.e. forming a hairpin loop) or two complementary RNA strands. RNA duplex formation may be initiated either inside or outside the cell. When the dsRNA of the invention forms a hairpin loop, it may optionally comprise an intron, as set forth in US 2003/0180945A1 or a nucleotide spacer, which is a stretch of sequence between the complementary RNA strands to stabilize the hairpin transgene in cells. Methods for making various dsRNA molecules are set forth, for example, in WO 99/53050 and in U.S. Pat. No. 6,506,559. The RNA may be introduced in an amount that allows delivery of at least one copy per cell. Higher doses of double-stranded material may yield more effective inhibition.

As described above, the isolated expression vector of the invention comprises a nucleic acid encoding a dsRNA molecule, wherein expression of the vector in a host plant cell results in increased resistance to a parasitic nematode as compared to a wild-type variety of the host plant cell. The isolated expression vectors of the invention is capable of mediating expression of the encoded dsRNA in a host plant cell, which means that the recombinant expression vector includes one or more regulatory sequences, e.g. promoters, selected on the basis of the host plant cells to be used for expression, which is operatively linked to the nucleic acid encoding the dsRNA. In one embodiment, the nucleic acid molecule further comprises a promoter flanking either end of the nucleic acid molecule, wherein the promoters drive expression of each individual DNA strand, thereby generating two complementary RNAs that hybridize and form the dsRNA. In another embodiment, the nucleic acid molecule comprises a nucleotide sequence that is transcribed into both strands of the dsRNA on one transcription unit, wherein the sense strand is transcribed from the 5′ end of the transcription unit and the antisense strand is transcribed from the 3′ end, wherein the two strands are separated by 3 to 500 base or more pairs, and wherein after transcription, the RNA transcript folds on itself to form a hairpin. In accordance with the invention, the spacer region in the hairpin transcript may be any DNA fragment.

According to the present invention, the introduced polynucleotide may be maintained in the plant cell stably if it is incorporated into a non-chromosomal autonomous replicon or integrated into the plant chromosomes. Alternatively, the introduced polynucleotide may be present on an extra-chromosomal non-replicating vector and be transiently expressed or transiently active. Whether present in an extra-chromosomal non-replicating vector or a vector that is integrated into a chromosome, the polynucleotide preferably resides in a plant expression cassette. A plant expression cassette preferably contains regulatory sequences capable of driving gene expression in plant cells that are operatively linked so that each sequence can fulfill its function, for example, termination of transcription by polyadenylation signals. Preferred polyadenylation signals are those originating from Agrobacterium tumefaciens t-DNA such as the gene 3 known as octopine synthase of the Ti-plasmid pTiACH5 (Gielen et al., 1984, EMBO J. 3:835) or functional equivalents thereof, but also all other terminators functionally active in plants are suitable. As plant gene expression is very often not limited on transcriptional levels, a plant expression cassette preferably contains other operatively linked sequences like translational enhancers such as the overdrive-sequence containing the 5′-untranslated leader sequence from tobacco mosaic virus enhancing the polypeptide per RNA ratio (Gallie et al., 1987, Nucl. Acids Research 15:8693-8711). Examples of plant expression vectors include those detailed in: Becker, D. et al., 1992, New plant binary vectors with selectable markers located proximal to the left border, Plant Mol. Biol. 20:1195-1197; Bevan, M. W., 1984, Binary Agrobacterium vectors for plant transformation, Nucl. Acid. Res. 12:8711-8721; and Vectors for Gene Transfer in Higher Plants; in: Transgenic Plants, Vol. 1, Engineering and Utilization, eds.: Kung and R. Wu, Academic Press, 1993, S. 15-38.

Promoters useful in the expression cassette of the invention include any promoter that is capable of initiating transcription in a plant cell present in the plant's roots. Such promoters include, but are not limited to, those that can be obtained from plants, plant viruses and bacteria that contain genes that are expressed in plants, such as Agrobacterium and Rhizobium. Promoters capable of expressing the encoded dsRNA in a cell that is contacted by parasitic nematodes are preferred. Alternatively, the promoter may drive expression of the dsRNA in a plant tissue remote from the site of contact with the nematode, and the dsRNA may then be transported by the plant to a cell that is contacted by the parasitic nematode, in particular cells of, or close by nematode feeding sites, e.g. syncytial cells or giant cells. Preferably, the expression cassette of the invention comprises a root-specific promoter, a pathogen inducible promoter, or a nematode inducible promoter. More preferably the nematode inducible promoter is a parasitic nematode feeding site-specific promoter. A parasitic nematode feeding site-specific promoter may be specific for syncytial cells or giant cells or specific for both kinds of cells. Of particular utility in the present invention are syncytia site preferred, or nematode feeding site induced, promoters, including, but not limited to promoters from the Mtn3-like promoter disclosed in commonly owned copending WO 2008/095887, the Mtn21-like promoter disclosed in commonly owned copending WO 2007/096275, the peroxidase-like promoter disclosed in commonly owned copending WO 2008/077892, the trehalose-6-phosphate phosphatase-like promoter disclosed in commonly owned copending WO 2008/071726 and the At5g12170-like promoter disclosed in commonly owned copending WO 2008/095888. All of the forgoing applications are incorporated herein by reference.

In addition, the promoters TobRB7, AtRPE, AtPyk10, Geminil9, and AtHMG1 have been shown to be induced by nematodes (for a review of nematode-inducible promoters, see Ann. Rev. Phytopathol. (2002) 40:191-219; see also U.S. Pat. No. 6,593,513). Methods for isolating additional nematode-inducible promoters are set forth in U.S. Pat. Nos. 5,589,622 and 5,824,876. Plant gene expression can also be facilitated via an inducible promoter (For review, see Gatz, 1997, Annu. Rev. Plant Physiol. Plant Mol. Biol. 48:89-108). Other inducible promoters include the hsp80 promoter from Brassica, being inducible by heat shock; the PPDK promoter is induced by light; the PR-1 promoter from tobacco, Arabidopsis, and maize are inducible by infection with a pathogen; and the Adh1 promoter is induced by hypoxia and cold stress. Chemically inducible promoters are especially suitable if time-specific gene expression is desired. Non-limiting examples of such promoters are a salicylic acid inducible promoter (PCT Application No. WO 95/19443), a tetracycline inducible promoter (Gatz et al., 1992, Plant J. 2:397-404) and an ethanol inducible promoter (PCT Application No. WO 93/21334).

Alternatively, the promoter may be constitutive, developmental stage-preferred, cell type-preferred, tissue-preferred or organ-preferred. Constitutive promoters are active under most conditions. Non-limiting examples of constitutive promoters include the CaMV 19S and 35S promoters (Odell et al., 1985, Nature 313:810-812), the sX CaMV 35S promoter (Kay et al., 1987, Science 236:1299-1302), the Sep1 promoter, the rice actin promoter (McElroy et al., 1990, Plant Cell 2:163-171), the Arabidopsis actin promoter, the ubiquitin promoter (Christensen et al., 1989, Plant Molec. Biol. 18:675-689); pEmu (Last et al., 1991, Theor. Appl. Genet. 81:581-588), the figwort mosaic virus 35S promoter, the Smas promoter (Velten et al., 1984, EMBO J. 3:2723-2730), the GRP1-8 promoter, the cinnamyl alcohol dehydrogenase promoter (U.S. Pat. No. 5,683,439), promoters from the T-DNA of Agrobacterium, such as mannopine synthase, nopaline synthase, and octopine synthase, the small subunit of ribulose biphosphate carboxylase (ssuRUBISCO) promoter, and the like.

In another embodiment, the expression vector of the invention vector comprises a bidirectional promoter, driving expression of two nucleic acid molecules, whereby one nucleic acid molecule codes for a sequence substantially identical to the first strand of a dsRNA that is substantially identical to a plant target gene selected from the group consisting of the GLABRA-like gene, homeodomain-like gene, trehalose-6-phosphate phosphatase-like gene, unknown gene, ringH2 finger-like gene, zinc finger-like gene, or MIOX-like gene described herein, and the other nucleic acid molecule codes for the second strand of the dsRNA that is complementary to the first strand, wherein the two strands are capable of forming a dsRNA when both sequences are transcribed. A bidirectional promoter is a promoter capable of mediating expression in two directions. Alternatively, the expression vector of the invention comprises two promoters, the first promoter mediating transcription of the first strand of a dsRNA that is substantially identical to a portion of a plant target gene selected from the group consisting of the GLABRA-like gene, homeodomain-like gene, trehalose-6-phosphate phosphatase-like gene, unknown gene, ringH2 finger-like gene, zinc finger-like gene, or MIOX-like gene described herein, and the second promoter mediating transcription of the second strand of the dsRNA that is complementary to the first strand and capable of forming a dsRNA, when both sequences are transcribed. For example, the first promoter may be constitutive or tissue specific and the second promoter may be tissue specific or inducible by pathogens.

The invention is also embodied in a transgenic plant comprising the expression vector of the invention. The transgenic plant of this embodiment is capable of expressing the dsRNA described above and thereby inhibiting the GLABRA-like target gene, homeodomain-like target gene, trehalose-6-phosphate phosphatase-like target gene, unknown target gene, ringH2 finger-like target gene, zinc finger-like target gene, or MIOX-like target gene. The transgenic plant of this embodiment is thus nematode resistant.

In accordance with the invention, the plant is a monocotyledonous plant or a dicotyledonous plant. The transgenic plant of the invention may be of any species that is susceptible to infection by plant parasitic nematodes, such species including, without limitation, Medicago, Solanum, Brassica, Cucumis, Juglans, Gossypium, Malus, Vitis, Antirrhinum, Populus, Fragaria, Arabidopsis, Picea, Capsicum, Chenopodium, Dendranthema, Pharbitis, Pinus, Pisum, Oryza, Zea, Triticum, Triticale, Secale, Lolium, Hordeum, Glycine, Pseudotsuga, Kalanchoe, Beta, Helianthus, Nicotiana, Cucurbita, Rosa, Fragaria, Lotus, Onobrychis, trifolium, Trigonella, Vigna, Citrus, Linum, Geranium, Manihot, Daucus, Raphanus, Sinapis, Atropa, Datura, Hyoscyamus, Petunia, Digitalis, Majorana, Ciahorium, Lactuca, Bromus, Asparagus, Antirrhinum, Heterocallis, Nemesis, Pelargonium, Panieum, Pennisetum, Ranunculus, Senecio, Salpiglossis, Browaalia, Phaseolus, Avena, and Allium. Preferably the plant is a crop plant such as wheat, barley, sorghum, rye, triticale, maize, rice, sugarcane, pea, alfalfa, soybean, carrot, celery, tomato, potato, cotton, tobacco, pepper, canola, oilseed rape, beet, cabbage, cauliflower, broccoli, or lettuce.

Any method may be used to transform the expression vector of the invention into plant cells to yield the transgenic plants of the invention. Suitable methods for transforming or transfecting host cells including plant cells are well known in the art of plant biotechnology. General methods for transforming dicotyledenous plants are disclosed, for example, in U.S. Pat. Nos. 4,940,838; 5,464,763, and the like. Methods for transforming specific dicotyledenous plants, for example, cotton, are set forth in U.S. Pat. Nos. 5,004,863; 5,159,135; and 5,846,797. Soybean transformation methods are set forth in U.S. Pat. Nos. 4,992,375; 5,416,011; 5,569,834; 5,824,877; 6,384,301 and in EP 0301749B1 may be used. Transformation methods may include direct and indirect methods of transformation. Suitable direct methods include polyethylene glycol induced DNA uptake, liposome-mediated transformation (U.S. Pat. No. 4,536,475), biolistic methods using the gene gun (Fromm M E et al., Bio/Technology. 8(9):833-9, 1990; Gordon-Kamm et al. Plant Cell 2:603, 1990), electroporation, incubation of dry embryos in DNA-comprising solution, and microinjection. If intact plants are to be regenerated from the transformed cells, an additional selectable marker gene is preferably located on the plasmid. The direct transformation techniques are equally suitable for dicotyledonous and monocotyledonous plants.

Transformation can also be carried out by bacterial infection by means of Agrobacterium (for example EP 0 116 718), viral infection by means of viral vectors (EP 0 067 553; U.S. Pat. No. 4,407,956; WO 95/34668; WO 93/03161) or by means of pollen (EP 0 270 356; WO 85/01856; U.S. Pat. No. 4,684,611). Agrobacterium based transformation techniques (especially for dicotyledonous plants) are well known in the art. The Agrobacterium strain (e.g., Agrobacterium tumefaciens or Agrobacterium rhizogenes) comprises a plasmid (Ti or Ri plasmid) and a T-DNA element which is transferred to the plant following infection with Agrobacterium. The T-DNA (transferred DNA) is integrated into the genome of the plant cell. The T-DNA may be localized on the Ri- or Ti-plasmid or is separately comprised in a so-called binary vector. Methods for the Agrobacterium-mediated transformation are described, for example, in Horsch RB et al. (1985) Science 225:1229. The Agrobacterium-mediated transformation is best suited to dicotyledonous plants but has also been adapted to monocotyledonous plants. The transformation of plants by Agrobacteria is described in, for example, White F F, Vectors for Gene Transfer in Higher Plants, Transgenic Plants, Vol. 1, Engineering and Utilization, edited by S. D. Kung and R. Wu, Academic Press, 1993, pp. 15-38; Jenes B et al. Techniques for Gene Transfer, Transgenic Plants, Vol. 1, Engineering and Utilization, edited by S. D. Kung and R. Wu, Academic Press, 1993, pp. 128-143; Potrykus (1991) Annu Rev Plant Physiol Plant Molec Biol 42:205-225. Transformation may result in transient or stable transformation and expression.

The transgenic plants of the invention may be crossed with similar transgenic plants or with transgenic plants lacking the nucleic acids of the invention or with non-transgenic plants, using known methods of plant breeding, to prepare seeds. Further, the transgenic plant of the present invention may comprise, and/or be crossed to another transgenic plant that comprises one or more nucleic acids, thus creating a “stack” of transgenes in the plant and/or its progeny. The seed is then planted to obtain a crossed fertile transgenic plant comprising the nucleic acid of the invention. The crossed fertile transgenic plant may have the particular expression cassette inherited through a female parent or through a male parent. The second plant may be an inbred plant. The crossed fertile transgenic may be a hybrid. Also included within the present invention are seeds of any of these crossed fertile transgenic plants. The seeds of this invention can be harvested from fertile transgenic plants and be used to grow progeny generations of transformed plants of this invention including hybrid plant lines comprising the DNA construct.

“Gene stacking” can also be accomplished by transferring two or more genes into the cell nucleus by plant transformation. Multiple genes may be introduced into the cell nucleus during transformation either sequentially or in unison. Multiple genes in plants or target pathogen species can be down-regulated by gene silencing mechanisms, specifically RNAi, by using a single transgene targeting multiple linked partial sequences of interest. Stacked, multiple genes under the control of individual promoters can also be over-expressed to attain a desired single or multiple phenotype. Constructs containing gene stacks of both over-expressed genes and silenced targets can also be introduced into plants yielding single or multiple agronomically important phenotypes. In these stacked embodiments, the expression vector of the invention further comprises nucleic acid sequences encoding traits other than the nematode-resistance encoding sequences described herein. In accordance with the invention, the dsRNA-encoding sequences of the expression vector can be stacked with any combination of polynucleotide sequences of interest to create desired phenotypes. The combinations can produce plants with a variety of trait combinations including but not limited to disease resistance, herbicide tolerance, yield enhancement, cold and drought tolerance. These stacked combinations can be created by any method including but not limited to cross breeding plants by conventional methods or by genetic transformation. If the traits are stacked by genetic transformation, the polynucleotide sequences of interest can be combined sequentially or simultaneously in any order. For example if two genes are to be introduced, the two sequences can be contained in separate transformation cassettes or on the same transformation cassette. The expression of the sequences can be driven by the same or different promoters.

In another embodiment, the invention provides a method the transgenic plant of the invention. This embodiment of the invention comprises the steps of, first, preparing an expression vector comprising a nucleic acid encoding the dsRNAs described above. In the second step of this method, the expression vector is transformed into a recipient plant. In the third step of this embodiment, one or more transgenic offspring of the transformed recipient plant is products. In the fourth step of this embodiment, nematode-resistant transgenic offspring are selected. Testing for nematode resistance may be performed, for example, using a hairy root assay or the rooted explant assay described in U.S. Pat. Pub. 2008/0153102, by field testing the transgenic offspring for nematode resistance, or by any other method of testing plants for nematode resistance.

As increased resistance to nematode infection is a general trait wished to be inherited into a wide variety of plants. Increased resistance to nematode infection is a general trait wished to be inherited into a wide variety of plants. The present invention may be used to reduce crop destruction by any plant parasitic nematode. Preferably, the parasitic nematodes belong to nematode families inducing giant or syncytial cells, such as Longidoridae, Trichodoridae, Heterodidae, Meloidogynidae, Pratylenchidae or Tylenchulidae. In particular in the families Heterodidae and Meloidogynidae. When the parasitic nematodes are of the genus Globodera, exemplary targeted species include, without limitation, G. achilleae, G. artemisiae, G. hypolysi, G. mexicana, G. millefolii, G. mali, G. pallida, G. rostochiensis, G. tabacum, and G. virginiae. When the parasitic nematodes are of the genus Heterodera, exemplary targeted species include, without limitation, H. avenae, H. carotae, H. ciceri, H. cruciferae, H. delvii, H. elachista, H. filipjevi, H. gambiensis, H. glycines, H. goettingiana, H. graduni, H. humuli, H. hordecalis, H. latipons, H. major, H. medicaginis, H. oryzicola, H. pakistanensis, H. rosii, H. sacchari, H. schachtii, H. sorghi, H. trifolii, H. urticae, H. vigni and H. zeae. When the parasitic nematodes are of the genus Meloidogyne, exemplary targeted species include, without limitation, M. acronea, M. arabica, M. arenaria, M. artiellia, M. brevicauda, M. camelliae, M. chitwoodi, M. cofeicola, M. esigua, M. graminicola, M. hapla, M. incognita, M. indica, M. inornata, M. javanica, M. lini, M. mali, M. microcephala, M. microtyla, M. naasi, M. salasi and M. thamesi.

The following examples are not intended to limit the scope of the claims to the invention, but are rather intended to be exemplary of certain embodiments. Any variations in the exemplified methods that occur to the skilled artisan are intended to fall within the scope of the present invention.

### Example 1

### Cloning of Target Genes and Vector Construction

Using available cDNA clone sequence for the soybean target genes, PCR was used to isolate DNA fragments approximately 200-500 bp in length that were used to construct the binary vectors described in Table 1 and discussed in Example 2. The PCR products were cloned into TOPO pCR2.1 vector (Invitrogen, Carlsbad, Calif.) and inserts were confirmed by sequencing. Gene fragments for the target genes GmTPP-like, GmGLABRA-like, and GmMIOX-like were isolated using this method. Alternatively, available cDNA clone sequence for the soybean target gene was used to identify DNA fragments approximately 200-300 bp in length that were used to construct the binary vectors described in Table 1 and discussed in Example 2. The identified DNA sequences for the soybean target genes were synthesized, cloned into a pUC19 (Invitrogen) vector, and verified by sequencing. Gene fragments for the target genes GmHD-like, GmRingH2 Finger-like, GmUNK, and GmZF-like were isolated using DNA synthesis.

In order to obtain full-length cDNA for soybean target genes GmHD-like, GmTPP, unknown, GmRingH2 finger-like, and GmZF-like, 5′ RACE was performed using total RNA from SCN-infected soybean roots and the GeneRacer Kit (L1502-1) from Invitrogen.

The full length sequences for the soybean target genes GmHD-like, GmTPP, unknown, GmRingH2 finger-like, and GmZF-like were assembled into cDNAs corresponding to the six gene targets, designated as SEQ ID NO:4, SEQ ID NO:9, SEQ ID NO:16, SEQ ID NO:19, and SEQ ID NO:22. The full length sequences for the soybean target genes GmGLABRA-like and GmMIOX-like were determined using cDNA sequence information and are designated as SEQ ID NO:1 and SEQ ID NO:27.

Plant transformation binary vectors to express the dsRNA constructs described by SEQ ID NO:3, 6, 11, 18, 21, 24, and 29 were generated using soybean cyst nematode (SCN) inducible promoters. For this, the gene fragments described by SEQ ID NO: 3, 6, 11, 18, 21, 24, and 29 were operably linked to the SCN inducible GmMTN3 promoter (WO 2008/095887) or the At trehalose-6-phosphate phosphatase-like promoter (WO2008/071726), as designated in Table 1. The resulting plant binary vectors contain a plant transformation selectable marker consisting of a modified Arabidopsis AHAS gene conferring tolerance to the herbicide Arsenal (BASF Corporation, Florham Park, NJ).

TABLE 1

| | | | dsRNA stem | | Soybean |
|---|---|---|---|---|---|
| | | Promoter | sense | | Gene |
| Construct | | SEQ ID | fragment SEQ | | Target SEQ |
| tested | Promoter | NO: | ID NO: | Soybean Gene target | ID NO: |
| RTJ150 | AtTPP | 43 | 11 | Trehalose-6- | 9, 12, 14 |
| | | | | Phostphate | |
| | | | | Phosphatase-like | |
| RAW486 | AtTPP | 43 | 24 | Zinc Finger-like | 22, 25 |
| RAW479 | AtTPP | 43 | 21 | RingH2 finger-like | 19 |
| RAW484 | AtTPP | 43 | 6 | homeodomain-like | 4, 7 |
| RAW483 | AtTPP | 43 | 18 | unknown | 16 |
| MSB98 | AtTPP | 43 | 3 | GLABRA-like | 1 |
| RTP2615- | GmN3 | 42 | 29 | MIOX-like | 27, 30 |
| 1 | | | | | |

### Example 2

### Bioassay of dsRNA Targeted to G. Max Target Genes

The binary vectors described in Table 1 were used in the rooted plant assay system disclosed in commonly owned copending U.S. Pat. Pub. 2008/0153102. Transgenic roots were generated after transformation with the binary vectors described in Example 1. Multiple transgenic root lines were sub-cultured and inoculated with surface-decontaminated race 3 SCN second stage juveniles (J2) at the level of about 500 J2/well. Four weeks after nematode inoculation, the cyst number in each well was counted. For each transformation construct, the number of cysts per line was calculated to determine the average cyst count and standard error for the construct. The cyst count values for each transformation construct was compared to the cyst count values of an empty vector control tested in parallel to determine if the construct tested results in a reduction in cyst count. Bioassay results of constructs containing the hairpin stem sequences described by SEQ ID NOs 3, 6, 11, 18, 21, 24, and 29 resulted in a general trend of reduced soybean cyst nematode cyst count over many of the lines tested in the designated construct containing a SCN inducible promoter operably linked to each of the genes described.

### Example 3

### Identification of Additional Soybean Sequences Targeted by Binary Constructs

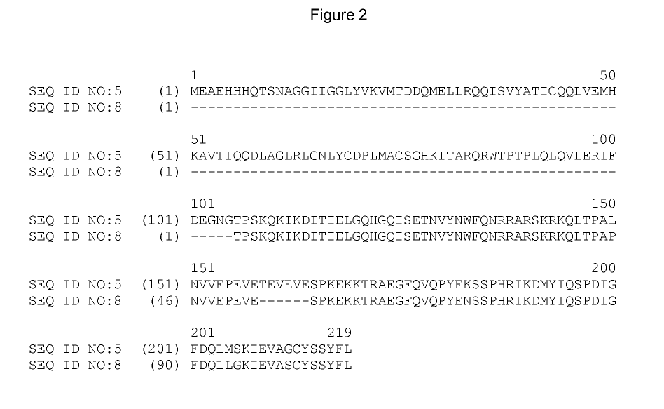

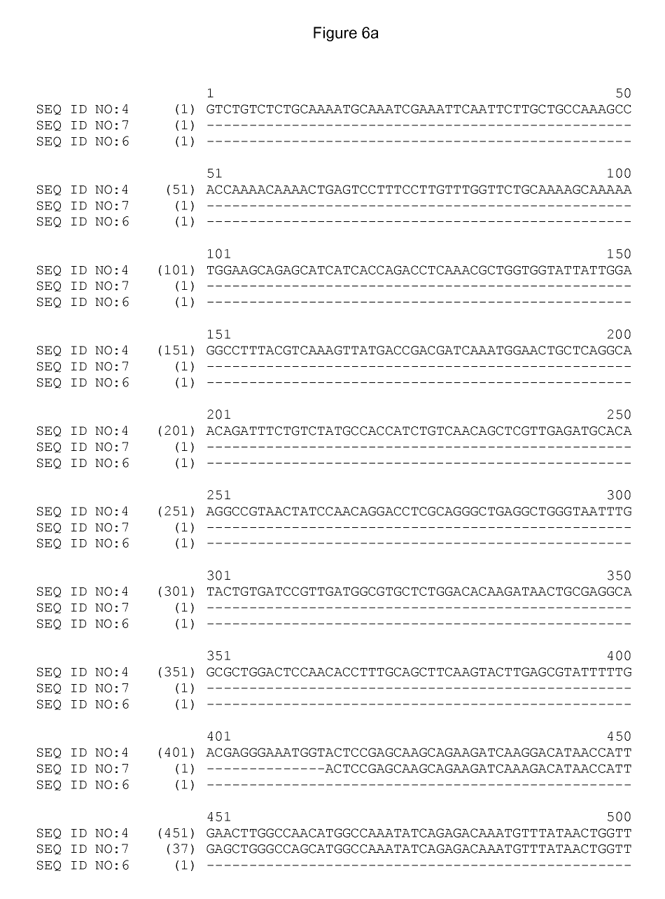

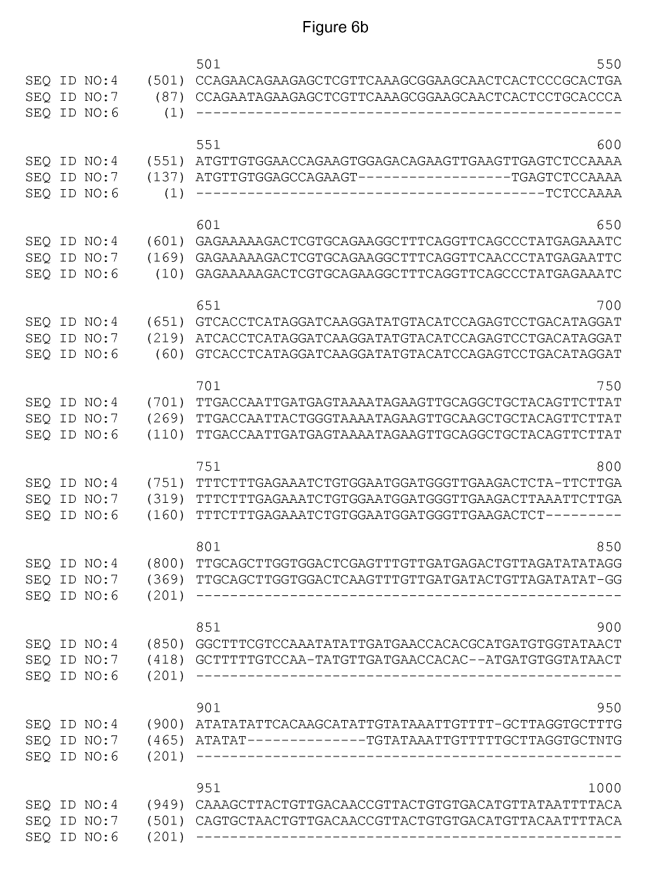

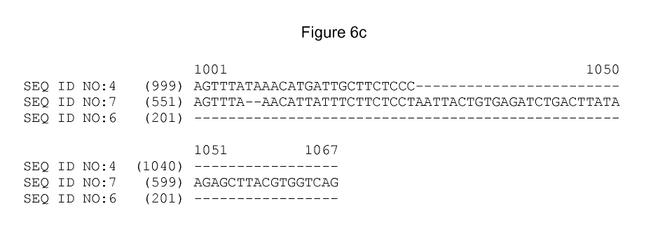

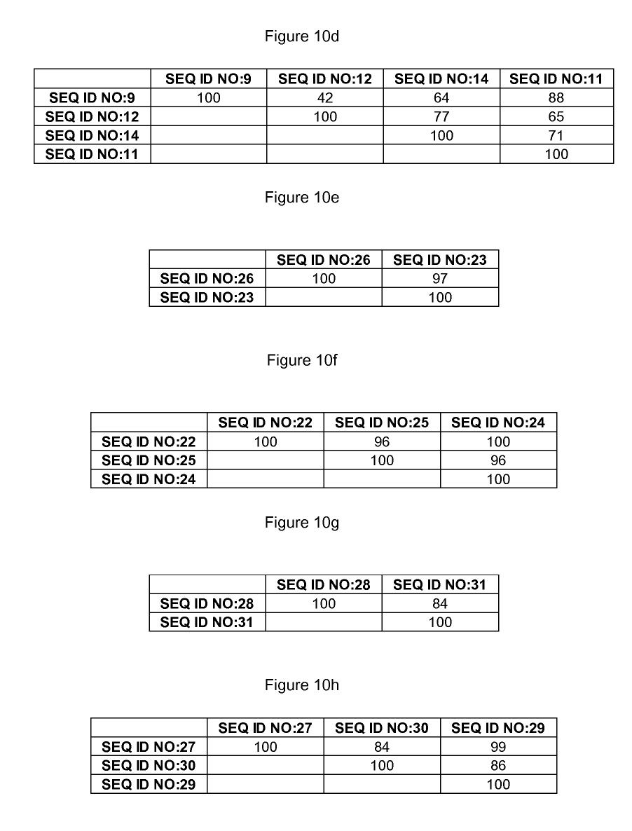

As disclosed in Example 2, the construct RAW484 results in the expression of a double stranded RNA molecule that targets SEQ ID NO:4 and results in reduced cyst count when operably linked to a SCN-inducible promoter and expressed in soybean roots. The sense fragment of the GmHD-like gene contained in RAW484, described by SEQ ID NO:6, corresponds to nucleotides 592 to 791 of the GmHD-like sequence described by SEQ ID NO:4. At least one of the resulting 21 mers derived from the processing of the double stranded RNA molecule expressed from RAW484 can target another soybean sequence described by SEQ ID NO:7. The amino acid alignment of the identified targets of the double stranded RNA molecule expressed from RAW484 described by the GmHD-like target gene SEQ ID NO:5 and GM50634465 described by SEQ ID NO:8 is shown in FIG. 2. The nucleotide alignment of the identified targets of the double stranded RNA molecule expressed from RAW484 described by the GmHD-like target gene SEQ ID NO:4, the sense fragment of the GmHD-like gene contained in RAW484 described by SEQ ID NO:6, and GM50634465 described by SEQ ID NO:7 is shown in FIG. 6. A matrix table showing the amino acid sequence percent identity of the full length amino acid sequence of the GmHD-like gene described by SEQ ID NO:5 and an additional soybean transcript target of the double stranded RNA molecule expressed by RAW484 described by SEQ ID NO:8 to each other is shown in FIG. 10a. A matrix table showing the DNA sequence percent identity of the full length transcript sequence of the GmHD-like gene described by SEQ ID NO:4, the sense fragment of the GmHD-like gene contained in RAW484 described by SEQ ID NO:6, and a additional soybean transcript target of the double stranded RNA molecule expressed by RAW484 described by SEQ ID NO:7 to each other is shown in FIG. 10b. As disclosed in Example 2, the construct RTJ150 results in the expression of a double stranded RNA molecule that targets SEQ ID NO:9 and results in reduced cyst count when operably linked to a SCN-inducible promoter and expressed in soybean roots. The sense fragment of the GmTPP-like gene contained in RTJ150, described by SEQ ID NO:11 contains exon and intron sequence of the gene corresponding to the GmTPP-like sequence described by SEQ ID NO:9. The exon regions of the sense fragment of the GmTPP-like gene contained in RTJ150, correspond to nucleotides 1 to 20 and nucleotides 144 to 552 of SEQ ID NO:11. Nucleotides 1 to 20 of SEQ ID NO:11 correspond to nucleotides 1135 to 1154 of the GmTPP-like sequence described by SEQ ID NO:9. Nucleotides 144 to 552 of SEQ ID NO:11 correspond to nucleotides 1155 to 1563 of the GmTPP-like sequence described by SEQ ID NO:9. Nucleotides 21 to 143 of SEQ ID NO:11 correspond to intron sequence of the GmTPP-like gene.

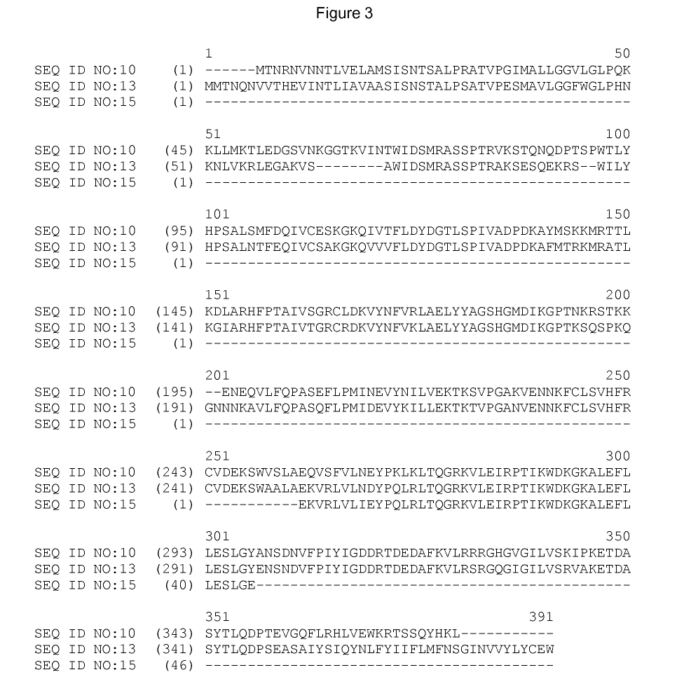

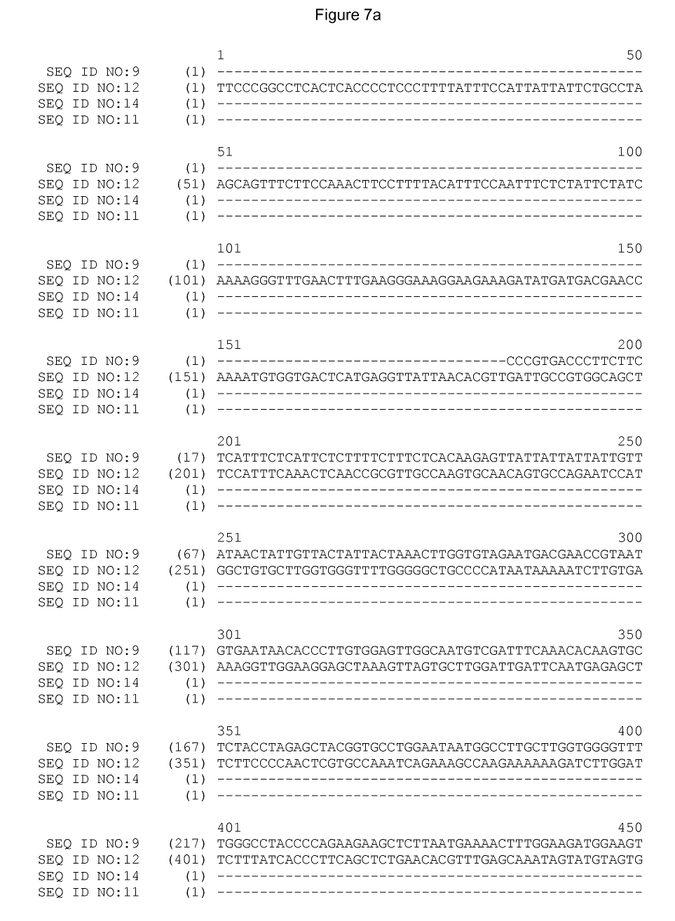

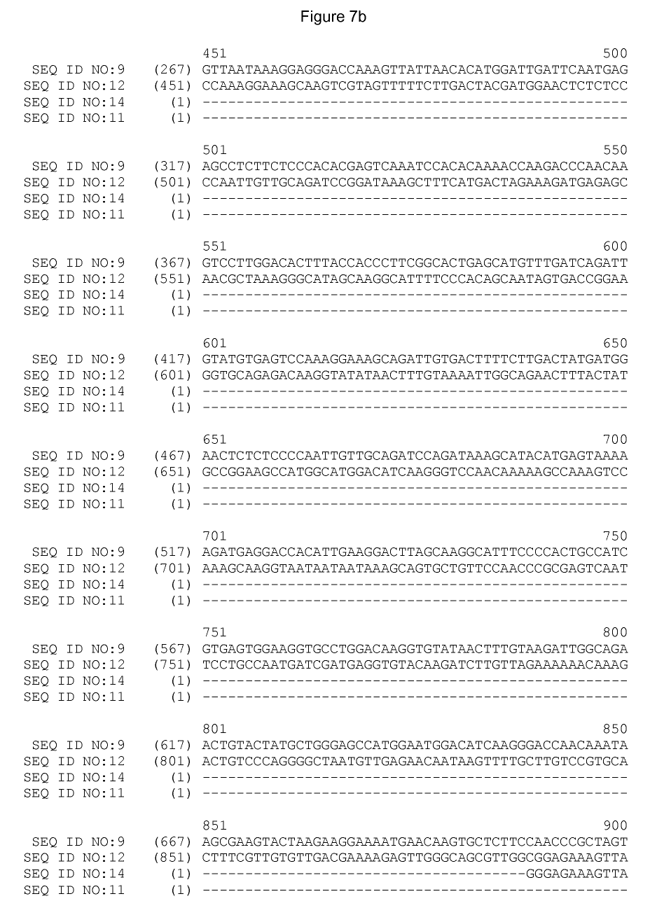

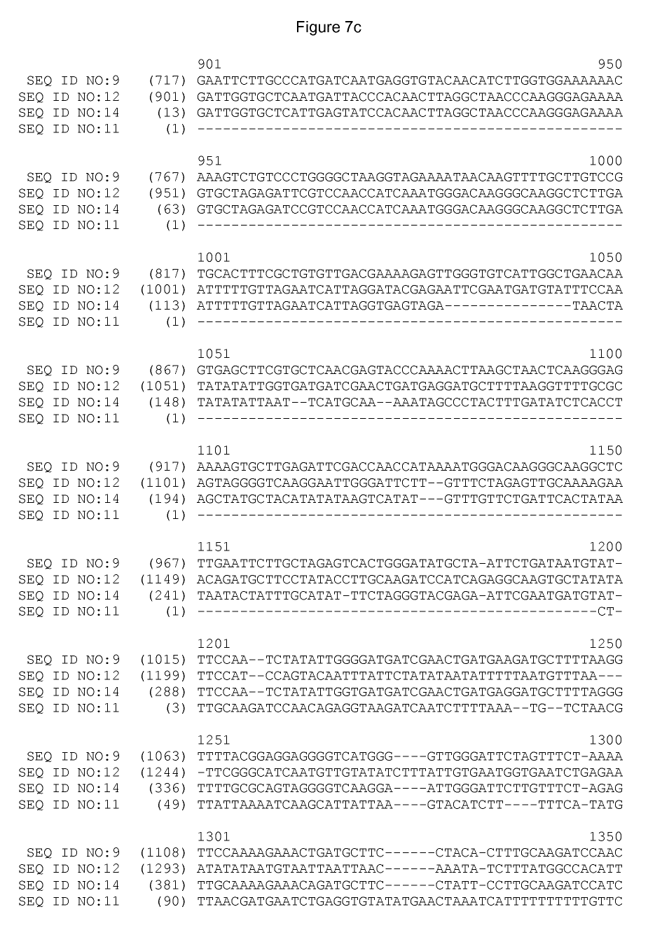

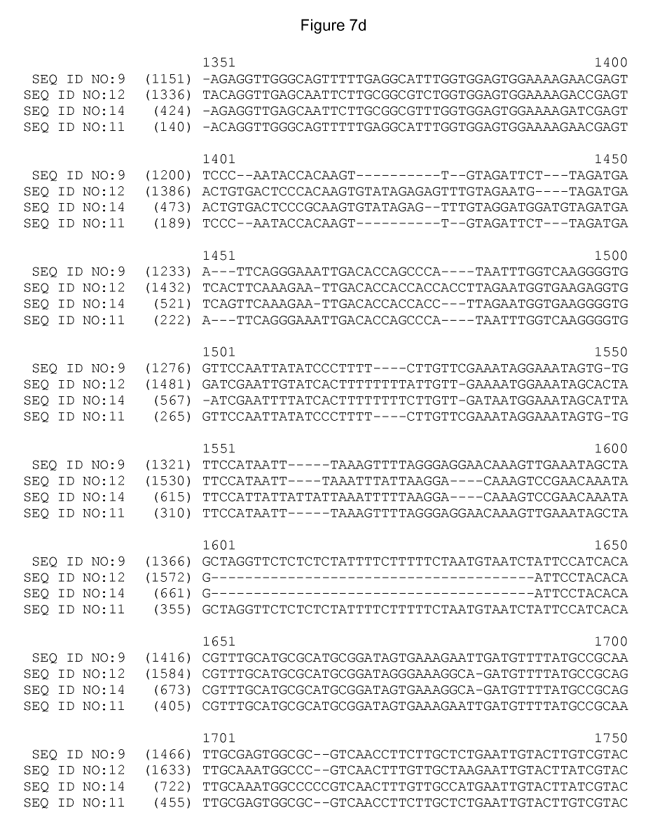

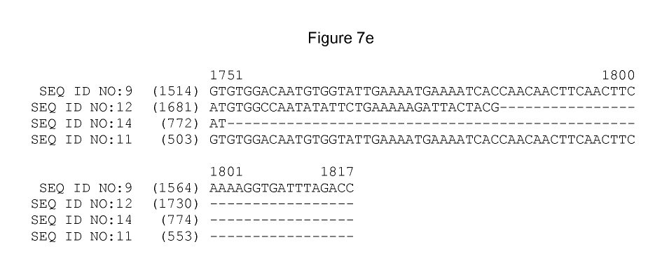

At least one of the resulting 21 mers derived from the processing of the double stranded RNA molecule expressed from RTJ150 can target other soybean sequences such as SEQ ID NO:12 and SEQ ID NO:14. The amino acid alignment of the identified targets of the double stranded RNA molecule expressed from RTJ150 described by the GmTPP-like target gene SEQ ID NO:10 and GM47125400 described by SEQ ID NO:13 and GMsq97c08 described by SEQ ID NO:15 is shown in FIG. 3. The nucleotide alignment of the identified targets of the double stranded RNA molecule expressed from RTJ150 described by the GmTPP-like target gene SEQ ID NO:9, the sense fragment of the GmTPP-like gene contained in RTJ150 described by SEQ ID NO:11, and GM47125400 described by SEQ ID NO:12 and GMsq97c08 described by SEQ ID NO:14 is shown in FIG. 7. A matrix table showing the amino acid sequence percent identity of the full length amino acid sequence of the GmTPP-like gene described by SEQ ID NO:10 and additional soybean transcript targets of the double stranded RNA molecule expressed by RTJ150 described by SEQ ID NO:13 and SEQ ID NO:15 to each other is shown in FIG. 10c. A matrix table showing the DNA sequence percent identity of the full length transcript sequence of the GmTPP-like gene described by SEQ ID NO:9, the sense fragment of the GmHD-like gene contained in RTJ150 described by SEQ ID NO:11, and additional soybean transcript targets of the double stranded RNA molecule expressed by RTJ150 described by SEQ ID NO:12 and SEQ ID NO:14 to each other is shown in FIG. 10d.

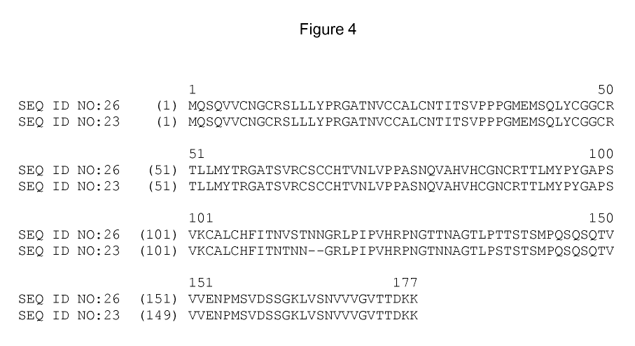

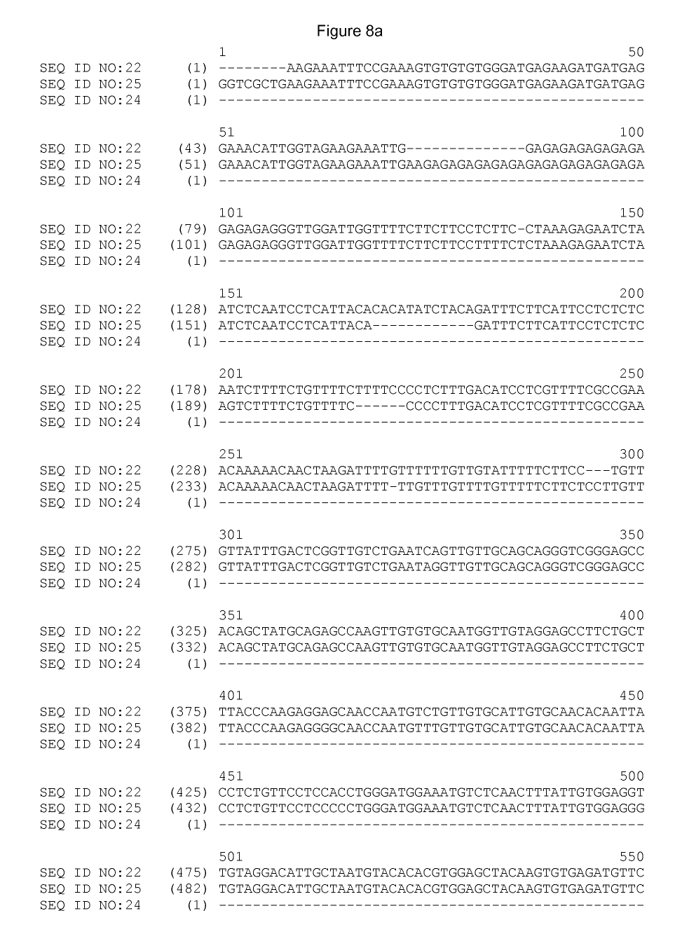

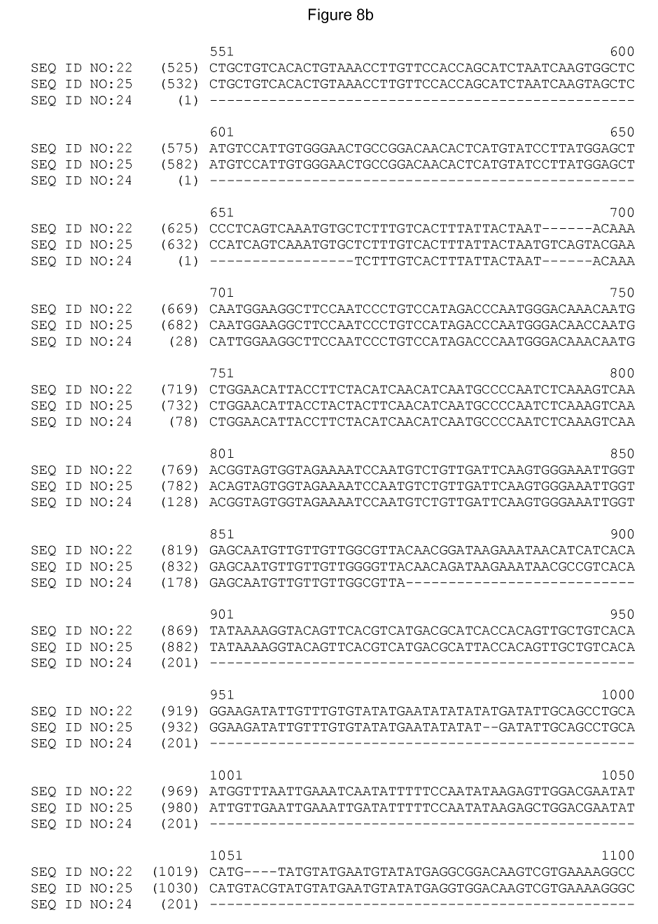

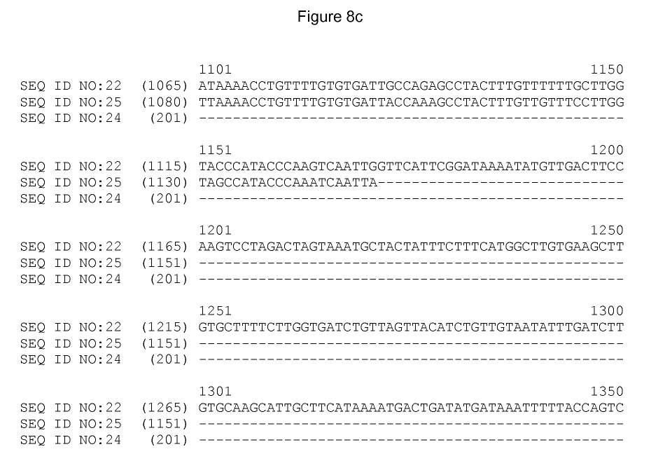

As disclosed in Example 2, the construct RAW486 results in the expression of a double stranded RNA molecule that targets SEQ ID NO:22 and results in reduced cyst count when operably linked to a SCN-inducible promoter and expressed in soybean roots. The sense fragment of the GmZF-like gene contained in RAW486, described by SEQ ID NO:24, corresponds to nucleotides 643 to 841 of the GmZF-like sequence described by SEQ ID NO:22. At least one of the resulting 21 mers derived from the processing of the double stranded RNA molecule expressed from RAW486 can target another soybean sequence described by SEQ ID NO:25. The amino acid alignment of the identified targets of the double stranded RNA molecule expressed from RAW486 described by the GmZF-like target gene SEQ ID NO:23 and the soybean gene index sequence TC248286 described by SEQ ID NO:26 is shown in FIG. 4. The nucleotide alignment of the identified targets of the double stranded RNA molecule expressed from RAW486 described by the GmZF-like target gene SEQ ID NO:22, the sense fragment of the GmHD-like gene contained in RAW486 described by SEQ ID NO:24 and the soybean gene index sequence TC248286 described by SEQ ID NO:25 is shown in FIG. 8. A matrix table showing the amino acid sequence percent identity of the full length amino acid sequence of the GmZF-like gene described by SEQ ID NO:23 and an additional soybean transcript target of the double stranded RNA molecule expressed by RAW486 described by SEQ ID NO:25 to each other is shown in FIG. 10e. A matrix table showing the DNA sequence percent identity of the full length transcript sequence of the GmZF-like gene described by SEQ ID NO:22, the sense fragment of the GmZF-like gene contained in RAW486 described by SEQ ID NO:24, and a additional soybean transcript target of the double stranded RNA molecule expressed by RAW486 described by SEQ ID NO:25 to each other is shown in FIG. 10f.

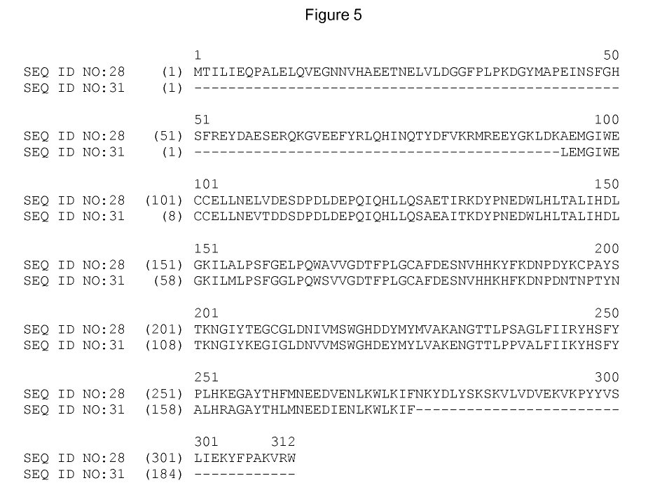

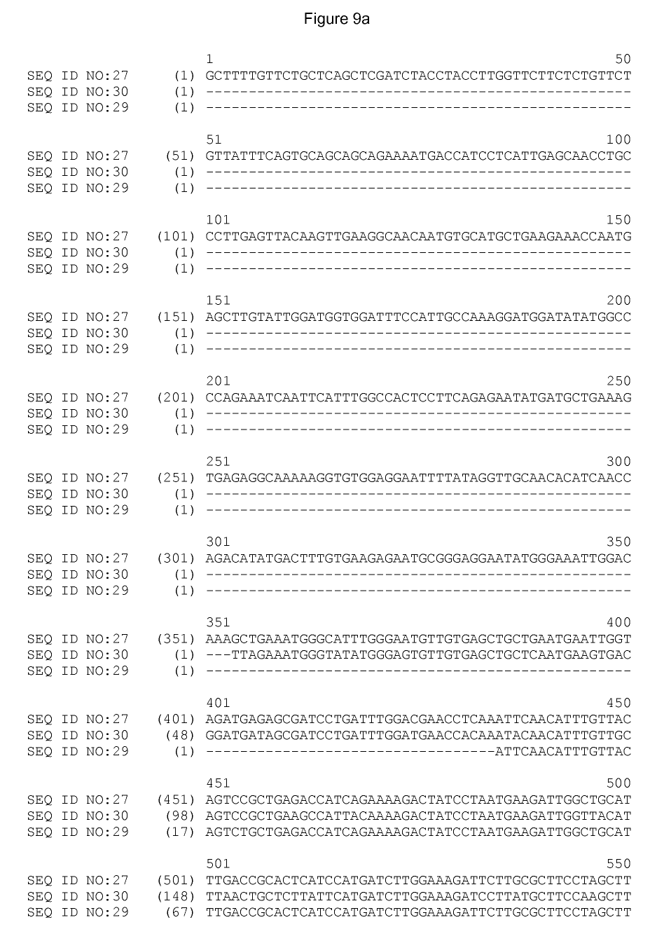

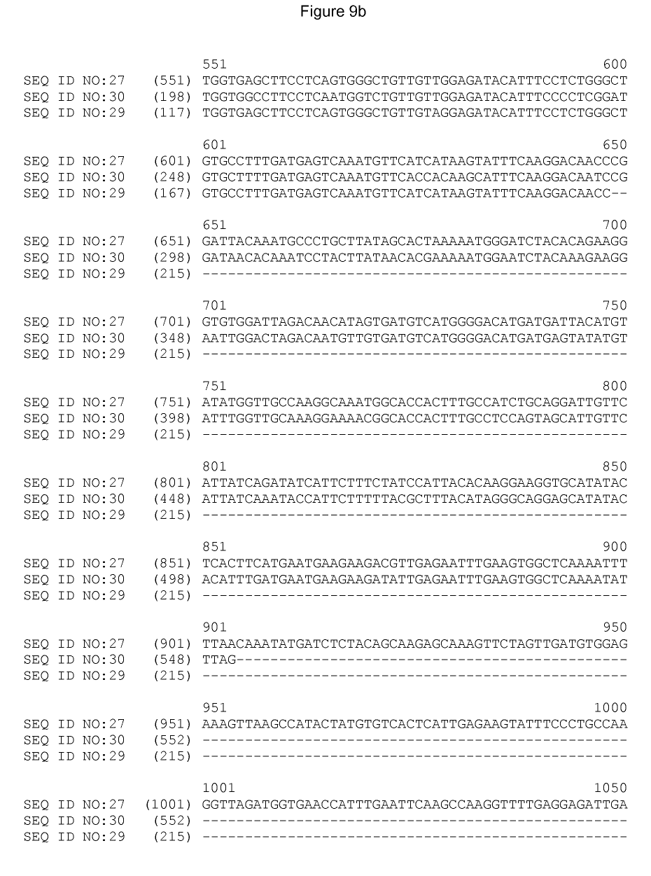

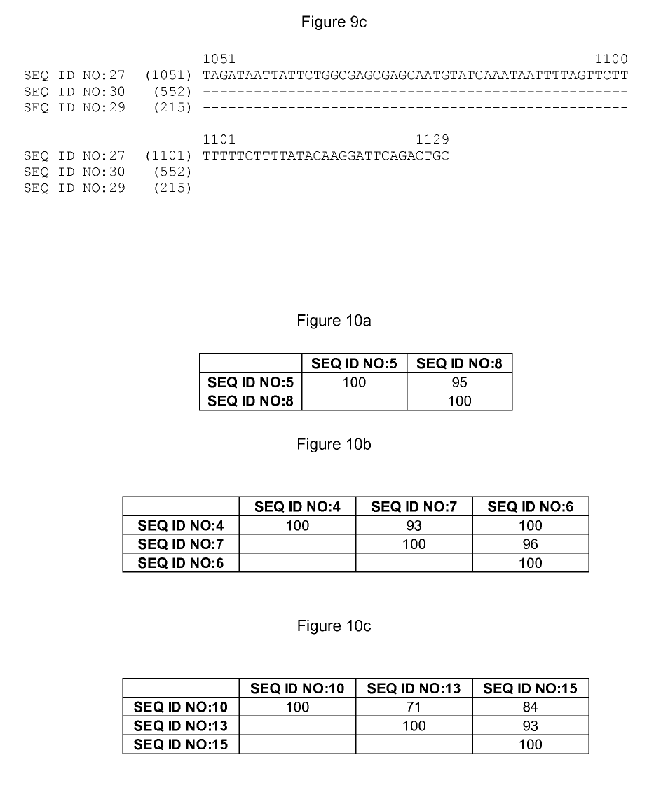

As disclosed in Example 2, the construct RTP2615-1 results in the expression of a double stranded RNA molecule that targets SEQ ID NO:27 and results in reduced cyst count when operably linked to a SCN-inducible promoter and expressed in soybean roots. The sense fragment of the GmMIOX-like gene contained in RTP2615-1, described by SEQ ID NO:29, corresponds to nucleotides 361 to 574 of the GmMIOX-like sequence described by SEQ ID NO:27. At least one of the resulting 21 mers derived from the processing of the double stranded RNA molecule expressed from RTP2615-1 can target another soybean sequence described by SEQ ID NO:30. The amino acid alignment of the identified targets of the double stranded RNA molecule expressed from RTP2615-1 described by the GmMIOX-like target gene SEQ ID NO:28 and GM50229820 described by SEQ ID NO:31 is shown in FIG. 5. The nucleotide alignment of the identified targets of the double stranded RNA molecule expressed from RTP2615-1 described by the GmMIOX-like target gene SEQ ID NO:27, the sense fragment of the GmMIOX-like gene contained in RTP2615-1 described by SEQ ID NO:29, and the hyseq sequence GM06MC04844_{—}50229820 described by SEQ ID NO:30 is shown in FIG. 9. A matrix table showing the amino acid sequence percent identity of the full length amino acid sequence of the GmMIOX-like gene described by SEQ ID NO:28 and an additional soybean transcript target of the double stranded RNA molecule expressed by RTP2615-1 described by SEQ ID NO:31 to each other is shown in FIG. 10g. A matrix table showing the DNA sequence percent identity of the full length transcript sequence of the GmMIOX-like gene described by SEQ ID NO:27, the sense fragment of the GmMIOX-like gene contained in RTP2615-1 described by SEQ ID NO:29, and a additional soybean transcript target of the double stranded RNA molecule expressed by RTP2615-1 described by SEQ ID NO:30 to each other is shown in FIG. 10h.

### Example 4

### MIOX-Like Homologs

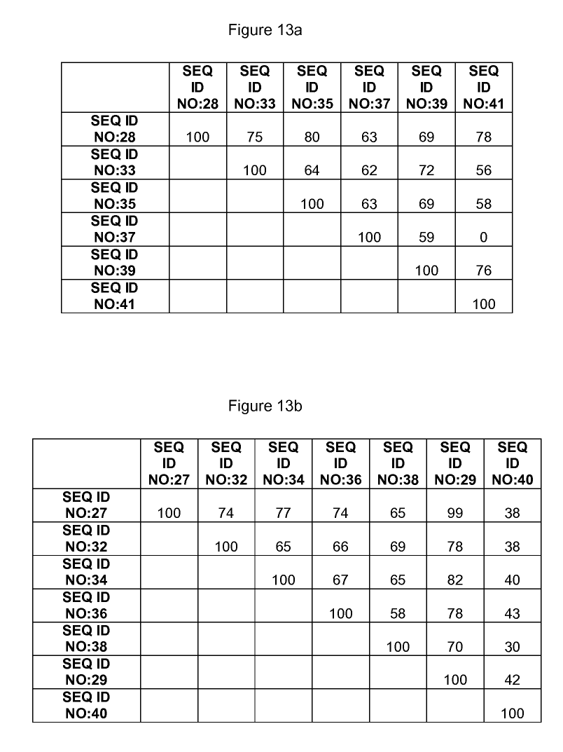

As disclosed in Example 2, the construct RTP2615-1 results in the expression of a double stranded RNA molecule that targets SEQ ID NO:27 and results in reduced cyst count when operably linked to a SCN-inducible promoter and expressed in soybean roots. As disclosed in Example 1, the putative full length transcript sequence of the gene described by SEQ ID NO:27 contains an open reading frame with the amino acid sequence disclosed as SEQ ID NO:28. The amino acid sequence described by SEQ ID NO:30 was used to identify homologous genes from other plant species subject to parasitic nematode infection. Sample genes with DNA and amino acid sequences homologous to SEQ ID NO:27 and SEQ ID NO:28, respectively, were identified and are described by SEQ ID NO:32, 34, 36, 38, and 40 and SEQ ID NO:33, 35, 37, 39, and 41. The amino acid alignment of the identified homologs to SEQ ID NO:28 is shown in FIG. 11. A matrix table showing the amino acid percent identity of the identified homologs and SEQ ID NO:28 to each other is shown in FIG. 13a. The DNA sequence alignment of the identified homologs SEQ ID NO:32, 34, 36, 38, and 40 to SEQ ID NO:27 and the sense strand contained in RTP2615-1 described by SEQ ID NO:29 is shown in FIG. 12. A matrix table showing the DNA sequence percent identity of SEQ ID NO:27, the sense strand contained in RTP2615-1 described by SEQ ID NO:29, and the identified homologs SEQ ID NO:32, 34, 36, 38, and 40 to each other is shown in FIG. 13b.

Those skilled in the art will recognize, or will be able to ascertain using no more than routine experimentation, many equivalents to the specific embodiments of the invention described herein. Such equivalents are intended to be encompassed by the following claims.

1-5. (canceled)

6. An isolated expression vector encoding a double stranded RNA comprising a first strand and a second strand complementary to the first strand, wherein the first strand is substantially identical to a portion of a plant target gene, the portion being selected from the group consisting of from about 19 to about 400 or 500 consecutive nucleotides of the target gene, wherein the double stranded RNA inhibits expression of the target gene,
and wherein the target gene is selected from the group consisting of:
(a) a polynucleotide encoding a plant GLABRA-like protein having at least 80% sequence identity to a soybean GLABRA-like protein having a sequence as set forth in SEQ ID NO:2;
(b) a polynucleotide encoding a plant homeodomain-like protein having at least 80% sequence identity to a soybean homeodomain-like protein having a sequence as set forth in SEQ ID NO:5 or SEQ ID NO:8;
(c) a polynucleotide encoding a plant trehalose-6-phosphate phosphatase-like protein;
(d) a polynucleotide encoding a plant unknown protein having at least 80% sequence identity to a soybean unknown protein having a sequence as set forth in SEQ ID NO:17;
(e) a polynucleotide encoding a RingH2 finger-like protein having at least 80% sequence identity to a soybean RingH2 finger-like protein having a sequence as set forth in SEQ ID NO:20;
(f) a polynucleotide encoding a threonine synthase-like protein;
(g) a polynucleotide encoding a zinc finger-like protein having at least 80% sequence identity to a soybean zinc finger-like protein having a sequence as set forth in SEQ ID NO:26 or SEQ ID NO:29; and
(h) a polynucleotide encoding a MIOX-like protein.

7. An isolated expression vector comprising a nucleic acid encoding a pool of double stranded RNA molecules comprising a multiplicity of RNA molecules each comprising a double stranded region having a length of about 19, 20, 21, 22, 23, or 24 nucleotides, wherein said RNA molecules are derived from a polynucleotide selected from the group consisting of:
(a) a polynucleotide encoding a plant GLABRA-like protein having at least 80% sequence identity to a soybean GLABRA-like protein having a sequence as set forth in SEQ ID NO:2;
(b) a polynucleotide encoding a plant homeodomain-like protein having at least 80% sequence identity to a soybean homeodomain-like protein having a sequence as set forth in SEQ ID NO:5 or SEQ ID NO:8;
(c) a polynucleotide encoding a plant trehalose-6-phosphate phosphatase-like protein;
(d) a polynucleotide encoding a plant unknown protein having at least 80% sequence identity to a soybean unknown protein having a sequence as set forth in SEQ ID NO:17;
(e) a polynucleotide encoding a RingH2 finger-like protein having at least 80% sequence identity to a soybean RingH2 finger-like protein having a sequence as set forth in SEQ ID NO:20;
(f) a polynucleotide encoding a threonine synthase-like protein;
(g) a polynucleotide encoding a zinc finger-like protein having at least 80% sequence identity to a soybean zinc finger-like protein having a sequence as set forth in SEQ ID NO:26 or SEQ ID NO:29; and
(h) a polynucleotide encoding a MIOX-like protein.

8. A transgenic plant capable of expressing at least one a dsRNA that is substantially identical to a portion of a plant target gene selected from the group consisting of:
(a) a polynucleotide encoding a plant GLABRA-like protein having at least 80% sequence identity to a soybean GLABRA-like protein having a sequence as set forth in SEQ ID NO:2;
(b) a polynucleotide encoding a plant homeodomain-like protein having at least 80% sequence identity to a soybean homeodomain-like protein having a sequence as set forth in SEQ ID NO:5 or SEQ ID NO:8;
(c) a polynucleotide encoding a plant trehalose-6-phosphate phosphatase-like protein;
(d) a polynucleotide encoding a plant unknown protein having at least 80% sequence identity to a soybean unknown protein having a sequence as set forth in SEQ ID NO:17;
(e) a polynucleotide encoding a RingH2 finger-like protein having at least 80% sequence identity to a soybean RingH2 finger-like protein having a sequence as set forth in SEQ ID NO:20;
(f) a polynucleotide encoding a threonine synthase-like protein;
(g) a polynucleotide encoding a zinc finger-like protein having at least 80% sequence identity to a soybean zinc finger-like protein having a sequence as set forth in SEQ ID NO:26 or SEQ ID NO:29; and
(h) a polynucleotide encoding a MIOX-like protein, wherein the dsRNA inhibits expression of the target gene in the plant root.

9. A method of making a transgenic plant capable of expressing a dsRNA comprising a first strand that is substantially identical to portion of a plant target gene and a second strand complementary to the first strand, wherein the target gene is selected from the group consisting of:
(a) a polynucleotide encoding a plant GLABRA-like protein having at least 80% sequence identity to a soybean GLABRA-like protein having a sequence as set forth in SEQ ID NO:2;
(b) a polynucleotide encoding a plant homeodomain-like protein having at least 80% sequence identity to a soybean homeodomain-like protein having a sequence as set forth in SEQ ID NO:5 or SEQ ID NO:8;
(c) a polynucleotide encoding a plant trehalose-6-phosphate phosphatase-like protein;
(d) a polynucleotide encoding a plant unknown protein having at least 80% sequence identity to a soybean unknown protein having a sequence as set forth in SEQ ID NO:17;
(e) a polynucleotide encoding a RingH2 finger-like protein having at least 80% sequence identity to a soybean RingH2 finger-like protein having a sequence as set forth in SEQ ID NO:20;
(f) a polynucleotide encoding a threonine synthase-like protein;
(g) a polynucleotide encoding a zinc finger-like protein having at least 80% sequence identity to a soybean zinc finger-like protein having a sequence as set forth in SEQ ID NO:26 or SEQ ID NO:29;
(h) a polynucleotide encoding a MIOX-like protein,
said method comprising the steps of:
(i) preparing an expression vector comprising a nucleic acid encoding the dsRNA, wherein the nucleic acid is able to form a double-stranded transcript once expressed in the plant;
(ii) transforming a recipient plant with said expression vector;
(iii) producing one or more transgenic offspring of said recipient plant; and
(iv) selecting the offspring for resistance to nematode infection.

10. A method of conferring nematode resistance to a plant, said method comprising the steps of:
(i) selecting a plant target gene selected from the group consisting of:
(a) a polynucleotide encoding a plant GLABRA-like protein having at least 80% sequence identity to a soybean GLABRA-like protein having a sequence as set forth in SEQ ID NO:2;
(b) a polynucleotide encoding a plant homeodomain-like protein having at least 80% sequence identity to a soybean homeodomain-like protein having a sequence as set forth in SEQ ID NO:5 or SEQ ID NO:8;
(c) a polynucleotide encoding a plant trehalose-6-phosphate phosphatase-like protein;
(d) a polynucleotide encoding a plant unknown protein having at least 80% sequence identity to a soybean unknown protein having a sequence as set forth in SEQ ID NO:17;
(e) a polynucleotide encoding a RingH2 finger-like protein having at least 80% sequence identity to a soybean RingH2 finger-like protein having a sequence as set forth in SEQ ID NO:20;
(f) a polynucleotide encoding a threonine synthase-like protein;
(g) a polynucleotide encoding a zinc finger-like protein having at least 80% sequence identity to a soybean zinc finger-like protein having a sequence as set forth in SEQ ID NO:26 or SEQ ID NO:29; and
(h) a polynucleotide encoding a MIOX-like protein;
(ii) preparing an expression vector comprising a nucleic acid encoding a dsRNA comprising a first strand that is substantially identical to a portion of the target gene and a second strand complementary to the first strand, wherein the nucleic acid is able to form a double-stranded transcript once expressed in the plant;
(iii) transforming a recipient plant with said nucleic acid;
(iv) producing one or more transgenic offspring of said recipient plant; and
(v) selecting the offspring for nematode resistance.


SEQUENCE LISTING (43 sequences)

SEQ ID NO: 1 - DNA, 3421 bp, Glycine max
gtccacatgttacgttttatctgtgctttcttctccaaactctctctcctcttgtctgtgcacaaatctataagcgtttcattttctacctcttttcattaaatattcatttttctttttccttctctttcacgcattagcatagtcgagcatagcggttatcatgcagggaacgagacagcacacactgaactgaaagactagggttgccatgccatcctaactcctaaggaactagtaaaagcaaagaagtatataaggcagattctgagagacccatttcatattttgtggtgctccatggcttggaaattccggggttggatgaatcaatcgacacaataatcaaggattaacagtttttcacccatgaggctgagtcattattagttgcatcaccttgtttttcttcaaaggggtattttttgagtagtagcagtttttgttgtggtgtgcttgattgaagagaggagagaaaaagaaagaaaagaaaaaataaagaagcaaaagtggctacttggatgagttttgggggatttcttgagaccaaacaaagtggtggaggtggaggtaggatcgtagcagatattccttacagcaacaacagcaacaatataatgccctctagtgctatctcgcagcctcgtttagccactcctactttggtcaaatccatgttcaactcccctggtctttctcttgcacttcaaagtgatatagatggaaaaagggatgtgaacagattaatgcccgagaatttcgagcagaatggtttgagaaggaaccgggaagaggagcatgaaagcagatctggcagtgacaacatggatggtggttctggtgatgattttgatgctgccgacaacccaccgaggaaaaaacgctatcaccgacacactcctcagcaaattcaagagcttgaatcgctcttcaaggagtgtcctcacccggatgagaaacaaaggcttgaactcagcagaaggcttaatttggaaacgaggcaagtaaagttttggttccaaaatcgaagaacacaaatgaagacacaattggaacggcacgagaactcactcctaaggcaagagaatgacaagcttagagcagaaaacatgtctatgagggaagccatgaggaatccaatatgcacaaactgtggaggtcctgcaatgattggtgaaatttcactcgaagaacagcatcttagaattgagaatgctagattgaaggacgaactagaccgtgtttgtgcactcgctggcaagtttttaggtcgacccatttcatctctaacaggctcaattgggcctccattgccaaactcaagcttggagcttggtgttgggagcaatggttttggaggattaagcactgtgccttcaacaatgcctgattttggggttggaatatcaagccctttagctatggtgtcaccttcaagtactagaccaaccacaacagcaacaacaacattggtgactcctccttctggctttgacaacagatcaattgagaggtctattgttcttgaacttgctttggctgcaatggatgagttggtgaagatggctcagactgatgagcctctttggatcagaagcttggaaggtggaagagaaattctcaaccatgacgagtacacaaggactatcactccttgcattggcttgagacccaatggctttgtcactgaggcctctagacaaactggcatggtcatcataaacagcttggcccttgttgaaacattaatggactcaaatcgttggtcagagatgttcccttgtatgattgctagaacctcaaccgctgaagttatatctaatggaataaatggaactagaaatggtgcccttcagctaatgcatgctgagcttcaagttctttctcccttggttcctgttcgtgaggtcaattttctacgcttttgcaagcagcacgcagaggggttatgggcagtggtagatgtgtccatagataccatccgagacacttctggtgcacccacttttgtgaactgtaggaggcttccttctggttgcgtggtgcaagatatgccaaatggttactctaaggtgacatgggtggaacatgcagaatacgacgaaagccaaattcaccagctctatagacccttgttgagctcaggcatggggtttggtgcacaacgttgggttgccactcttcaacgccaatgcgagtgcctagctattctaatatcctcagcagttccctctagagaacattcagcaataagttcaggtggaaggagaagcatgttgaagctggcacagcgcatgacgaacaacttctgtgctggtgtgtgtgcctcaacagtgcacaagtggaacaagctgaacgcgggaaacgtgggggaggacgtgagggtgatgacgaggaagagcgtggatgaccccggtgaaccgccggggatcgtcctcagtgccgccacctcggtgtggcttcccgtctcgccacagaggctcttcgacttcctccgtgacgagcggctccggagtgagtgggacatcctctccaacggtggaccaatgcaagagatggctcacattgccaagggacaagaccatgctaactgtgtctccctccttagagccagtgctataaatgcgaaccagagcagcatgttgattctgcaagagacgtgcacagacgcgtcggggtcgcttgtggtgtacgcgccggtggacattccggcaatgcacgtcgtgatgaacggcggcgactctgcttacgtggcgcttcttccgtcggggttcgccatcgtgcccgacgggtccgtcgaggagaacggtggcgcgtcgcagcagagggcggcgagtggcgggtgcctcctgacggtggcgtttcagattctggtgaacagcctccccacggcgaagctcacggtggagtcggtggagacggtgaacaacctcatctcctgcaccgtgcagaagatcaaatcagcgcttcactgcgaaagctgaaagtcacgtgactctggctttgtctattattattattattatttttgttttggagaatttaggttacattttgtagttaaaggtggcgcgtgtgaaggacgcgaggggtgggggtgttgagtcaagaacgaaccgcgcgtgggtgagagaactccttgcatggtgatatgaggatccgagaccttttccgggttgggttaggattttcaacacaaagcaagggtggtggttcgggtattgactcgggtcgacccctttgtttgctccatacagtagcagaagaaggaaaaaaaaaatgaatagaaatgtgagaaacaaaaaggaacaaaattgtgtttctggttaggaatatttattgtaagttatcatcattttgatttgtattgacttggtgggagtatgctagaattggatctagtagtttgttgcatatgatc

SEQ ID NO: 2 - protein, 820 aa, Glycine max
MSFGGFLETKQSGGGGGRIVADIPYSNNSNNIMPSSAISQPRLATPTLVKSMFNSPGLSLALQSDIDGKRDVNRLMPENFEQNGLRRNREEEHESRSGSDNMDGGSGDDFDAADNPPRKKRYHRHTPQQIQELESLFKECPHPDEKQRLELSRRLNLETRQVKFWFQNRRTQMKTQLERHENSLLRQENDKLRAENMSMREAMRNPICTNCGGPAMIGEISLEEQHLRIENARLKDELDRVCALAGKFLGRPISSLTGSIGPPLPNSSLELGVGSNGFGGLSTVPSTMPDFGVGISSPLAMVSPSSTRPTTTATTTLVTPPSGFDNRSIERSIVLELALAAMDELVKMAQTDEPLWIRSLEGGREILNHDEYTRTITPCIGLRPNGFVTEASRQTGMVIINSLALVETLMDSNRWSEMFPCMIARTSTAEVISNGINGTRNGALQLMHAELQVLSPLVPVREVNFLRFCKQHAEGLWAVVDVSIDTIRDTSGAPTFVNCRRLPSGCVVQDMPNGYSKVTWVEHAEYDESQIHQLYRPLLSSGMGFGAQRWVATLQRQCECLAILISSAVPSREHSAISSGGRRSMLKLAQRMTNNFCAGVCASTVHKWNKLNAGNVGEDVRVMTRKSVDDPGEPPGIVLSAATSVWLPVSPQRLFDFLRDERLRSEWDILSNGGPMQEMAHIAKGQDHANCVSLLRASAINANQSSMLILQETCTDASGSLVVYAPVDIPAMHVVMNGGDSAYVALLPSGFAIVPDGSVEENGGASQQRAASGGCLLTVAFQILVNSLPTAKLTVESVETVNNLISCTVQKIKSALHCES

SEQ ID NO: 3 - DNA, 360 bp, Glycine max
ggtgcacaacgttgggttgccactcttcaacgccaatgcgagtgcctagctattctaatatcctcagcagttccctctagagaacattcagcaataagttcaggtggaaggagaagcatgttgaagctggcacagcgcatgacgaacaacttctgtgctggtgtgtgtgcctcaacagtgcacaagtggaacaagctgaacgcgggaaacgtgggggaggacgtgagggtgatgacgaggaagagcgtggatgaccccggtgaaccgccggggatcgtcctcagtgccgccacctcggtgtggcttcccgtctcgccgcagaggctcttcgacttcctccgtgacgagcggctccggagt

SEQ ID NO: 4 - DNA, 1024 bp, Glycine max
gtctgtctctgcaaaatgcaaatcgaaattcaattcttgctgccaaagccaccaaaacaaaactgagtcctttccttgtttggttctgcaaaagcaaaaatggaagcagagcatcatcaccagacctcaaacgctggtggtattattggaggcctttacgtcaaagttatgaccgacgatcaaatggaactgctcaggcaacagatttctgtctatgccaccatctgtcaacagctcgttgagatgcacaaggccgtaactatccaacaggacctcgcagggctgaggctgggtaatttgtactgtgatccgttgatggcgtgctctggacacaagataactgcgaggcagcgctggactccaacacctttgcagcttcaagtacttgagcgtatttttgacgagggaaatggtactccgagcaagcagaagatcaaggacataaccattgaacttggccaacatggccaaatatcagagacaaatgtttataactggttccagaacagaagagctcgttcaaagcggaagcaactcactcccgcactgaatgttgtggaaccagaagtggagacagaagttgaagttgagtctccaaaagagaaaaagactcgtgcagaaggctttcaggttcagccctatgagaaatcgtcacctcataggatcaaggatatgtacatccagagtcctgacataggatttgaccaattgatgagtaaaatagaagttgcaggctgctacagttcttattttctttgagaaatctgtggaatggatgggttgaagactctattcttgattgcagcttggtggactcgagtttgttgatgagactgttagatatataggggctttcgtccaaatatattgatgaaccacacgcatgatgtggtataactatatatattcacaagcatattgtataaattgttttgcttaggtgctttgcaaagcttactgttgacaaccgttactgtgtgacatgttataattttacaagtttataaacatgattgcttctccc

SEQ ID NO: 5 - protein, 219 aa, Glycine max
MEAEHHHQTSNAGGIIGGLYVKVMTDDQMELLRQQISVYATICQQLVEMHKAVTIQQDLAGLRLGNLYCDPLMACSGHKITARQRWTPTPLQLQVLERIFDEGNGTPSKQKIKDITIELGQHGQISETNVYNWFQNRRARSKRKQLTPALNVVEPEVETEVEVESPKEKKTRAEGFQVQPYEKSSPHRIKDMYIQSPDIGFDQLMSKIEVAGCYSSYFL

SEQ ID NO: 6 - DNA, 200 bp, Glycine max
tctccaaaagagaaaaagactcgtgcagaaggctttcaggttcagccctatgagaaatcgtcacctcataggatcaaggatatgtacatccagagtcctgacataggatttgaccaattgatgagtaaaatagaagttgcaggctgctacagttcttattttctttgagaaatctgtggaatggatgggttgaagactct

SEQ ID NO: 7 - DNA, 615 bp, Glycine max
  misc_feature (498)..(498): n is a, c, g, or t
actccgagcaagcagaagatcaaagacataaccattgagctgggccagcatggccaaatatcagagacaaatgtttataactggttccagaatagaagagctcgttcaaagcggaagcaactcactcctgcacccaatgttgtggagccagaagttgagtctccaaaagagaaaaagactcgtgcagaaggctttcaggttcaaccctatgagaattcatcacctcataggatcaaggatatgtacatccagagtcctgacataggatttgaccaattactgggtaaaatagaagttgcaagctgctacagttcttattttctttgagaaatctgtggaatggatgggttgaagacttaaattcttgattgcagcttggtggactcaagtttgttgatgatactgttagatatatgggctttttgtccaatatgttgatgaaccacacatgatgtggtataactatatattgtataaattgtttttgcttaggtgctntgcagtgctaactgttgacaaccgttactgtgtgacatgttacaattttacaagtttaaacattatttcttctcctaattactgtgagatctgacttataagagcttacgtggtcag

SEQ ID NO: 8 - protein, 108 aa, Glycine max
TPSKQKIKDITIELGQHGQISETNVYNWFQNRRARSKRKQLTPAPNVVEPEVESPKEKKTRAEGFQVQPYENSSPHRIKDMYIQSPDIGFDQLLGKIEVASCYSSYFL

SEQ ID NO: 9 - DNA, 1580 bp, Glycine max
cccgtgacccttcttctcatttctcattctcttttctttctcacaagagttattattattattgttataactattgttactattactaaacttggtgtagaatgacgaaccgtaatgtgaataacacccttgtggagttggcaatgtcgatttcaaacacaagtgctctacctagagctacggtgcctggaataatggccttgcttggtggggttttgggcctaccccagaagaagctcttaatgaaaactttggaagatggaagtgttaataaaggagggaccaaagttattaacacatggattgattcaatgagagcctcttctcccacacgagtcaaatccacacaaaaccaagacccaacaagtccttggacactttaccacccttcggcactgagcatgtttgatcagattgtatgtgagtccaaaggaaagcagattgtgacttttcttgactatgatggaactctctccccaattgttgcagatccagataaagcatacatgagtaaaaagatgaggaccacattgaaggacttagcaaggcatttccccactgccatcgtgagtggaaggtgcctggacaaggtgtataactttgtaagattggcagaactgtactatgctgggagccatggaatggacatcaagggaccaacaaataagcgaagtactaagaaggaaaatgaacaagtgctcttccaacccgctagtgaattcttgcccatgatcaatgaggtgtacaacatcttggtggaaaaaacaaagtctgtccctggggctaaggtagaaaataacaagttttgcttgtccgtgcactttcgctgtgttgacgaaaagagttgggtgtcattggctgaacaagtgagcttcgtgctcaacgagtacccaaaacttaagctaactcaagggagaaaagtgcttgagattcgaccaaccataaaatgggacaagggcaaggctcttgaattcttgctagagtcactgggatatgctaattctgataatgtatttccaatctatattggggatgatcgaactgatgaagatgcttttaaggttttacggaggaggggtcatggggttgggattctagtttctaaaattccaaaagaaactgatgcttcctacactttgcaagatccaacagaggttgggcagtttttgaggcatttggtggagtggaaaagaacgagttcccaataccacaagttgtagattcttagatgaattcagggaaattgacaccagcccataatttggtcaaggggtggttccaattatatcccttttcttgttcgaaataggaaatagtgtgttccataatttaaagttttagggaggaacaaagttgaaatagctagctaggttctctctctattttctttttctaatgtaatctattccatcacacgtttgcatgcgcatgcggatagtgaaagaattgatgttttatgccgcaattgcgagtggcgcgtcaaccttcttgctctgaattgtacttgtcgtacgtgtggacaatgtggtattgaaaatgaaaatcaccaacaacttcaacttcaaaaggtgatttagacc

SEQ ID NO: 10 - protein, 372 aa, Glycine max
MTNRNVNNTLVELAMSISNTSALPRATVPGIMALLGGVLGLPQKKLLMKTLEDGSVNKGGTKVINTWIDSMRASSPTRVKSTQNQDPTSPWTLYHPSALSMFDQIVCESKGKQIVTFLDYDGTLSPIVADPDKAYMSKKMRTTLKDLARHFPTAIVSGRCLDKVYNFVRLAELYYAGSHGMDIKGPTNKRSTKKENEQVLFQPASEFLPMINEVYNILVEKTKSVPGAKVENNKFCLSVHFRCVDEKSWVSLAEQVSFVLNEYPKLKLTQGRKVLEIRPTIKWDKGKALEFLLESLGYANSDNVFPIYIGDDRTDEDAFKVLRRRGHGVGILVSKIPKETDASYTLQDPTEVGQFLRHLVEWKRTSSQYHKL

SEQ ID NO: 11 - DNA, 552 bp, Glycine max
ctttgcaagatccaacagaggtaagatcaatcttttaaatgtctaacgttattaaaatcaagcattattaagtacatctttttcatatgttaacgatgaatctgaggtgtatatgaactaaatcattttttttttgttcacaggttgggcagtttttgaggcatttggtggagtggaaaagaacgagttcccaataccacaagttgtagattcttagatgaattcagggaaattgacaccagcccataatttggtcaaggggtggttccaattatatcccttttcttgttcgaaataggaaatagtgtgttccataatttaaagttttagggaggaacaaagttgaaatagctagctaggttctctctctattttctttttctaatgtaatctattccatcacacgtttgcatgcgcatgcggatagtgaaagaattgatgttttatgccgcaattgcgagtggcgcgtcaaccttcttgctctgaattgtacttgtcgtacgtgtggacaatgtggtattgaaaatgaaaatcaccaacaacttcaacttc

SEQ ID NO: 12 - DNA, 1714 bp, Glycine max
ttcccggcctcactcacccctcccttttatttccattattattctgcctaagcagtttcttccaaacttccttttacatttccaatttctctattctatcaaaagggtttgaactttgaagggaaaggaagaaagatatgatgacgaaccaaaatgtggtgactcatgaggttattaacacgttgattgccgtggcagcttccatttcaaactcaaccgcgttgccaagtgcaacagtgccagaatccatggctgtgcttggtgggttttgggggctgccccataataaaaatcttgtgaaaaggttggaaggagctaaagttagtgcttggattgattcaatgagagcttcttccccaactcgtgccaaatcagaaagccaagaaaaaagatcttggattctttatcacccttcagctctgaacacgtttgagcaaatagtatgtagtgccaaaggaaagcaagtcgtagtttttcttgactacgatggaactctctccccaattgttgcagatccggataaagctttcatgactagaaagatgagagcaacgctaaagggcatagcaaggcattttcccacagcaatagtgaccggaaggtgcagagacaaggtatataactttgtaaaattggcagaactttactatgccggaagccatggcatggacatcaagggtccaacaaaaagccaaagtccaaagcaaggtaataataataaagcagtgctgttccaacccgcgagtcaattcctgccaatgatcgatgaggtgtacaagatcttgttagaaaaaacaaagactgtcccaggggctaatgttgagaacaataagttttgcttgtccgtgcactttcgttgtgttgacgaaaagagttgggcagcgttggcggagaaagttagattggtgctcaatgattacccacaacttaggctaacccaagggagaaaagtgctagagattcgtccaaccatcaaatgggacaagggcaaggctcttgaatttttgttagaatcattaggatacgagaattcgaatgatgtatttccaatatatattggtgatgatcgaactgatgaggatgcttttaaggttttgcgcagtaggggtcaaggaattgggattcttgtttctagagttgcaaaagaaacagatgcttcctataccttgcaagatccatcagaggcaagtgctatatattccatccagtacaatttattctatataatatttttaatgtttaattcgggcatcaatgttgtatatctttattgtgaatggtgaatctgagaaatatataatgtaattaattaacaaatatctttatggccacatttacaggttgagcaattcttgcggcgtctggtggagtggaaaagaccgagtactgtgactcccacaagtgtatagagagtttgtagaatgtagatgatcacttcaaagaattgacaccaccaccaccttagaatggtgaagaggtggatcgaattgtatcacttttttttattgttgaaaatggaaatagcactattccataatttaaatttattaaggacaaagtccgaacaaatagattcctacacacgtttgcatgcgcatgcggatagggaaaggcagatgttttatgccgcagttgcaaatggcccgtcaactttgttgctaagaattgtacttatcgtacatgtggccaatatattctgaaaaagattactacg

SEQ ID NO: 13 - protein, 381 aa, Glycine max
MMTNQNVVTHEVINTLIAVAASISNSTALPSATVPESMAVLGGFWGLPHNKNLVKRLEGAKVSAWIDSMRASSPTRAKSESQEKRSWILYHPSALNTFEQIVCSAKGKQVVVFLDYDGTLSPIVADPDKAFMTRKMRATLKGIARHFPTAIVTGRCRDKVYNFVKLAELYYAGSHGMDIKGPTKSQSPKQGNNNKAVLFQPASQFLPMIDEVYKILLEKTKTVPGANVENNKFCLSVHFRCVDEKSWAALAEKVRLVLNDYPQLRLTQGRKVLEIRPTIKWDKGKALEFLLESLGYENSNDVFPIYIGDDRTDEDAFKVLRSRGQGIGILVSRVAKETDASYTLQDPSEASAIYSIQYNLFYIIFLMFNSGINVVYLYCEW

SEQ ID NO: 14 - DNA, 773 bp, Glycine max
gggagaaagttagattggtgctcattgagtatccacaacttaggctaacccaagggagaaaagtgctagagatccgtccaaccatcaaatgggacaagggcaaggctcttgaatttttgttagaatcattaggtgagtagataactatatatattaattcatgcaaaaatagccctactttgatatctcacctagctatgctacatatataagtcatatgtttgttctgattcactataataatactatttgcatatttctagggtacgagaattcgaatgatgtatttccaatctatattggtgatgatcgaactgatgaggatgcttttagggttttgcgcagtaggggtcaaggaattgggattcttgtttctagagttgcaaaagaaacagatgcttcctattccttgcaagatccatcagaggttgagcaattcttgcggcgtttggtggagtggaaaagatcgagtactgtgactcccgcaagtgtatagagtttgtaggatggatgtagatgatcagttcaaagaattgacaccaccaccttagaatggtgaaggggtgatcgaattttatcacttttttttcttgttgataatggaaatagcattattccattattattattaaatttttaaggacaaagtccgaacaaatagattcctacacacgtttgcatgcgcatgcggatagtgaaaggcagatgttttatgccgcagttgcaaatggcccccgtcaactttgttgccatgaattgtacttatcgtacat

SEQ ID NO: 15 - protein, 45 aa, Glycine max
EKVRLVLIEYPQLRLTQGRKVLEIRPTIKWDKGKALEFLLESLGE

SEQ ID NO: 16 - DNA, 1185 bp, Glycine max
tctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcagaattaaccctcactaaagggactagtcctgcaggtttaaacgaattcgcccttggacactgacatggactgaaggagtagaaaatccttccttcccaaccatgaacaagaaaccctcaagaaacctcctcacccccaaaaccaaaccctttttattaaccttaaccacgaagtaaaacacaaacacaacctccatgtgcaccttaaccacaacaaacacaattcgtgtcttattacttattttcctctaaatctaacccaactcaacccgtgctcatccctaattatgggaaactgcgtgttcaaaggtttacaccacggcgtttctgaaaacatgatggtgaaagtggttacctcaaacggaggcatcatggaactcttctctcccataaccgtggagtgcataaccagcgagttccccggccacggcatcttccgaagccgccgcgacatgttctccgaaccgctccccaaaaacgaagagctccgcggcggagaagtctactacctcctccctctaaacccttcttcttctcgcaagagcttgacgagacaattctccgacgccgaggccaccttaacaccgtaccgaatgtcaacgtgcgagaaaaataacaacaacaacaacgtgtactcggaaccacccgaggtgattccgagatacaatagtagtggggtgtggaaggtgaagttggtgataagccccgagaagctgtcggagattttgtcgcaggagtcaaggacggaggcgttgatagagagcgtgaggacggtggcgaagtgtggtaacggcgtgccgtcgtcggtggcgaactccgatcagtggagtgtggcaagcagttggaaaggttctatgtcggagaagatgggtttacaatagctagctatatgttaattaattgatttttttcctacttttttgacttttttttgctagtgtttaacgaccctgattattattttcttc

SEQ ID NO: 17 - protein, 197 aa, Glycine max
MGNCVFKGLHHGVSENMMVKVVTSNGGIMELFSPITVECITSEFPGHGIFRSRRDMFSEPLPKNEELRGGEVYYLLPLNPSSSRKSLTRQFSDAEATLTPYRMSTCEKNNNNNNVYSEPPEVIPRYNSSGVWKVKLVISPEKLSEILSQESRTEALIESVRTVAKCGNGVPSSVANSDQWSVASSWKGSMSEKMGLQ

SEQ ID NO: 18 - DNA, 200 bp, Glycine max
gtgataagccccgagaagctgtcggagattttgtcgcaggagtcaaggacggaggcgttgatagagagcgtgaggacggtggcgaagtgtggtaacggcgtgccgtcgtcggtggcgaactccgatcagtggagtgtggcaagcagttggaaaggttctatgtcggagaagatgggtttacaatagctagctatatgtta

SEQ ID NO: 19 - DNA, 1033 bp, Glycine max
cttctcgtgttcttgtagacattgctgttgttatatttatatatataactcttccttaaacccgaatctgtgacattgaacaaacggcactggaatttctgagataaccacatggagtttgtgccaaatcaatgccctttgatgggttcctttgggaatttcgttgagagggtcaaaagggttggtaccctcttcgtctctgccatcattgggaacatattctctgcgatcttgaccttctgctttgcgttagttggcactttgttgggtgctatgactggtgccttgataggccaagagacagagagtggtttcattcgaggggctgctataggtgccatatcaggagctgttttttccattgaagtttttgaatcttcccttgttctttggaaatctgacgaatctggaattgggtgtgtcttatacttgattgatgttcttggtagcctattgagtggaagactagtgcgtgaaaggataggtccagccatgttgagtgctgtccaaagtcagatgggtgctgttgaaataagctttgatgaggtacaaaacctctttgacattggtggcgccaaaggtttatcgagagattcagttgaaaagatcccaaagatcacaattactagtgacaacaatgttgatgcttctggggagaaagattcatgttcagtttgccttcaggactttcagcttggggagactggtagaagtttgccccattgtcatcacatatttcacctaccttgcattgatatgtggctgatgaaacatggttcctgcccattatgcagaagggatctgggtaattttgtaaatgcaaagtacaaaccgtaaaaatagctaggttctttcatttttatttttttttatataagggaatttacttttaggaatgtatagtatggttaaatgtagtaggaactaggaaccagccatgtcactcatgagtgtcataattgtaataagttactacaaagaaaattttgacataaatcagctgccatttcttgtataaacaaatctttcgttt

SEQ ID NO: 20 - protein, 241 aa, Glycine max
MEFVPNQCPLMGSFGNFVERVKRVGTLFVSAIIGNIFSAILTFCFALVGTLLGAMTGALIGQETESGFIRGAAIGAISGAVFSIEVFESSLVLWKSDESGIGCVLYLIDVLGSLLSGRLVRERIGPAMLSAVQSQMGAVEISFDEVQNLFDIGGAKGLSRDSVEKIPKITITSDNNVDASGEKDSCSVCLQDFQLGETGRSLPHCHHIFHLPCIDMWLMKHGSCPLCRRDLGNFVNAKYKP

SEQ ID NO: 21 - DNA, 200 bp, Glycine max
ggtacaaaacctctttgacattggtggcgccaaaggtttatcgagagattcagttgaaaagatcccaaagatcacaattactagtgacaacaatgttgatgcttctggggagaaagattcatgttcagtttgccttcaggactttcagcttggggagactggtagaagtttgccccattgtcatcacatatttcacctac

SEQ ID NO: 22 - DNA, 1314 bp, Glycine max
aagaaatttccgaaagtgtgtgtgggatgagaagatgatgaggaaacattggtagaagaaattggagagagagagagagagagagggttggattggttttcttcttcctcttcctaaagagaatctaatctcaatcctcattacacacatatctacagatttcttcattcctctctcaatcttttctgttttcttttcccctctttgacatcctcgttttcgccgaaacaaaaacaactaagattttgttttttgttgtatttttcttcctgttgttatttgactcggttgtctgaatcagttgttgcagcagggtcgggagccacagctatgcagagccaagttgtgtgcaatggttgtaggagccttctgctttacccaagaggagcaaccaatgtctgttgtgcattgtgcaacacaattacctctgttcctccacctgggatggaaatgtctcaactttattgtggaggttgtaggacattgctaatgtacacacgtggagctacaagtgtgagatgttcctgctgtcacactgtaaaccttgttccaccagcatctaatcaagtggctcatgtccattgtgggaactgccggacaacactcatgtatccttatggagctccctcagtcaaatgtgctctttgtcactttattactaatacaaacaatggaaggcttccaatccctgtccatagacccaatgggacaaacaatgctggaacattaccttctacatcaacatcaatgccccaatctcaaagtcaaacggtagtggtagaaaatccaatgtctgttgattcaagtgggaaattggtgagcaatgttgttgttggcgttacaacggataagaaataacatcatcacatataaaaggtacagttcacgtcatgacgcatcaccacagttgctgtcacaggaagatattgtttgtgtatatgaatatatatatgatattgcagcctgcaatggtttaattgaaatcaatatttttccaatataagagttggacgaatatcatgtatgtatgaatgtatatgaggcggacaagtcgtgaaaaggccataaaacctgttttgtgtgattgccagagcctactttgttttttgcttggtacccatacccaagtcaattggttcattcggataaaatatgttgacttccaagtcctagactagtaaatgctactatttctttcatggcttgtgaagcttgtgcttttcttggtgatctgttagttacatctgttgtaatatttgatcttgtgcaagcattgcttcataaaatgactgatatgataaatttttaccagtc

SEQ ID NO: 23 - protein, 175 aa, Glycine max
MQSQVVCNGCRSLLLYPRGATNVCCALCNTITSVPPPGMEMSQLYCGGCRTLLMYTRGATSVRCSCCHTVNLVPPASNQVAHVHCGNCRTTLMYPYGAPSVKCALCHFITNTNNGRLPIPVHRPNGTNNAGTLPSTSTSMPQSQSQTVVVENPMSVDSSGKLVSNVVVGVTTDKK

SEQ ID NO: 24 - DNA, 200 bp, Glycine max
tctttgtcactttattactaatacaaacattggaaggcttccaatccctgtccatagacccaatgggacaaacaatgctggaacattaccttctacatcaacatcaatgccccaatctcaaagtcaaacggtagtggtagaaaatccaatgtctgttgattcaagtgggaaattggtgagcaatgttgttgttggcgtta

SEQ ID NO: 25 - DNA, 1150 bp, Glycine max
ggtcgctgaagaaatttccgaaagtgtgtgtgggatgagaagatgatgaggaaacattggtagaagaaattgaagagagagagagagagagagagagagagagagagggttggattggttttcttcttccttttctctaaagagaatctaatctcaatcctcattacagatttcttcattcctctctcagtcttttctgttttccccctttgacatcctcgttttcgccgaaacaaaaacaactaagattttttgtttgttttgtttttcttctccttgttgttatttgactcggttgtctgaataggttgttgcagcagggtcgggagccacagctatgcagagccaagttgtgtgcaatggttgtaggagccttctgctttacccaagaggggcaaccaatgtttgttgtgcattgtgcaacacaattacctctgttcctccccctgggatggaaatgtctcaactttattgtggagggtgtaggacattgctaatgtacacacgtggagctacaagtgtgagatgttcctgctgtcacactgtaaaccttgttccaccagcatctaatcaagtagctcatgtccattgtgggaactgccggacaacactcatgtatccttatggagctccatcagtcaaatgtgctctttgtcactttattactaatgtcagtacgaacaatggaaggcttccaatccctgtccatagacccaatgggacaaccaatgctggaacattacctactacttcaacatcaatgccccaatctcaaagtcaaacagtagtggtagaaaatccaatgtctgttgattcaagtgggaaattggtgagcaatgttgttgttggggttacaacagataagaaataacgccgtcacatataaaaggtacagttcacgtcatgacgcattaccacagttgctgtcacaggaagatattgtttgtgtatatgaatatatatgatattgcagcctgcaattgttgaattgaaattgatatttttccaatataagagctggacgaatatcatgtacgtatgtatgaatgtatatgaggtggacaagtcgtgaaaagggcttaaaacctgttttgtgtgattaccaaagcctactttgttgtttccttggtagccatacccaaatcaatta

SEQ ID NO: 26 - protein, 177 aa, Glycine max
MQSQVVCNGCRSLLLYPRGATNVCCALCNTITSVPPPGMEMSQLYCGGCRTLLMYTRGATSVRCSCCHTVNLVPPASNQVAHVHCGNCRTTLMYPYGAPSVKCALCHFITNVSTNNGRLPIPVHRPNGTTNAGTLPTTSTSMPQSQSQTVVVENPMSVDSSGKLVSNVVVGVTTDKK

SEQ ID NO: 27 - DNA, 939 bp, Glycine max
atgaccatcctcattgagcaacctgcccttgagttacaagttgaaggcaacaatgtgcatgctgaagaaaccaatgagcttgtattggatggtggatttccattgccaaaggatggatatatggccccagaaatcaattcatttggccactccttcagagaatatgatgctgaaagtgagaggcaaaaaggtgtggaggaattttataggttgcaacacatcaaccagacatatgactttgtgaagagaatgcgggaggaatatgggaaattggacaaagctgaaatgggcatttgggaatgttgtgagctgctgaatgaattggtagatgagagcgatcctgatttggacgaacctcaaattcaacatttgttacagtccgctgagaccatcagaaaagactatcctaatgaagattggctgcatttgaccgcactcatccatgatcttggaaagattcttgcgcttcctagctttggtgagcttcctcagtgggctgttgttggagatacatttcctctgggctgtgcctttgatgagtcaaatgttcatcataagtatttcaaggacaacccggattacaaatgccctgcttatagcactaaaaatgggatctacacagaagggtgtggattagacaacatagtgatgtcatggggacatgatgattacatgtatatggttgccaaggcaaatggcaccactttgccatctgcaggattgttcattatcagatatcattctttctatccattacacaaggaaggtgcatatactcacttcatgaatgaagaagacgttgagaatttgaagtggctcaaaatttttaacaaatatgatctctacagcaagagcaaagttctagttgatgtggagaaagttaagccatactatgtgtcactcattgagaagtatttccctgccaaggttagatggtga

SEQ ID NO: 28 - protein, 312 aa, Glycine max
MTILIEQPALELQVEGNNVHAEETNELVLDGGFPLPKDGYMAPEINSFGHSFREYDAESERQKGVEEFYRLQHINQTYDFVKRMREEYGKLDKAEMGIWECCELLNELVDESDPDLDEPQIQHLLQSAETIRKDYPNEDWLHLTALIHDLGKILALPSFGELPQWAVVGDTFPLGCAFDESNVHHKYFKDNPDYKCPAYSTKNGIYTEGCGLDNIVMSWGHDDYMYMVAKANGTTLPSAGLFIIRYHSFYPLHKEGAYTHFMNEEDVENLKWLKIFNKYDLYSKSKVLVDVEKVKPYYVSLIEKYFPAKVRW

SEQ ID NO: 29 - DNA, 214 bp, Glycine max
attcaacatttgttacagtctgctgagaccatcagaaaagactatcctaatgaagattggctgcatttgaccgcactcatccatgatcttggaaagattcttgcgcttcctagctttggtgagcttcctcagtgggctgttgtaggagatacatttcctctgggctgtgcctttgatgagtcaaatgttcatcataagtatttcaaggacaacc

SEQ ID NO: 30 - DNA, 551 bp, Glycine max
ttagaaatgggtatatgggagtgttgtgagctgctcaatgaagtgacggatgatagcgatcctgatttggatgaaccacaaatacaacatttgttgcagtccgctgaagccattacaaaagactatcctaatgaagattggttacatttaactgctcttattcatgatcttggaaagatccttatgcttccaagctttggtggccttcctcaatggtctgttgttggagatacatttcccctcggatgtgcttttgatgagtcaaatgttcaccacaagcatttcaaggacaatccggataacacaaatcctacttataacacgaaaaatggaatctacaaagaaggaattggactagacaatgttgtgatgtcatggggacatgatgagtatatgtatttggttgcaaaggaaaacggcaccactttgcctccagtagcattgttcattatcaaataccattctttttacgctttacatagggcaggagcatatacacatttgatgaatgaagaagatattgagaatttgaagtggctcaaaatatttag

SEQ ID NO: 31 - protein, 183 aa, Glycine max
LEMGIWECCELLNEVTDDSDPDLDEPQIQHLLQSAEAITKDYPNEDWLHLTALIHDLGKILMLPSFGGLPQWSVVGDTFPLGCAFDESNVHHKHFKDNPDNTNPTYNTKNGIYKEGIGLDNVVMSWGHDEYMYLVAKENGTTLPPVALFIIKYHSFYALHRAGAYTHLMNEEDIENLKWLKIF

SEQ ID NO: 32 - DNA, 1227 bp, Gossypium hirsutum
tcactatacattgttttttcgtttagatatttggggttcgcttctcttttcattcaagatgactatcctcattgatcaacctgattttggaattgaagcagggtttaacaaggccgatgatgttgagaaagaaggggtgttgcatgggggatttatgatgccacataccaactcttttggccacacctttagagattatcatgttgaaagtgagaggcaacagggtgttgagaccttctatcgaaccaatcatatcaaccagacatatgactttgtcaagagaatgagagaagagtacggaaatttagacagggtggagatgagcatatgggaatgctgtgagcttcttaatgatgtggttgatgagagtgaccctgacttggatgagcctcagactgaacacttgctgcaaacagctgaggctatccgaaaggactatcctgatgaggactggctgcacctcacaggccttatccatgaccttggaaaagtgcttcttcatcctagctttggagggcttcctcagtgggctgttgtaggtgatacatatcctgttggctgtgcttttgacaaatcaattgttcaccacaagtattttgaggaaaatccagactaccacaaccctgcttacaacactaaatatggagtgtactcagagggctgtggacttaacaatgttatgatgtcatgggggcatgatgactacatgtatctggtggctaaagagaacaaaacaactctgccatcagcagctcttttcattatcagataccattcattctatgccttgcataggtcaggggcatacaagcaactgatgaacggggaggatgtcgagaatctcaagtggctcgaaatattcaacaaatatgatctttacagtaagagcaaagttcggatcgatgtcgaaaaggtgaagccatactatctctccctcatagaaaagtacttcccagcaaaactaagatggtgaatccttgttctttctcactcgccacttcaattcctgggcagctgggctttaagacggtcaactagctagcgtgcttgtatatagagaataatttgatgactgagatgttatcatagttgctttgccaacagcctgtataaaaaataatggctgcaatgagtttatctatttgccttgtcatgaaagtaattctagcgtttcaacaattgtcccccaatattatttgatatctatcgtatcaaacttatttctgt

SEQ ID NO: 33 - protein, 323 aa, Gossypium hirsutum
HYTLFFRLDIWGSLLFSFKMTILIDQPDFGIEAGFNKADDVEKEGVLHGGFMMPHTNSFGHTFRDYHVESERQQGVETFYRTNHINQTYDFVKRMREEYGNLDRVEMSIWECCELLNDVVDESDPDLDEPQTEHLLQTAEAIRKDYPDEDWLHLTGLIHDLGKVLLHPSFGGLPQWAVVGDTYPVGCAFDKSIVHHKYFEENPDYHNPAYNTKYGVYSEGCGLNNVMMSWGHDDYMYLVAKENKTTLPSAALFIIRYHSFYALHRSGAYKQLMNGEDVENLKWLEIFNKYDLYSKSKVRIDVEKVKPYYLSLIEKYFPAKLRW

SEQ ID NO: 34 - DNA, 1148 bp, Gossypium hirsutum
  misc_feature (10)..(10): n is a, c, g, or t
cacttttctngcaagactctcatccatctccacgcgtccgtttttgggttttcagacagtattttcttgggaggaaaatgactatccttatcgagaagcctgagctagactgccagattcatgtggatgaaagtaaggaattggtgttggatggtggattcccagtgccgaaatctttgtcaggagaaggatttttggcaccagaggtcaattcatttggcaactcctttagggattacaatgcagaaagtgaaaggcaaaagagcgtggaggaattctacaagcagcaacatgttaaccagacatacgactttgtgcaaaagatgagggaagaatattcgaagctgaatagaatggaaatgagcatatgggaatgctgtgaattgctgaatgaggtggtggatgacagtgaccctgacctggatgaacctcaaattcagcacctcttgcagtcggctgaagctattagaaaagattatcctaatgaagattggctgcatttgactgccctcattcatgatcttgggaaggttcttcttctacctaaatttggagggcttccacaatgggctgttgttggcgacacatttcctcttgggtgtgcttttgatgaggccaatattcatcacaggtatttcaaggaaaacccagattacaacaatccctcttataacactaagaatggaatttactgggatggctgtggccttgacaatgttacaatttcatggggacatgatgattacatgtatttggtagccaaggaaaatggaaccactctaccttcagcagggctgttcattatccgatatcattcactttatcctttacataaggaggaagcgtacatgcagtttcttaatgatgaggataaggagaatctgaagtggcttagaatattcaacaagtatgacctgtacagcaagagcaaggtcgctgtggacgttgaaaaagtgaagccatattatctttcgcttattgaaaaatattttccggcaaagctcaagtggtgataggttaaagaaatgaaagaaataaacggcggcttggtgctcttcactttctatgtttgatctaattatgtaaaaaataaattgaaaattctttcctccttttgtatacatttaaatataaatattgttccatcg

SEQ ID NO: 35 - protein, 336 aa, Gossypium hirsutum
  misc_feature (3)..(3): Xaa can be any naturally occurring amino acid
LFXQDSHPSPRVRFWVFRQYFLGRKMTILIEKPELDCQIHVDESKELVLDGGFPVPKSLSGEGFLAPEVNSFGNSFRDYNAESERQKSVEEFYKQQHVNQTYDFVQKMREEYSKLNRMEMSIWECCELLNEVVDDSDPDLDEPQIQHLLQSAEAIRKDYPNEDWLHLTALIHDLGKVLLLPKFGGLPQWAVVGDTFPLGCAFDEANIHHRYFKENPDYNNPSYNTKNGIYWDGCGLDNVTISWGHDDYMYLVAKENGTTLPSAGLFIIRYHSLYPLHKEEAYMQFLNDEDKENLKWLRIFNKYDLYSKSKVAVDVEKVKPYYLSLIEKYFPAKLKW

SEQ ID NO: 36 - DNA, 719 bp, Beta vulgaris
cccacgcgtccgactttttagtgtattattaatccttcttatttacatctattaaatttgtttttctttcttttctcaagattaattatatctttcattaattattcttcgttgttgctgcttcatcatcatcatctataagaatatgactgttatcgttgaagaacctgtttttgaaacacaagaggaaaccaagaaaatttgcttggataccaatgaattggtactagatgctggatttaaaatgcctgaaccaaaagatttggtgtcaaacaatggattttcgacacccgaaaacaatgcatttggcaatacattcagagattatgatgcagaaagtgaaagacaaaaatctgttgaggaattctacaagcagaatcacatccaccaaacagttgactttgtgaaaagaatgagggaggaatacaagaaattggacaaggtgaaaatgagcatatgggaatgctgtgaacttttaaacacagttgtggatgagagtgatcctgacttagatgagcctcaaattgagcatttgctacaaactgctgaggcaattaggaaggattaccctaatgaagattggctacatttaactgcacttattcatgatcttggaaaagttcttgttcatccccagcttcggagaggctcctcaatgggcaggtgtcggcgacacgttccccgttggatgtgcatttgatgaatctattgttcatcat

SEQ ID NO: 37 - protein, 184 aa, Beta vulgaris
MTVIVEEPVFETQEETKKICLDTNELVLDAGFKMPEPKDLVSNNGFSTPENNAFGNTFRDYDAESERQKSVEEFYKQNHIHQTVDFVKRMREEYKKLDKVKMSIWECCELLNTVVDESDPDLDEPQIEHLLQTAEAIRKDYPNEDWLHLTALIHDLGKVLVHPQLRRGSSMGRCRRHVPRWMCI

SEQ ID NO: 38 - DNA, 915 bp, Zea mays
atggcgatgaatggtcgtcatggcgcagatgcggtggcggagaggaaagtccccggcggaggtgaccccgcggagctggtgctcgacgccggcttcgtcgtgccggacgccaacgccttcggcaataccttcagggactacgacgcggagtcggagcggaagcagacggtagaggagttctaccgggtgaaccacgtgaggcagacgcacgagttcgtggcgcggatgcgggcggagtacgggcggctggacaagacggagatgggcatctgggagtgcatcgagctgctgaacgagttcatcgacgacagcgacccggacctggacatgccccagatcgagcacctgctgcagaccgccgaggccatccgcaaggactaccccgacgaggactggctccacctcaccggactcatccacgacctgggcaaggtgctgctgcacccaagcttcggggagctccctcagtgggctgtcgtcggtgacaccttccccgtcggctgcgcatacgacgagtgcaacgtccacttcaagtacttcaaggagaaccccgactaccacaacccgaagctcaacaccaagttgggggtctactcggagggctgcggcctcaacaaggtgctcatgtcatggggccacgacgactacatgtacctggtggccaaggagaacaagtgcacccttccttccgcggggctgttcatcatcagataccactcgttctaccccctgcacaagcatggagcctacacacacctgatggacgatgaggacaaggagaacctcaagtggctgcatgtgttcaacaagtatgacctgtacagcaagagcaacagcaggatcgacgtggaggaggtgaagccctactacatgtccctaatcgacaagtacttcccggccaagctaagatggtga

SEQ ID NO: 39 - protein, 304 aa, Zea mays
MAMNGRHGADAVAERKVPGGGDPAELVLDAGFVVPDANAFGNTFRDYDAESERKQTVEEFYRVNHVRQTHEFVARMRAEYGRLDKTEMGIWECIELLNEFIDDSDPDLDMPQIEHLLQTAEAIRKDYPDEDWLHLTGLIHDLGKVLLHPSFGELPQWAVVGDTFPVGCAYDECNVHFKYFKENPDYHNPKLNTKLGVYSEGCGLNKVLMSWGHDDYMYLVAKENKCTLPSAGLFIIRYHSFYPLHKHGAYTHLMDDEDKENLKWLHVFNKYDLYSKSNSRIDVEEVKPYYMSLIDKYFPAKLRW

SEQ ID NO: 40 - DNA, 423 bp, Solanum tuberosum
caagagaaggagagaaagagaatgggacaacgttgccatcagctggtcttttcatcgttagatatcattcattttatgccctgcataagtctggagcttacaaggaactaatgaatgaggaagataaggaaaatcttaagtggcttcatatttttaacaaatatgacttgtacagcaaaagcaaagttcaggttaatgtggaagaggtcaagccttactacatgtctctaattgaaaagtatttcccagcaaagctgaaatggtgaagagctgacaaatgaaaatataaataataaagactgtaatggtctatagttgaatagtttttaattatttttttttatctttctttaatttatgtcttttgtattaatgttttgcgatgtaaaaatatgatccggctatgtaataaaagtggctgtt

SEQ ID NO: 41 - protein, 87 aa, Solanum tuberosum
REGEKENGTTLPSAGLFIVRYHSFYALHKSGAYKELMNEEDKENLKWLHIFNKYDLYSKSKVQVNVEEVKPYYMSLIEKYFPAKLKW

SEQ ID NO: 42 - DNA, 609 bp, Glycine max
gaagccacgtcatgaagagtatatcatttcagtaatgttttgagacgcctctataatgctttaccaacaaaacaaaacaaaaaaaagaacatttgaaaccatttgtattaaaaaaaaaaaggtatattaggccataatattataggtaacatgaaatatcaaatgacacgcaagagttttgtcaaaaatgaaaccatcacacatcagagattatggcaaataatgttttgtgtgtctcttgcttcacccataacataagcctctataactggagagaagaaaaaaaaaagtggaggggctagggtgggaatttggaagaatacagttatattgagcattgagcaagttgatagaaagcttctcaatttgtacaaaatttgcatccacatgattattaaagacgtagacagcacttcttccttctttttttctataagtttcttatatattgttcttcatgttttaatattattactttatgtacgcgtctaacagtagtcctcccaaactgctataaatagagcctcttcaacgcacctcttggcagtacaaaaattattcatctcttctaagttctaattttctaagcattcagtaaaagaactaacc

SEQ ID NO: 43 - DNA, 1999 bp, Arabidopsis thaliana
gtagtgcccttcatggataccaaaagagaaaatttgatttagtgcatacatataacaatataacgccgcataataatactgtataaaacagtcatgtaacgatatgacagcagtaatacagttccaagagacgttataatcgtatgcaatcatatgcttgcgtagattttccaacagttttgtttcgttgataggaggaactcaacactctagggtagtgattggtagacactattagcacaaaaaatattaattttactctgatgtttaccaaaaaagttaccaatcaaatatttaagagatcgtactcttccacggcgactctaaaaaccaaagatataggttagactcataactactttataaagaaaatgtttaacgataactaccgagatctaataaataaaccttcattttcaagtatattatatttgcttcttttgtttatatatcaaaccaagttctggtttataaaaatattagataaaactcgtctaaataggtaggtgtaaaataaaattttaaatttttatcgataatatttaaaatttgaaaagttaataatgatccacacattttttctaatatttaatttagtaatttttgtattaaataaaatttcaatcatatacattcgatttttctatacattttaactatctatttctgcataataaactgtattttcattttatacgcttcatcttatggatgatatttaaattttaaatagtaattcatacactttttaatatttaatttagtattttcttaaatccaaattttaatcttacaatttaaatatctactttaacataatacaaatacaatttaatttcattgtattaaattcaaatataatttgattataataaaatacaatttaattctaaaaagtccatcttagattttaattttcctttttagttttgaaaattaaaaatttaaatttattagatatatatgttactttttcagttttcctatttatttaagaaaaaaatattttttaacacatgtcaacttgtaaacaatagactgaacacgtcattttatattatgtttagttttgaaaattaaagttaattaaatatttatatttcttttttttagcttttctaattatttttaaaatagtaaatatttttaatacaaatcaatatctgaacaatagatttgatacataacataatcctataaattattaacttggaaaacgatagtttatataataaaattattttcttaagttctctaaccataacaattaaactatattttagcgaagaaaagaagagaataccgagagaacgcaacttgcactaaaagctaccactttggcaaatcactcatttatattattatatactatcacctcaattcaatcgaaacctcaaaataacactaatatatacacaaagaaacaacagaataacaccgaagaatataggtttaggaaaatccagaatttgttgagactaaagagatcaaattttcgatacaaggttttgctcaatttgtattttcataataaaattctttatttcaccatagacttacatgattagtttttcttttaataaaaaaaaacacgcgacatgaaaattatattatctcagtgttgtcgaatttgaatttgaattttgagttaaatactacacatttgttgacaacttattaaactttacaagtctgctacaaatattgtcaaatatttactaattaatggaccaaaatcctctaacttgcaaatttgtatctacatcaacttaaaaattaggaatatgcgacccaaaaaaaaaaaaactaggaataataataaaaaaatggaatgatgtggaggaagctctttactctttgagaggaagtttataaattgaccacacatttagtctattatcatcacatgtattaagacttgacaacttgtctttctcacaccaaacccctctcctctgtttcataacatctgctctttcttttttttcctaagccccta